(19) United States
(12) Patent Application Publication
(10) Pub. No.: US 2025/0051443 A1
(43) Pub. Date: Feb. 13, 2025

(54) ANTI-CD3 MONOCLONAL ANTIBODIES AND THERAPEUTIC CONSTRUCTS

(71) Applicants: NATIONAL RESEARCH COUNCIL OF CANADA, Ottawa, Ontario (CA); UNIVERSITY OF OTTAWA, Ottawa, Ontario (CA)

(72) Inventors: Anne MARCIL, Pierrefonds, Québec (CA); Robert PON, Gatineau, Québec (CA); Scott MCCOMB, Ottawa, Ontario (CA); Umar IQBAL, Stittsville, Ontario (CA)

(21) Appl. No.: 18/699,059

(22) PCT Filed: Oct. 5, 2022
(86) PCT No.: PCT/CA2022/051474

§ 371 Date: Apr. 5, 2024

Related U.S. Application Data
(60) Provisional application No. 63/321,931, filed on Mar. 21, 2022.
(60) Provisional application No. 63/253,386, filed on Oct. 7, 2021.

(51) Int. Cl. C07K 16/28

(52) U.S. Cl. CPC C07K 16/2809; C07K 16/2863; C07K 2317/24; C07K 2317/31; C07K 2317/622; C07K 2317/92

(57) ABSTRACT

Here is disclosed isolated or purified monoclonal antibodies, or an antigen-binding fragments thereof, which binds to human CD3, and which comprises: a CDRH1 amino acid sequence of SEQ ID NO: 150, a CDRH2 amino acid sequence of SEQ ID NO: 151, a CDRH3 amino acid sequence of SEQ ID NO: 152, a CDRL1 amino acid sequence of SEQ ID NO: 153, a CDRL2 amino acid sequence of SEQ ID NO: 154, and a CDRL3 amino acid sequence of SEQ ID NO: 155. Also provided are recombinant polypeptides comprising said monoclonal antibody, or an antigen-binding fragment thereof, such as multivalent antibodies, including bispecific T-cell engagers. Also described are chimeric antigen receptors (CARs) for CAR-T therapy comprising any one of the monoclonal antibodies, or antigen-binding fragments thereof. Nucleic acids encoding the aforementioned antibodies, fragments, and polypeptides are also disclosed, along with therapeutic applications in the treatment of and autoinflammatory disease or cancer.

### CROSS REFERENCE TO RELATED APPLICATIONS

This application claims the benefit of priority of U.S. Provisional Application No. 63/253,386 entitled “ANTI-BCMA SINGLE DOMAIN ANTIBODIES AND THERAPEUTIC CONSTRUCTS” and filed on Oct. 7, 2021, the contents of which are herein incorporated by reference; and of U.S. Provisional Application No. 63/321,931 entitled “ANTI-CD3 MONOCLONAL ANTIBODIES AND THERAPEUTIC CONSTRUCTS” and filed Mar. 21, 2022, the contents of which are also herein incorporated by reference.

### FIELD

The present disclosure relates generally to anti-CD3 antibodies. More particularly, the present disclosure relates to anti-CD3 monoclonal antibodies and antigen-binding fragments thereof.

### BACKGROUND

Anti-CD3 antibodies have been used in a variety of therapeutic modalities, e.g., as an immunosuppressive reagent to treat allograft rejection or chronic inflammatory and autoimmune diseases or as part of multi-specific antibodies for cancer therapy. In earlier stage applications CD3-targeting agents have also been used to specifically deliver various bioactive cargo into T cells such as mRNA- or DNA-loaded nanoparticles (Smith et al. 2017).

It is, therefore, desirable to provide immunogenic molecules with specific affinity for CD3.

### SUMMARY

It is an object of the present disclosure to obviate or mitigate at least one disadvantage of previous approaches.

In a first aspect, the present disclosure provides an isolated or purified monoclonal antibody, or an antigen-binding fragment thereof, which binds to human CD3, and which comprises:
a CDRH1 amino acid sequence of SEQ ID NO: 150,
a CDRH2 amino acid sequence of SEQ ID NO: 151,
a CDRH3 amino acid sequence of SEQ ID NO: 152,
a CDRL1 amino acid sequence of SEQ ID NO: 153,
a CDRL2 amino acid sequence of SEQ ID NO: 154, and
a CDRL3 amino acid sequence of SEQ ID NO: 155.

In one aspect, there is provided an isolated or purified monoclonal antibody, or an antigen-binding fragment thereof, which binds to human CD3, and which comprises:
a CDRH1 amino acid sequence selected from the group consisting of SEQ ID NOs: 48, 54, 60, 66, 72, 78, 84, 90, 96, 102, 108, 114, 120, 126, 132, 138, and 144,
a CDRH2 amino acid sequence selected from the group consisting of SEQ ID NOs: 49, 55, 61, 67, 73, 79, 85, 91, 97, 103, 109, 115, 121, 127, 133, 139, and 145,
a CDRH3 amino acid sequence selected from the group consisting of SEQ ID NOs: 50, 56, 62, 68, 74, 80, 86, 92, 98, 104, 110, 116, 122, 128, 134, 140, and 146,
a CDRL1 amino acid sequence selected from the group consisting of SEQ ID NOs: 51, 57, 63, 69, 75, 81, 87, 93, 99, 105, 111, 117, 123, 129, 135, 141, and 147,
a CDRL2 amino acid sequence selected from the group consisting of SEQ ID NOs: 52, 58, 64, 70, 76, 82, 88, 94, 100, 106, 112, 118, 124, 130, 136, 142, and 148, and
a CDRL3 amino acid sequence selected from the group consisting of SEQ ID NOs: 53, 59, 65, 71, 77, 83, 89, 95, 101, 106, 113, 119, 125, 131, 137, 143, and 149.

In one aspect, there is provided a monoclonal antibody, or an antigen binding fragment thereof, that competes for specific binding to human CD3 with a monoclonal antibody, or an antigen-binding fragment thereof, as described above.

In one aspect, there is provided a recombinant polypeptide comprising a monoclonal antibody, or antigen-binding fragment thereof, as defined herein.

In one aspect, there is provided a composition comprising a monoclonal antibody, or antigen-binding fragment thereof, as defined herein, or a polypeptide comprising such a monoclonal antibody, or antigen-binding fragment thereof; together with an acceptable excipient, diluent or carrier.

In one aspect, there is provided a use of the monoclonal antibody, or antigen-binding fragment thereof, as defined herein for detection of CD3.

In one aspect, there is provided the monoclonal antibody, or antigen-binding fragment thereof, as defined herein for use in detection of CD3.

In one aspect, there is provided a method of detecting CD3 in sample comprising contacting the sample with the monoclonal antibody, or antigen-binding fragment thereof, as defined herein.

In one aspect, there is provided a use of the monoclonal antibody, or antigen-binding fragment thereof, as defined herein for activation of T cells

In one aspect, there is provided a use of the monoclonal antibody, or antigen-binding fragment thereof, as defined herein for preparation of a medicament for activation of T cells.

In one aspect, there is provided the monoclonal antibody, or antigen-binding fragment thereof, as defined herein for use in activation of T cells.

In one aspect, there is provided a method of activating T cells in subject comprising administering to the subject the monoclonal antibody, or antigen-binding fragment thereof, as defined herein.

In one aspect, there is provided a use of the monoclonal antibody, or antigen-binding fragment thereof, as defined herein for expansion of T cells.

In one aspect, there is provided a use of the monoclonal antibody, or antigen-binding fragment thereof, as defined herein for preparation of a medicament for expansion of T cells.

In one aspect, there is provided the monoclonal antibody, or antigen-binding fragment thereof, as defined herein for use in expansion of T cells.

In one aspect, there is provided a method of expanding T cells comprising contacting the T cells with the monoclonal antibody, or antigen-binding fragment thereof, as defined herein.

In one aspect, there is provided a use of the monoclonal antibody, or antigen-binding fragment thereof, as defined herein for treatment of a T-cell mediated autoinflammatory disease.

In one aspect, there is provided a use of the monoclonal antibody, or antigen-binding fragment thereof, as defined herein for preparation of a medicament for treatment of a T-cell mediated autoinflammatory disease.

In one aspect, there is provided the monoclonal antibody, or antigen-binding fragment thereof, as defined herein for use in treatment of a T-cell mediated autoinflammatory disease.

In one aspect, there is provided a method of treating a T-cell mediated autoinflammatory disease in subject comprising administering to the subject the monoclonal antibody, or antigen-binding fragment thereof, as defined herein.

In one aspect, there is provided a use of the monoclonal antibody, or antigen-binding fragment thereof, as defined herein for treatment of a cancer.

In one aspect, there is provided a use of the monoclonal antibody, or antigen-binding fragment thereof, as defined herein for preparation of a medicament for treatment of a cancer.

In one aspect, there is provided the monoclonal antibody, or antigen-binding fragment thereof, as defined herein for use in treatment of a cancer.

In one aspect, there is provided a method of treating a cancer in subject comprising administering to the subject the monoclonal antibody, or antigen-binding fragment thereof, as defined herein.

In one aspect, there is provided a multivalent antibody comprising a monoclonal antibody, or an antigen-binding fragment thereof, as defined herein.

In aspect, there is provided a recombinant nucleic acid molecule encoding the multivalent antibody as defined herein.

In one aspect, there is provided a composition comprising a multivalent antibody as defined herein; together with an acceptable excipient, diluent or carrier.

In one aspect, there is provided a use of the multivalent antibody as defined herein for killing a cell.

In one aspect, there is provided a use of the multivalent antibody as defined herein for preparation of a medicament for killing a cell.

In one aspect, there is provided the multivalent antibody as defined herein for use in killing a cell.

In one aspect, there is provided a method of killing a cell comprising contacting the cell with the multivalent antibody as defined herein.

In one aspect, there is provided a use of the multivalent antibody as defined herein for killing a tumour cell.

In one aspect, there is provided a use of the multivalent antibody as defined herein for preparation of a medicament for killing a tumour cell.

In one aspect, there is provided the multivalent antibody as defined herein for use in killing a tumour cell.

In one aspect, there is provided a method of killing a tumour cell comprising contacting the tumour cell with the multivalent antibody as defined herein.

In one aspect, there is provided a use of the multivalent antibody as defined herein for treatment of a cancer.

In one aspect, there is provided a use of the multivalent antibody as defined herein for preparation of a medicament for treatment of a cancer.

In one aspect, there is provided the multivalent antibody as defined herein for use in treatment of a cancer.

In one aspect, there is provided a method of treating a cancer in subject comprising administering to the subject the multivalent antibody as defined herein.

In one aspect, there is provided a chimeric antibody receptor (CAR), which binds to human CD3, comprising the isolated or purified monoclonal antibody, or the antigen-binding fragment thereof, as defined herein.

In one aspect, there is provided a recombinant nucleic acid molecule encoding the CAR as defined herein.

In one aspect, there is provided a vector comprising the recombinant nucleic acid molecule as defined herein.

In one aspect, there is provided a recombinant viral particle comprising the recombinant nucleic acid as defined herein.

In one aspect, there is provided a cell comprising the recombinant nucleic acid molecule as defined herein.

In one aspect, there is provided an engineered cell expressing at the cell surface membrane the CAR as defined herein.

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein.

In one aspect, there is providing a method of preparing cells for CAR-T comprising introducing into a T-cell the nucleic acid or vector as described herein.

In one aspect, there is provided a use of the CAR or of the engineered cell as described herein for treatment of a cancer or an auto-immune disease.

In one aspect, there is provided a use of the CAR or of the engineered cell as described herein for preparation of a medicament treatment of a cancer or an auto-immune disease.

In one aspect, there is provided a use of the CAR or of the engineered cell as described herein for preparation of a medicament treatment of a cancer or an auto-immune disease.

In one aspect, there is provided the CAR or the engineered cell as described herein for use in treatment of a cancer or an auto-immune disease.

In one aspect there is provided a method of treating a cancer or an auto-immune disease in a subject, comprising administering to the subject the engineered cell as defined herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1A depicts a histogram obtained using flow cytometry on supernatant of selected hybridoma F282-2D5.

FIG. 1B depicts a histogram obtained using flow cytometry on supernatant of selected hybridoma F332-1E2.

FIG. 1C depicts a histogram obtained using flow cytometry on supernatant of selected hybridoma F332-5E12.

FIG. 1D depicts a histogram obtained using flow cytometry on supernatant of selected hybridoma F332-12F6.

FIG. 1E depicts a histogram obtained using flow cytometry on supernatant of an OKT-3 positive control.

FIG. 1F depicts a histogram obtained using flow cytometry on supernatant of a negative control, 3E6.

FIG. 2A depicts dose-response binding curves obtained from flow cytometry data from a first subset of purified monoclonal antibodies on a CD3 positive Jurkat T-cell line.

FIG. 2B depicts dose-response binding curves obtained from flow cytometry data from a second subset of purified monoclonal antibodies on a CD3 positive Jurkat T-cell line.

FIG. 2C depicts dose-response binding curves obtained from flow cytometry data from a third subset of purified monoclonal antibodies on a CD3 positive Jurkat T-cell line.

FIG. 2D depicts dose-response binding curves obtained from flow cytometry data from a fourth subset of purified monoclonal antibodies on a CD3 positive Jurkat T-cell line.

FIG. 3A depicts eight-point binding curves for a first subset of anti-CD3 mAbs antibodies, wherein the curves are generated from mean fluorescence intensity (MFI) values obtained through detection of labelled secondary antibody to bound CD3 in flow cytometry experiments. OTK3 served as appositive control and a non-specific mouse IgG (mIgG) as negative control.

FIG. 3B depicts eight-point binding curves for a second subset of anti-CD3 mAbs antibodies, wherein the curves are generated from mean fluorescence intensity (MFI) values obtained through detection of labelled secondary antibody to bound CD3 in flow cytometry experiments. OTK3 served as appositive control and a non-specific mouse IgG (mIgG) as negative control.

FIG. 3C depicts eight-point binding curves for a third subset of anti-CD3 mAbs antibodies, wherein the curves are generated from mean fluorescence intensity (MFI) values obtained through detection of labelled secondary antibody to bound CD3 in flow cytometry experiments. OTK3 served as appositive control and a non-specific mouse IgG (mIgG) as negative control.

FIG. 3D depicts eight-point binding curves for a fourth subset of anti-CD3 mAbs antibodies, wherein the curves are generated from mean fluorescence intensity (MFI) values obtained through detection of labelled secondary antibody to bound CD3 in flow cytometry experiments. OTK3 served as appositive control and a non-specific mouse IgG (mIgG) as negative control.

FIG. 3E depicts eight-point binding curves for a fourth subset of anti-CD3 mAbs antibodies, wherein the curves are generated from mean fluorescence intensity (MFI) values obtained through detection of labelled secondary antibody to bound CD3 in flow cytometry experiments. OTK3 served as appositive control and a non-specific mouse IgG (mIgG) as negative control.

FIG. 4 depicts binding curves showing anti-CD3 mAbs binding to Cynomolgus ex vivo PBMCs.

FIG. 5A depicts binding curves showing a first subset of anti-CD3 mAbs binding to an H. saimiri immortalized Cynomolgus HSC-F T cell line.

FIG. 5B depicts binding curves showing a second subset of anti-CD3 mAbs binding to an H. saimiri immortalized Cynomolgus HSC-F T cell line.

FIG. 5C depicts binding curves showing a third subset of anti-CD3 mAbs binding to an H. saimiri immortalized Cynomolgus HSC-F T cell line. The commercial non-human primate (NHP) cross-reactive SP34.2 anti-CD3 mAb (BD Biosciences), recombinant anti-human OKT-3 IgG2b (in house produced) and non-specific mIgG were employed as controls.

FIG. 6A depicts a graph representing the percentage of reference mAb OKT-3 maximal activation in the presence or absence of anti-CD3 mAbs measured by examining the level of dTomato-reporter gene under CD69 promoter.

FIG. 6B depicts an additional graph representing the percentage of reference mAb OKT-3 maximal activation in the presence or absence of anti-CD3 mAbs measured by examining the level of dTomato-reporter gene under CD69 promoter.

FIG. 6C depicts an additional graph representing the percentage of reference mAb OKT-3 maximal activation in the presence or absence of anti-CD3 mAbs measured by examining the level of dTomato-reporter gene under CD69 promoter.

FIG. 6D depicts an additional graph representing the percentage of reference mAb OKT-3 maximal activation in the presence or absence of anti-CD3 mAbs measured by examining the level of dTomato-reporter gene under CD69 promoter.

FIG. 7A depicts CDRH1, CDRH2, and CDRH3 sequences for sequences anti-CD3 monoclonal antibodies.

FIG. 7B is a continuation of FIG. 7A, which depicts CDRL1, CDRL2, and CDRL3 sequences for the monoclonal antibodies.

FIG. 8 is a schematic diagram of an example of a bi-specific molecule with single-chain variable fragments targeting CD3 and a different antigen which can be used to generate target cell specific T cell activation.

FIG. 9 depicts results of a bi-specific antibody (BsAb) Jurkat cell activation assay.

FIG. 10 depicts results of a primary T cell bi-specific antibody co-culture assay.

FIG. 11 is a schematic diagram depicting a bi-specific molecule with single-chain variable fragments targeting CD3 and a single-domain antibody element targeting a different antigen which can be used to generate target cell specific T cell activation.

FIG. 12 depicts results of experiments testing for biological activity of bi-specific antibodies in Jurkat co-culture assays with a lymphoma cell line (Ramos; CD22+/BCMA+/EGFR−/EGFRvIII−) or a glioblastoma cell line (U87vIII, BCMA−/CD22−/EGFR+/EGFRvIII+).

FIG. 13 depicts results of primary T cell bi-specific antibody co-culture assays for varying tumour associated antigen targeting BsAbs. Panel A depicts results of co-culture assays with a human lymphoma cell line (Ramos). Panel B depicts results of co-culture assays with a human glioblastoma cell line (U87vIII).

FIG. 14 is a schematic diagram depicting an anti-CD3 Fab format molecule.

FIG. 15 depicts results of cell binding assaying using Cy5 conjugated anti-CD3 Fab molecules. CD3-specific Cy5 labelled Fabs (1E2 or 4D6), or a control non-CD3 specific molecule (A20-Cy5) were placed at varying concentrations with CD3-expressing human Jurkat cells.

FIG. 16 is a schematic diagram depicting targeted delivery of lipid nanoparticles with anti-CD3 Fab targeting.

FIG. 17 depicts results of experiment showing delivery of mRNA cargo to CD3-expressing cells.

FIG. 18A depicts results of experiment showing delivery of mRNA cargo to primary human T cells. Graded amounts of CD3-LNPs (1E2 or 4D6) or a non-CD3-specific A20-LNP control were co-cultured with media alone. Co-cultures were incubated for 24 hours followed by assessment of the level of EGFP expression on CD4.

FIG. 18B depicts further results of experiment showing delivery of mRNA cargo to primary human T cells. Graded amounts of CD3-LNPs (1E2 or 4D6) or a non-CD3-specific A20-LNP control were co-cultured with media alone. Co-cultures were incubated for 24 hours followed by assessment of the level of EGFP expression on CD8.

FIG. 18C depicts further results of experiment showing delivery of mRNA cargo to primary human T cells. Graded amounts of CD3-LNPs (1E2 or 4D6) or a non-CD3-specific A20-LNP control were co-cultured with purified human T cells subjected to CD3/CD28 activation. Co-cultures were incubated for 24 hours followed by assessment of the level of EGFP expression on CD4.

FIG. 18D depicts further results of experiment showing delivery of mRNA cargo to primary human T cells. Graded amounts of CD3-LNPs (1E2 or 4D6) or a non-CD3-specific A20-LNP control were co-cultured with purified human T cells subjected to CD3/CD28 activation. Co-cultures were incubated for 24 hours followed by assessment of the level of EGFP expression on CD8.

### DETAILED DESCRIPTION

Generally, the present disclosure provides CD3-binding isolated or purified monoclonal antibodies, or an antigen-binding fragments thereof, which comprises: a CDRH1 amino acid sequence of SEQ ID NO: 150, a CDRH2 amino acid sequence of SEQ ID NO: 151, a CDRH3 amino acid sequence of SEQ ID NO: 152, a CDRL1 amino acid sequence of SEQ ID NO: 153, a CDRL2 amino acid sequence of SEQ ID NO: 154, and a CDRL3 amino acid sequence of SEQ ID NO: 155. Recombinant molecules comprising the monoclonal antibodies, or an antigen-binding fragments therefore, are also provided, along with therapeutic applications thereof.

Antibodies & Polypeptides Comprising them

In one aspect, there is provided an isolated or purified monoclonal antibody, or an antigen-binding fragment thereof, which binds to human CD3, and which comprises:
a CDRH1 amino acid sequence of SEQ ID NO: 150,
a CDRH2 amino acid sequence of SEQ ID NO: 151,
a CDRH3 amino acid sequence of SEQ ID NO: 152,
a CDRL1 amino acid sequence of SEQ ID NO: 153,
a CDRL2 amino acid sequence of SEQ ID NO: 154, and
a CDRL3 amino acid sequence of SEQ ID NO: 155.

The above sequences are consensus sequences derived from antibodies described herein.

“CDRs” or “complementarity-determining regions” are the portion of the variable chains in immunoglobulins that collectively constitute the paratope, and thereby impart binding specificity and affinity to the antibody. As used here, the term refers to CDRs mapped in monoclonal antibodies according to the standards or conventions set by IMGT™ (international ImMunoGeneTics information system).

An “antigen-binding fragment” is meant portion of an antibody having antigen-binding activity, including engineered antibodies and fragments thereof.

In one aspect, there is provided an isolated or purified monoclonal antibody, or an antigen-binding fragment thereof, which binds to human CD3, and which comprises:
a CDRH1 amino acid sequence selected from the group consisting of SEQ ID NOs: 48, 54, 60, 66, 72, 78, 84, 90, 96, 102, 108, 114, 120, 126, 132, 138, and 144,
a CDRH2 amino acid sequence selected from the group consisting of SEQ ID NOs: 49, 55, 61, 67, 73, 79, 85, 91, 97, 103, 109, 115, 121, 127, 133, 139, and 145,
a CDRH3 amino acid sequence selected from the group consisting of SEQ ID NOs: 50, 56, 62, 68, 74, 80, 86, 92, 98, 104, 110, 116, 122, 128, 134, 140, and 146,
a CDRL1 amino acid sequence selected from the group consisting of SEQ ID NOs: 51, 57, 63, 69, 75, 81, 87, 93, 99, 105, 111, 117, 123, 129, 135, 141, and 147,
a CDRL2 amino acid sequence selected from the group consisting of SEQ ID NOs: 52, 58, 64, 70, 76, 82, 88, 94, 100, 106, 112, 118, 124, 130, 136, 142, and 148, and
a CDRL3 amino acid sequence selected from the group consisting of SEQ ID NOs: 53, 59, 65, 71, 77, 83, 89, 95, 101, 106, 113, 119, 125, 131, 137, 143, and 149.

In one embodiment, there is provided an isolated or purified monoclonal antibody, or an antigen-binding fragment thereof, which binds to human CD3, and which comprises:
A)
i) a CDRH1 amino acid sequence of SEQ ID NO: 48,
a CDRH2 amino acid sequence of SEQ ID NO: 49,
a CDRH3 amino acid sequence of SEQ ID NO: 50,
a CDRL1 amino acid sequence of SEQ ID NO: 51,
a CDRL2 amino acid sequence of SEQ ID NO: 52, and
a CDRL3 amino acid sequence of SEQ ID NO: 53;
ii) a CDRH1 amino acid sequence of SEQ ID NO: 54,
a CDRH2 amino acid sequence of SEQ ID NO: 55,
a CDRH3 amino acid sequence of SEQ ID NO: 56,
a CDRL1 amino acid sequence of SEQ ID NO: 57,
a CDRL2 amino acid sequence of SEQ ID NO: 58, and
a CDRL3 amino acid sequence of SEQ ID NO: 59;
iii) a CDRH1 amino acid sequence of SEQ ID NO: 60,
a CDRH2 amino acid sequence of SEQ ID NO: 61,
a CDRH3 amino acid sequence of SEQ ID NO: 62,
a CDRL1 amino acid sequence of SEQ ID NO: 63,
a CDRL2 amino acid sequence of SEQ ID NO: 64, and
a CDRL3 amino acid sequence of SEQ ID NO: 65;
iv) a CDRH1 amino acid sequence of SEQ ID NO: 66,
a CDRH2 amino acid sequence of SEQ ID NO: 67,
a CDRH3 amino acid sequence of SEQ ID NO: 68,
a CDRL1 amino acid sequence of SEQ ID NO: 69,
a CDRL2 amino acid sequence of SEQ ID NO: 70, and
a CDRL3 amino acid sequence of SEQ ID NO: 71;
v) a CDRH1 amino acid sequence of SEQ ID NO: 72,
a CDRH2 amino acid sequence of SEQ ID NO: 73,
a CDRH3 amino acid sequence of SEQ ID NO: 74,
a CDRL1 amino acid sequence of SEQ ID NO: 75,
a CDRL2 amino acid sequence of SEQ ID NO: 76, and
a CDRL3 amino acid sequence of SEQ ID NO: 77;
vi) a CDRH1 amino acid sequence of SEQ ID NO: 78,
a CDRH2 amino acid sequence of SEQ ID NO: 79,
a CDRH3 amino acid sequence of SEQ ID NO: 80,
a CDRL1 amino acid sequence of SEQ ID NO: 81,
a CDRL2 amino acid sequence of SEQ ID NO: 82, and
a CDRL3 amino acid sequence of SEQ ID NO: 83;
vii) a CDRH1 amino acid sequence of SEQ ID NO: 84,
a CDRH2 amino acid sequence of SEQ ID NO: 85,
a CDRH3 amino acid sequence of SEQ ID NO: 86,
a CDRL1 amino acid sequence of SEQ ID NO: 87,
a CDRL2 amino acid sequence of SEQ ID NO: 88, and
a CDRL3 amino acid sequence of SEQ ID NO: 89;
viii) a CDRH1 amino acid sequence of SEQ ID NO: 90,
a CDRH2 amino acid sequence of SEQ ID NO: 91,
a CDRH3 amino acid sequence of SEQ ID NO: 92,
a CDRL1 amino acid sequence of SEQ ID NO: 93,
a CDRL2 amino acid sequence of SEQ ID NO: 94, and
a CDRL3 amino acid sequence of SEQ ID NO: 95;
ix) a CDRH1 amino acid sequence of SEQ ID NO: 96,
a CDRH2 amino acid sequence of SEQ ID NO: 97,
a CDRH3 amino acid sequence of SEQ ID NO: 98,
a CDRL1 amino acid sequence of SEQ ID NO: 99,
a CDRL2 amino acid sequence of SEQ ID NO: 100, and
a CDRL3 amino acid sequence of SEQ ID NO: 101;
x) a CDRH1 amino acid sequence of SEQ ID NO: 102,
a CDRH2 amino acid sequence of SEQ ID NO: 103,
a CDRH3 amino acid sequence of SEQ ID NO: 104,
a CDRL1 amino acid sequence of SEQ ID NO: 105,
a CDRL2 amino acid sequence of SEQ ID NO: 106, and
a CDRL3 amino acid sequence of SEQ ID NO: 107;
xi) a CDRH1 amino acid sequence of SEQ ID NO: 108,
a CDRH2 amino acid sequence of SEQ ID NO: 109,
a CDRH3 amino acid sequence of SEQ ID NO: 110,
a CDRL1 amino acid sequence of SEQ ID NO: 111,
a CDRL2 amino acid sequence of SEQ ID NO: 112, and
a CDRL3 amino acid sequence of SEQ ID NO: 113;
xii) a CDRH1 amino acid sequence of SEQ ID NO: 114,
a CDRH2 amino acid sequence of SEQ ID NO: 115,
a CDRH3 amino acid sequence of SEQ ID NO: 116,
a CDRL1 amino acid sequence of SEQ ID NO: 117,
a CDRL2 amino acid sequence of SEQ ID NO: 118, and
a CDRL3 amino acid sequence of SEQ ID NO: 119;
xiii) a CDRH1 amino acid sequence of SEQ ID NO: 120,
a CDRH2 amino acid sequence of SEQ ID NO: 121,
a CDRH3 amino acid sequence of SEQ ID NO: 122,
a CDRL1 amino acid sequence of SEQ ID NO: 123,
a CDRL2 amino acid sequence of SEQ ID NO: 124, and
a CDRL3 amino acid sequence of SEQ ID NO: 125;
xiv) a CDRH1 amino acid sequence of SEQ ID NO: 126,
a CDRH2 amino acid sequence of SEQ ID NO: 127,
a CDRH3 amino acid sequence of SEQ ID NO: 128,
a CDRL1 amino acid sequence of SEQ ID NO: 129,
a CDRL2 amino acid sequence of SEQ ID NO: 130, and
a CDRL3 amino acid sequence of SEQ ID NO: 131;
xv) a CDRH1 amino acid sequence of SEQ ID NO: 132,
a CDRH2 amino acid sequence of SEQ ID NO: 133,
a CDRH3 amino acid sequence of SEQ ID NO: 134,
a CDRL1 amino acid sequence of SEQ ID NO: 135,
a CDRL2 amino acid sequence of SEQ ID NO: 136, and
a CDRL3 amino acid sequence of SEQ ID NO: 137;
xvi) a CDRH1 amino acid sequence of SEQ ID NO: 138,
a CDRH2 amino acid sequence of SEQ ID NO: 139,
a CDRH3 amino acid sequence of SEQ ID NO: 140,
a CDRL1 amino acid sequence of SEQ ID NO: 141,
a CDRL2 amino acid sequence of SEQ ID NO: 142, and
a CDRL3 amino acid sequence of SEQ ID NO: 143; or
xvii) a CDRH1 amino acid sequence of SEQ ID NO: 144,
a CDRH2 amino acid sequence of SEQ ID NO: 145,
a CDRH3 amino acid sequence of SEQ ID NO: 146,
a CDRL1 amino acid sequence of SEQ ID NO: 147,
a CDRL2 amino acid sequence of SEQ ID NO: 148, and
a CDRL3 amino acid sequence of SEQ ID NO: 149;
OR
B)

CDRH1, CDRH2, CDRH3, CDRL1, CDRL2, and CDRL3 amino acid sequences that are at least 80% identical to the CDRH1, CDRH2, CDRH3, CDRL1, CDRL2, and CDRL3 sequences, respectively, defined in any one of A) i) to xvii).

In one embodiment, in B) the CDRH1 CDRH2, CDRH3, CDRL1, CDRL2, and CDRL3 amino acid sequences are at least 90% identical to the CDRH1 CDRH2, CDRH3, CDRL1, CDRL2, and CDRL3 sequences defined in any one of part A) i) to xvii). In one embodiment, in B) the CDRH1 CDRH2, CDRH3, CDRL1, CDRL2, and CDRL3 amino acid sequences are at least 95% identical to the CDRH1 CDRH2, CDRH3, CDRL1, CDRL2, and CDRL3 sequences defined in any one of part A) i) to xvii).

In one embodiment, the CDRH1 CDRH2, CDRH3, CDRL1, CDRL2, and CDRL3 amino acid sequences have at most three substitutions compared to the CDRH1 CDRH2, CDRH3, CDRL1, CDRL2, and CDRL3 sequences defined in any one of part A) i) to xvii). In one embodiment, the CDRH1 CDRH2, CDRH3, CDRL1, CDRL2, and CDRL3 amino acid sequences have at most two substitutions compared to the CDRH1 CDRH2, CDRH3, CDRL1, CDRL2, and CDRL3 sequences defined in any one of part A) i) to xvii). In one embodiment, the CDRH1 CDRH2, CDRH3, CDRL1, CDRL2, and CDRL3 amino acid sequences have at most one substitution compared to the CDRH1 CDRH2, CDRH3, CDRL1, CDRL2, and CDRL3 sequences defined in any one of part A) i) to xvii). In some embodiments, sequence differences vs. the sequences set forth in A) are conservative sequence substitutions.

The term “conservative amino acid substitutions” which is known in the art is defined herein as follows, with conservative substitutable candidate amino acids showing in parentheses: Ala (Gly, Ser); Arg (Gly, Gln); Asn (Gln; His); Asp (Glu); Cys (Ser); Gln (Asn, Lys); Glu (Asp); Gly (Ala, Pro); His (Asn; Gln); Ile (Leu; Val); Leu (lie; Val); Lys (Arg; Gln); Met (Leu, lie); Phe (Met, Leu, Tyr); Ser (Thr; Gly); Thr (Ser; Val); Trp (Tyr); Tyr (Trp; Phe); Val (lie; Leu).

Sequence variants according to certain embodiments are intended to encompass molecules in which binding affinity and/or specificity is substantially unaltered vs. the parent molecule from which it is derived. Such parameters can be readily tested, e.g., using techniques described herein and techniques known in the art. Such embodiments may encompass sequence substitutions, insertions, or deletions.

In some embodiments, sequence variation of is outside of the CDR sequences.

In one embodiment, the isolated or purified monoclonal antibody, or the antigen-binding fragment thereof, is as defined as in A).

In one embodiment, the isolated or purified monoclonal antibody, or the antigen-binding fragment thereof, comprises a heavy chain sequence selected from the group consisting of SEQ ID NOs: 1, 3, 5, 7, 9, 11, 13, 15, 17, 19, 21, 23, 25, 27, 29, 30, 31, and 33.

In one embodiment, the isolated or purified monoclonal antibody, or the antigen-binding fragment thereof, comprises a light chain sequence selected from the group consisting of SEQ ID NOs: 2, 4, 6, 8, 10, 12, 14, 16, 18, 20, 22, 24, 26, 28, 30, 32, and 34.

In some embodiments, the isolated or purified monoclonal antibody, or the antigen-binding fragment thereof, comprises a heavy chain sequence selected from the group consisting of SEQ ID NOs: 1, 3, 5, 7, 9, 11, 13, 15, 17, 19, 21, 23, 25, 27, 29, 30, 31, and 33; in combination with a light chain sequence selected from the group consisting of SEQ ID NOs: 2, 4, 6, 8, 10, 12, 14, 16, 18, 20, 22, 24, 26, 28, 30, 32, and 34.

In one embodiment, the isolated or purified monoclonal antibody, or the antigen-binding fragment thereof, comprises:
A)
a) a heavy chain amino acid sequence of SEQ ID NO: 1 and a light chain amino acid sequence of SEQ ID NO: 2,
b) a heavy chain amino acid sequence of SEQ ID NO: 3 and a light chain amino acid sequence of SEQ ID NO: 4,
c) a heavy chain amino acid sequence of SEQ ID NO: 5 and a light chain amino acid sequence of SEQ ID NO: 6,
d) a heavy chain amino acid sequence of SEQ ID NO: 7 and a light chain amino acid sequence of SEQ ID NO: 8,
e) a heavy chain amino acid sequence of SEQ ID NO: 9 and a light chain amino acid sequence of SEQ ID NO: 10,
f) a heavy chain amino acid sequence of SEQ ID NO: 11 and a light chain amino acid sequence of SEQ ID NO: 12,
g) a heavy chain amino acid sequence of SEQ ID NO: 13 and a light chain amino acid sequence of SEQ ID NO: 14,
h) a heavy chain amino acid sequence of SEQ ID NO: 15 and a light chain amino acid sequence of SEQ ID NO: 16,
i) a heavy chain amino acid sequence of SEQ ID NO: 17 and a light chain amino acid sequence of SEQ ID NO: 18,
j) a heavy chain amino acid sequence of SEQ ID NO: 19 and a light chain amino acid sequence of SEQ ID NO: 20,
k) a heavy chain amino acid sequence of SEQ ID NO: 21 and a light chain amino acid sequence of SEQ ID NO: 22,
l) a heavy chain amino acid sequence of SEQ ID NO: 23 and a light chain amino acid sequence of SEQ ID NO: 24,
m) a heavy chain amino acid sequence of SEQ ID NO: 25 and a light chain amino acid sequence of SEQ ID NO: 26,
n) a heavy chain amino acid sequence of SEQ ID NO: 27 and a light chain amino acid sequence of SEQ ID NO: 28,
o) a heavy chain amino acid sequence of SEQ ID NO: 29 and a light chain amino acid sequence of SEQ ID NO: 30,
p) a heavy chain amino acid sequence of SEQ ID NO: 31 and a light chain amino acid sequence of SEQ ID NO: 32, or
q) a heavy chain amino acid sequence of SEQ ID NO: 33 and a light chain amino acid sequence of SEQ ID NO: 34; or
B)
a heavy chain amino acid sequence at a light chain amino acid sequence that are at least 80% identical to the heavy chain amino acid sequences and the light chain amino acid sequences defined in defined in any one of A) a) to q).

In one embodiment, in B) the heavy chain amino acid sequence and the light chain amino acid sequence are at least 90% identical to the heavy chain amino acid sequence and the light chain amino acid sequence defined in any one of part A) a) to q). In one embodiment, in B) the heavy chain amino acid sequence and the light chain amino acid sequence are at least 95% identical to the heavy chain amino acid sequence and the light chain amino acid sequence defined in any one of part A) a) to q).

In one embodiment, in B) the heavy chain amino acid sequence and the light chain amino acid sequence have at most three substitutions compared to the heavy chain amino acid sequence and the light chain amino acid sequence defined in any one of part A) a) to q). In one embodiment, in B) the heavy chain amino acid sequence and the light chain amino acid sequence have at most two substitutions compared to t the heavy chain amino acid sequence and the light chain amino acid sequence defined in any one of part A) a) to q). In one embodiment, in B) the heavy chain amino acid sequence and the light chain amino acid sequence have at most one substitution compared to the heavy chain amino acid sequence and the light chain amino acid sequence defined in any one of part A) a) to q). In some embodiments, sequence differences vs. the sequences set forth in A) are conservative sequence substitutions.

In one embodiment, the isolated or purified monoclonal antibody, or the antigen-binding fragment thereof, is as defined as in A).

In one embodiment, the isolated or purified monoclonal antibody, or the antigen-binding fragment thereof comprises the heavy chain amino acid sequence of SEQ ID NO: 1 and the light chain amino acid sequence of SEQ ID NO: 2.

In one embodiment, the isolated or purified monoclonal antibody, or the antigen-binding fragment thereof comprises the heavy chain amino acid sequence of SEQ ID NO: 3 and the light chain amino acid sequence of SEQ ID NO: 4.

In one embodiment, the isolated or purified monoclonal antibody, or the antigen-binding fragment thereof comprises the heavy chain amino acid sequence of SEQ ID NO: 5 and the light chain amino acid sequence of SEQ ID NO: 6.

In one embodiment, the isolated or purified monoclonal antibody, or the antigen-binding fragment thereof comprises the heavy chain amino acid sequence of SEQ ID NO: 7 and the light chain amino acid sequence of SEQ ID NO: 8.

In one embodiment, the isolated or purified monoclonal antibody, or the antigen-binding fragment thereof comprises the heavy chain amino acid sequence of SEQ ID NO: 9 and the light chain amino acid sequence of SEQ ID NO: 10.

In one embodiment, the isolated or purified monoclonal antibody, or the antigen-binding fragment thereof comprises the heavy chain amino acid sequence of SEQ ID NO: 11 and the light chain amino acid sequence of SEQ ID NO: 12.

In one embodiment, the isolated or purified monoclonal antibody, or the antigen-binding fragment thereof comprises the heavy chain amino acid sequence of SEQ ID NO: 13 and the light chain amino acid sequence of SEQ ID NO: 14.

In one embodiment, the isolated or purified monoclonal antibody, or the antigen-binding fragment thereof comprises the heavy chain amino acid sequence of SEQ ID NO: 15 and the light chain amino acid sequence of SEQ ID NO: 16.

In one embodiment, the isolated or purified monoclonal antibody, or the antigen-binding fragment thereof comprises the heavy chain amino acid sequence of SEQ ID NO: 17 and the light chain amino acid sequence of SEQ ID NO: 18.

In one embodiment, the isolated or purified monoclonal antibody, or the antigen-binding fragment thereof comprises heavy chain amino acid sequence of SEQ ID NO: 19 and the light chain amino acid sequence of SEQ ID NO: 20.

In one embodiment, the isolated or purified monoclonal antibody, or the antigen-binding fragment thereof comprises the heavy chain amino acid sequence of SEQ ID NO: 21 and the light chain amino acid sequence of SEQ ID NO: 22.

In one embodiment, the isolated or purified monoclonal antibody, or the antigen-binding fragment thereof comprises the heavy chain amino acid sequence of SEQ ID NO: 23 and the light chain amino acid sequence of SEQ ID NO: 24.

In one embodiment, the isolated or purified monoclonal antibody, or the antigen-binding fragment thereof comprises the heavy chain amino acid sequence of SEQ ID NO: 25 and the light chain amino acid sequence of SEQ ID NO: 26.

In one embodiment, the isolated or purified monoclonal antibody, or the antigen-binding fragment thereof comprises the heavy chain amino acid sequence of SEQ ID NO: 27 and the light chain amino acid sequence of SEQ ID NO: 28.

In one embodiment, the isolated or purified monoclonal antibody, or the antigen-binding fragment thereof comprises the heavy chain amino acid sequence of SEQ ID NO: 29 and the light chain amino acid sequence of SEQ ID NO: 30.

In one embodiment, the isolated or purified monoclonal antibody, or the antigen-binding fragment thereof comprises the heavy chain amino acid sequence of SEQ ID NO: 31 and the light chain amino acid sequence of SEQ ID NO: 32.

In one embodiment, the isolated or purified monoclonal antibody, or the antigen-binding fragment thereof comprises the heavy chain amino acid sequence of SEQ ID NO: 33 and the light chain amino acid sequence of SEQ ID NO: 34.

In one embodiment, the isolated or purified monoclonal antibody, or the antigen-binding fragment thereof comprises CDRH1, CDRH2, and CDRH3 sequences of SEQ ID NO: 1 and CDRL1, CDRL2, and CDRL3 sequence of SEQ ID NO: 2.

In one embodiment, the isolated or purified monoclonal antibody, or the antigen-binding fragment thereof comprises CDRH1, CDRH2, and CDRH3 sequences of SEQ ID NO: 3 and CDRL1, CDRL2, and CDRL3 sequence of SEQ ID NO: 4.

In one embodiment, the isolated or purified monoclonal antibody, or the antigen-binding fragment thereof comprises CDRH1, CDRH2, and CDRH3 sequences of SEQ ID NO: 5 and CDRL1, CDRL2, and CDRL3 sequence of SEQ ID NO: 6.

In one embodiment, the isolated or purified monoclonal antibody, or the antigen-binding fragment thereof comprises CDRH1, CDRH2, and CDRH3 sequences of SEQ ID NO: 7 and CDRL1, CDRL2, and CDRL3 sequence of SEQ ID NO: 8.

In one embodiment, the isolated or purified monoclonal antibody, or the antigen-binding fragment thereof comprises CDRH1, CDRH2, and CDRH3 sequences of SEQ ID NO: 9 and CDRL1, CDRL2, and CDRL3 sequence of SEQ ID NO: 10.

In one embodiment, the isolated or purified monoclonal antibody, or the antigen-binding fragment thereof comprises CDRH1, CDRH2, and CDRH3 sequences of SEQ ID NO: 11 and CDRL1, CDRL2, and CDRL3 sequence of SEQ ID NO: 12.

In one embodiment, the isolated or purified monoclonal antibody, or the antigen-binding fragment thereof comprises CDRH1, CDRH2, and CDRH3 sequences of SEQ ID NO: 13 and CDRL1, CDRL2, and CDRL3 sequence of SEQ ID NO: 14.

In one embodiment, the isolated or purified monoclonal antibody, or the antigen-binding fragment thereof comprises CDRH1, CDRH2, and CDRH3 sequences of SEQ ID NO: 15 and CDRL1, CDRL2, and CDRL3 sequence of SEQ ID NO: 16.

In one embodiment, the isolated or purified monoclonal antibody, or the antigen-binding fragment thereof comprises CDRH1, CDRH2, and CDRH3 sequences of SEQ ID NO: 17 and CDRL1, CDRL2, and CDRL3 sequence of SEQ ID NO: 18.

In one embodiment, the isolated or purified monoclonal antibody, or the antigen-binding fragment thereof comprises CDRH1, CDRH2, and CDRH3 sequences of SEQ ID NO: 19 and CDRL1, CDRL2, and CDRL3 sequence of SEQ ID NO: 20.

In one embodiment, the isolated or purified monoclonal antibody, or the antigen-binding fragment thereof comprises CDRH1, CDRH2, and CDRH3 sequences of SEQ ID NO: 21 and CDRL1, CDRL2, and CDRL3 sequence of SEQ ID NO: 22.

In one embodiment, the isolated or purified monoclonal antibody, or the antigen-binding fragment thereof comprises CDRH1, CDRH2, and CDRH3 sequences of SEQ ID NO: 23 and CDRL1, CDRL2, and CDRL3 sequence of SEQ ID NO: 24.

In one embodiment, the isolated or purified monoclonal antibody, or the antigen-binding fragment thereof comprises CDRH1, CDRH2, and CDRH3 sequences of SEQ ID NO: 25 and CDRL1, CDRL2, and CDRL3 sequence of SEQ ID NO: 26.

In one embodiment, the isolated or purified monoclonal antibody, or the antigen-binding fragment thereof comprises CDRH1, CDRH2, and CDRH3 sequences of SEQ ID NO: 27 and CDRL1, CDRL2, and CDRL3 sequence of SEQ ID NO: 28.

In one embodiment, the isolated or purified monoclonal antibody, or the antigen-binding fragment thereof comprises CDRH1, CDRH2, and CDRH3 sequences of SEQ ID NO: 29 and CDRL1, CDRL2, and CDRL3 sequence of SEQ ID NO: 30.

In one embodiment, the isolated or purified monoclonal antibody, or the antigen-binding fragment thereof comprises CDRH1, CDRH2, and CDRH3 sequences of SEQ ID NO: 31 and CDRL1, CDRL2, and CDRL3 sequence of SEQ ID NO: 32.

In one embodiment, the isolated or purified monoclonal antibody, or the antigen-binding fragment thereof comprises CDRH1, CDRH2, and CDRH3 sequences of SEQ ID NO: 33 and CDRL1, CDRL2, and CDRL3 sequence of SEQ ID NO: 34.

In one embodiment, the isolated or purified monoclonal antibody, or the antigen-binding fragment thereof comprises:
a CDRH1 amino acid sequence of SEQ ID NO: 48,
a CDRH2 amino acid sequence of SEQ ID NO: 49,
a CDRH3 amino acid sequence of SEQ ID NO: 50,
a CDRL1 amino acid sequence of SEQ ID NO: 51,
a CDRL2 amino acid sequence of SEQ ID NO: 52, and
a CDRL3 amino acid sequence of SEQ ID NO: 53.

In one embodiment, the isolated or purified monoclonal antibody, or the antigen-binding fragment thereof comprises:
a CDRH1 amino acid sequence of SEQ ID NO: 54,
a CDRH2 amino acid sequence of SEQ ID NO: 55,
a CDRH3 amino acid sequence of SEQ ID NO: 56,
a CDRL1 amino acid sequence of SEQ ID NO: 57,
a CDRL2 amino acid sequence of SEQ ID NO: 58, and
a CDRL3 amino acid sequence of SEQ ID NO: 59.

In one embodiment, the isolated or purified monoclonal antibody, or the antigen-binding fragment thereof comprises:
a CDRH1 amino acid sequence of SEQ ID NO: 60,
a CDRH2 amino acid sequence of SEQ ID NO: 61,
a CDRH3 amino acid sequence of SEQ ID NO: 62,
a CDRL1 amino acid sequence of SEQ ID NO: 63,
a CDRL2 amino acid sequence of SEQ ID NO: 64, and
a CDRL3 amino acid sequence of SEQ ID NO: 65.

In one embodiment, the isolated or purified monoclonal antibody, or the antigen-binding fragment thereof comprises:
a CDRH1 amino acid sequence of SEQ ID NO: 66,
a CDRH2 amino acid sequence of SEQ ID NO: 67,
a CDRH3 amino acid sequence of SEQ ID NO: 68,
a CDRL1 amino acid sequence of SEQ ID NO: 69,
a CDRL2 amino acid sequence of SEQ ID NO: 70, and
a CDRL3 amino acid sequence of SEQ ID NO: 71.

In one embodiment, the isolated or purified monoclonal antibody, or the antigen-binding fragment thereof comprises:
a CDRH1 amino acid sequence of SEQ ID NO: 72,
a CDRH2 amino acid sequence of SEQ ID NO: 73,
a CDRH3 amino acid sequence of SEQ ID NO: 74,
a CDRL1 amino acid sequence of SEQ ID NO: 75,
a CDRL2 amino acid sequence of SEQ ID NO: 76, and
a CDRL3 amino acid sequence of SEQ ID NO: 77.

In one embodiment, the isolated or purified monoclonal antibody, or the antigen-binding fragment thereof comprises:
a CDRH1 amino acid sequence of SEQ ID NO: 78,
a CDRH2 amino acid sequence of SEQ ID NO: 79,
a CDRH3 amino acid sequence of SEQ ID NO: 80,
a CDRL1 amino acid sequence of SEQ ID NO: 81,
a CDRL2 amino acid sequence of SEQ ID NO: 82, and
a CDRL3 amino acid sequence of SEQ ID NO: 83.

In one embodiment, the isolated or purified monoclonal antibody, or the antigen-binding fragment thereof comprises:
a CDRH1 amino acid sequence of SEQ ID NO: 84,
a CDRH2 amino acid sequence of SEQ ID NO: 85,
a CDRH3 amino acid sequence of SEQ ID NO: 86,
a CDRL1 amino acid sequence of SEQ ID NO: 87,
a CDRL2 amino acid sequence of SEQ ID NO: 88, and
a CDRL3 amino acid sequence of SEQ ID NO: 89.

In one embodiment, the isolated or purified monoclonal antibody, or the antigen-binding fragment thereof comprises:
a CDRH1 amino acid sequence of SEQ ID NO: 90,
a CDRH2 amino acid sequence of SEQ ID NO: 91,
a CDRH3 amino acid sequence of SEQ ID NO: 92,
a CDRL1 amino acid sequence of SEQ ID NO: 93,
a CDRL2 amino acid sequence of SEQ ID NO: 94, and
a CDRL3 amino acid sequence of SEQ ID NO: 95.

In one embodiment, the isolated or purified monoclonal antibody, or the antigen-binding fragment thereof comprises:
a CDRH1 amino acid sequence of SEQ ID NO: 96,
a CDRH2 amino acid sequence of SEQ ID NO: 97,
a CDRH3 amino acid sequence of SEQ ID NO: 98,
a CDRL1 amino acid sequence of SEQ ID NO: 99,
a CDRL2 amino acid sequence of SEQ ID NO: 100, and
a CDRL3 amino acid sequence of SEQ ID NO: 101.

In one embodiment, the isolated or purified monoclonal antibody, or the antigen-binding fragment thereof comprises:
a CDRH1 amino acid sequence of SEQ ID NO: 102,
a CDRH2 amino acid sequence of SEQ ID NO: 103,
a CDRH3 amino acid sequence of SEQ ID NO: 104,
a CDRL1 amino acid sequence of SEQ ID NO: 105,
a CDRL2 amino acid sequence of SEQ ID NO: 106, and
a CDRL3 amino acid sequence of SEQ ID NO: 107.

In one embodiment, the isolated or purified monoclonal antibody, or the antigen-binding fragment thereof comprises:
a CDRH1 amino acid sequence of SEQ ID NO: 108,
a CDRH2 amino acid sequence of SEQ ID NO: 109,
a CDRH3 amino acid sequence of SEQ ID NO: 110,
a CDRL1 amino acid sequence of SEQ ID NO: 111,
a CDRL2 amino acid sequence of SEQ ID NO: 112, and
a CDRL3 amino acid sequence of SEQ ID NO: 113.

In one embodiment, the isolated or purified monoclonal antibody, or the antigen-binding fragment thereof comprises:
a CDRH1 amino acid sequence of SEQ ID NO: 114,
a CDRH2 amino acid sequence of SEQ ID NO: 115,
a CDRH3 amino acid sequence of SEQ ID NO: 116,
a CDRL1 amino acid sequence of SEQ ID NO: 117,
a CDRL2 amino acid sequence of SEQ ID NO: 118, and
a CDRL3 amino acid sequence of SEQ ID NO: 119.

In one embodiment, the isolated or purified monoclonal antibody, or the antigen-binding fragment thereof comprises:
a CDRH1 amino acid sequence of SEQ ID NO: 120,
a CDRH2 amino acid sequence of SEQ ID NO: 121,
a CDRH3 amino acid sequence of SEQ ID NO: 122,
a CDRL1 amino acid sequence of SEQ ID NO: 123,
a CDRL2 amino acid sequence of SEQ ID NO: 124, and
a CDRL3 amino acid sequence of SEQ ID NO: 125.

In one embodiment, the isolated or purified monoclonal antibody, or the antigen-binding fragment thereof comprises:
a CDRH1 amino acid sequence of SEQ ID NO: 126,
a CDRH2 amino acid sequence of SEQ ID NO: 127,
a CDRH3 amino acid sequence of SEQ ID NO: 128,
a CDRL1 amino acid sequence of SEQ ID NO: 129,
a CDRL2 amino acid sequence of SEQ ID NO: 130, and
a CDRL3 amino acid sequence of SEQ ID NO: 131.

In one embodiment, the isolated or purified monoclonal antibody, or the antigen-binding fragment thereof comprises:
a CDRH1 amino acid sequence of SEQ ID NO: 132,
a CDRH2 amino acid sequence of SEQ ID NO: 133,
a CDRH3 amino acid sequence of SEQ ID NO: 134,
a CDRL1 amino acid sequence of SEQ ID NO: 135,
a CDRL2 amino acid sequence of SEQ ID NO: 136, and
a CDRL3 amino acid sequence of SEQ ID NO: 137.

In one embodiment, the isolated or purified monoclonal antibody, or the antigen-binding fragment thereof comprises:
a CDRH1 amino acid sequence of SEQ ID NO: 138,
a CDRH2 amino acid sequence of SEQ ID NO: 139,
a CDRH3 amino acid sequence of SEQ ID NO: 140,
a CDRL1 amino acid sequence of SEQ ID NO: 141,
a CDRL2 amino acid sequence of SEQ ID NO: 142, and
a CDRL3 amino acid sequence of SEQ ID NO: 143.

In one embodiment, the isolated or purified monoclonal antibody, or the antigen-binding fragment thereof comprises:
a CDRH1 amino acid sequence of SEQ ID NO: 144,
a CDRH2 amino acid sequence of SEQ ID NO: 145,
a CDRH3 amino acid sequence of SEQ ID NO: 146,
a CDRL1 amino acid sequence of SEQ ID NO: 147,
a CDRL2 amino acid sequence of SEQ ID NO: 148, and
a CDRL3 amino acid sequence of SEQ ID NO: 149.

In one embodiment, the isolated or purified monoclonal antibody, or the antigen-binding fragment thereof, is humanized.

By the term “humanized” as used herein is meant mutated so that immunogenicity upon administration in human patients is minor or nonexistent. Humanizing a polypeptide, according to the present invention, comprises a step of replacing one or more of the Camelidae amino acids by their human counterpart as found in the human consensus sequence, without that polypeptide losing its typical character, i.e. the humanization does not significantly affect the antigen binding capacity of the resulting polypeptide. A humanized antibody can be produced using a variety of techniques known in the art, including but not limited to, CDR-grafting, veneering or resurfacing, chain shuffling, etc.

In one embodiment, the isolated or purified monoclonal antibody, or the antigen-binding fragment thereof, is a Fab, an F(ab′)₂, and Fab′, or an scFV.

In one embodiment, the isolated monoclonal antibody, or the antigen-binding fragment thereof has an affinity for human CD3 of 31 nM or less.

In one embodiment, the isolated or purified monoclonal antibody, or the antigen-binding fragment thereof has an affinity for human CD3 of 30 nM or less.

In one embodiment, the isolated or purified monoclonal antibody, or the antigen-binding fragment thereof has an affinity for human CD3 of 25 nM or less.

In one embodiment, the isolated or purified monoclonal antibody, or the antigen-binding fragment thereof has an affinity for human CD3 of 20 nM or less.

In one embodiment, the isolated or purified monoclonal antibody, or the antigen-binding fragment thereof has an affinity for human CD3 of 15 nM or less.

In one embodiment, the isolated or purified monoclonal antibody, or the antigen-binding fragment thereof has an affinity for human CD3 of 10 nM or less.

In one embodiment, the isolated or purified monoclonal antibody, or the antigen-binding fragment thereof has an affinity for human CD3 of 5 nM or less.

In one embodiment, the isolated or purified monoclonal antibody, or the antigen-binding fragment thereof has an affinity for human CD3 of 2 nM or less.

In one embodiment, the isolated or purified monoclonal antibody, or the antigen-binding fragment thereof has an affinity for human CD3 of 1 nM or less.

Binding affinity can be determined, e.g., according to assays described herein.

For example, the affinity for human CD3 may determine, in some embodiments, by flow cytometry analysis of binding to human T cells.

In one embodiment, the isolated or purified monoclonal antibody, or the antigen-binding fragment thereof additionally binds to Macaca fascicularis CD3 (see GenBank Accession No. GCA_000364345.1).

In one aspect, there is provided a monoclonal antibody, or an antigen binding fragment thereof, that competes for specific binding to human CD3 with a monoclonal antibody, or an antigen-binding fragment thereof, as described above. A monoclonal antibody, or an antigen binding fragment thereof, which competes with a monoclonal antibody, or an antigen-binding fragment thereof, as described above, may be identified by a method that comprises a binding assay which assesses whether or not a test antibody is able to cross-compete with a known antibody of the invention for a binding site on the target molecule. For example, the antibodies described hereinabove may be used as reference antibodies. Methods for carrying out competitive binding assays are well known in the art. For example they may involve contacting together a known antibody of the invention and a target molecule under conditions under which the antibody can bind to the target molecule. The antibody/target complex may then be contacted with a test antibody and the extent to which the test antibody is able to displace the antibody of the invention from antibody/target complexes may be assessed. An alternative method may involve contacting a test antibody with a target molecule under conditions that allow for antibody binding, then adding an antibody of the invention that is capable of binding that target molecule and assessing the extent to which the antibody of the invention is able to displace the test antibody from antibody/target complexes. Such antibodies may be identified by generating new monoclonal antibodies to CD3 and screening the resulting library for cross-competition. Alternatively, one of the antibodies described herein may serve as a starting point for diversification, library generation, and screening. A further alternative could involve testing individual variants of an antibody described herein.

In one embodiment, the antigen binding fragment is an scFv derived from a monoclonal antibody described herein. For example, in one embodiment, the scFv may comprise SEQ ID No: 37.

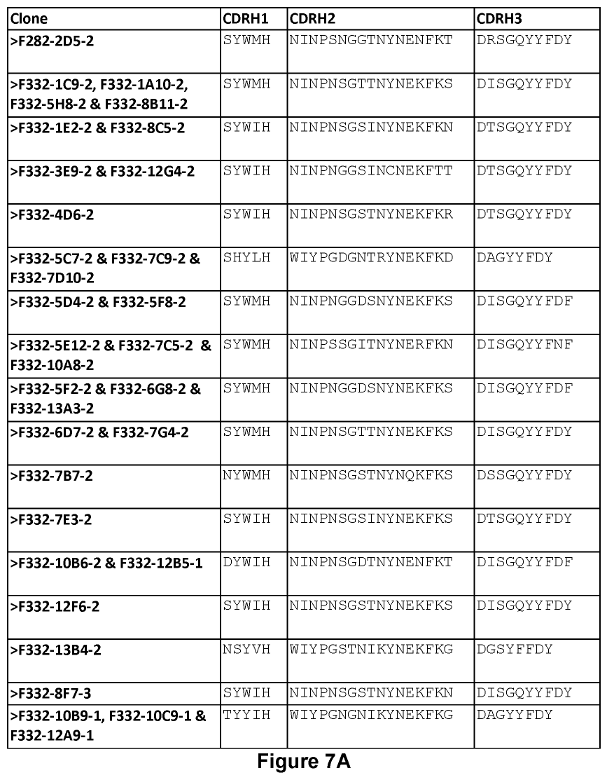

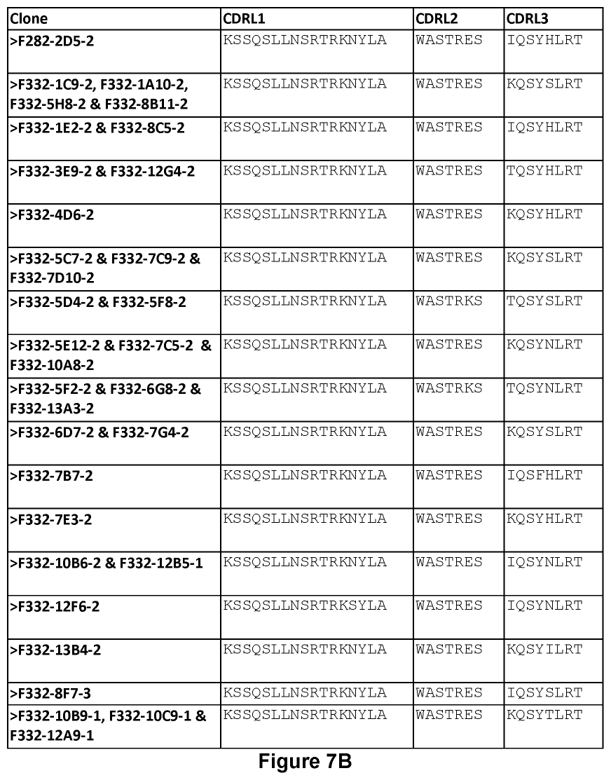

FIG. 7A, FIG. 7B, and Table 6 list the full length sequences for monoclonal antibodies disclosed herein. CDRH1, CDRH2, CDRH3, CDRL1, CDRL2, and CDRL3 sequences are underlined in Table 6. CDR identification and numbering used herein is according to the IMGT™ convention.

### Recombinant Molecule

In one aspect, there is provided a recombinant polypeptide comprising a monoclonal antibody, or antigen-binding fragment thereof, as defined herein. In one embodiment, there is provided a recombinant polypeptide comprising one or more monoclonal antibody, or antigen-binding fragment thereof, as defined herein. In one embodiment, there is provided a recombinant polypeptide comprising two or more monoclonal antibodies, or antigen-binding fragments thereof, as defined herein.

### Compositions

In one aspect, there is provided a composition comprising a monoclonal antibody, or antigen-binding fragment thereof, as defined herein, or a polypeptide comprising such a monoclonal antibody, or antigen-binding fragment thereof; together with an acceptable excipient, diluent or carrier. In one embodiment the composition is a pharmaceutical composition, and the excipient, diluent or carrier is a pharmaceutically acceptable excipient, diluent or carrier.

### Uses & Methods

In one aspect, there is provided a use of the monoclonal antibody, or antigen-binding fragment thereof, as defined herein for detection of CD3.

In one aspect, there is provided the monoclonal antibody, or antigen-binding fragment thereof, as defined herein for use in detection of CD3.

In one aspect, there is provided a method of detecting CD3 in sample comprising contacting the sample with the monoclonal antibody, or antigen-binding fragment thereof, as defined herein.

In one aspect, there is provided a use of the monoclonal antibody, or antigen-binding fragment thereof, as defined herein for activation of T cells. The monoclonal antibody, or antigen-binding fragment thereof may be for use in target antigen-specific activation of T cells.

In one aspect, there is provided a use of the monoclonal antibody, or antigen-binding fragment thereof, as defined herein for preparation of a medicament for activation of T cells. The medicament may be for use in target antigen-specific activation of T cells.

In one aspect, there is provided the monoclonal antibody, or antigen-binding fragment thereof, as defined herein for use in activation of T cells. The monoclonal antibody, or antigen-binding fragment thereof may be for use in target antigen-specific activation of T cells.

In one aspect, there is provided a method of activating T cells in subject comprising administering to the subject the monoclonal antibody, or antigen-binding fragment thereof, as defined herein.

In one aspect, there is provided a use of the monoclonal antibody, or antigen-binding fragment thereof, as defined herein for expansion of T cells.

In one aspect, there is provided a use of the monoclonal antibody, or antigen-binding fragment thereof, as defined herein for preparation of a medicament for expansion of T cells.

In one aspect, there is provided the monoclonal antibody, or antigen-binding fragment thereof, as defined herein for use in expansion of T cells.

In one aspect, there is provided a method of expanding T cells comprising contacting the T cells with the monoclonal antibody, or antigen-binding fragment thereof, as defined herein.

In one aspect, there is provided a use of the monoclonal antibody, or antigen-binding fragment thereof, as defined herein for treatment of a T-cell mediated autoinflammatory disease. In one embodiment, the T cell mediated autoinflammatory disease is multiple sclerosis, rheumatoid arthritis, ulcerative colitis, or transplant rejection.

In one aspect, there is provided a use of the monoclonal antibody, or antigen-binding fragment thereof, as defined herein for preparation of a medicament for treatment of a T-cell mediated autoinflammatory disease. In one embodiment, the T cell mediated autoinflammatory disease is multiple sclerosis, rheumatoid arthritis, ulcerative colitis, or transplant rejection.

In one aspect, there is provided the monoclonal antibody, or antigen-binding fragment thereof, as defined herein for use in treatment of a T-cell mediated autoinflammatory disease. In one embodiment, the T cell mediated autoinflammatory disease is multiple sclerosis, rheumatoid arthritis, ulcerative colitis, or transplant rejection.

In one aspect, there is provided a method of treating a T-cell mediated autoinflammatory disease in subject comprising administering to the subject the monoclonal antibody, or antigen-binding fragment thereof, as defined herein. In one embodiment, the T cell mediated autoinflammatory disease is multiple sclerosis, rheumatoid arthritis, ulcerative colitis, or transplant rejection.

In one aspect, there is provided a use of the monoclonal antibody, or antigen-binding fragment thereof, as defined herein for treatment of a cancer. In one embodiment, the cancer is a leukemia, lymphoma, multiple myeloma, lung cancer, pancreatic cancer, gastric cancer, colon cancer, or brain cancer. In one embodiment, the cancer is a leukemia.

In one aspect, there is provided a use of the monoclonal antibody, or antigen-binding fragment thereof, as defined herein for preparation of a medicament for treatment of a cancer. In one embodiment, the cancer is a leukemia, lymphoma, multiple myeloma, lung cancer, pancreatic cancer, gastric cancer, colon cancer, or brain cancer. In one embodiment, the cancer is a leukemia.

In one aspect, there is provided the monoclonal antibody, or antigen-binding fragment thereof, as defined herein for use in treatment of a cancer. In one embodiment, the cancer is a leukemia, lymphoma, multiple myeloma, lung cancer, pancreatic cancer, gastric cancer, colon cancer, or brain cancer. In one embodiment, the cancer is a leukemia.

In one aspect, there is provided a method of treating a cancer in subject comprising administering to the subject the monoclonal antibody, or antigen-binding fragment thereof, as defined herein. In one embodiment, the cancer is a leukemia, lymphoma, multiple myeloma, lung cancer, pancreatic cancer, gastric cancer, colon cancer, or brain cancer. In one embodiment, the cancer is a leukemia.

### Multivalent Antibodies & Related Embodiments

In one aspect, there is provided a multivalent antibody comprising a monoclonal antibody, or an antigen-binding fragment thereof, as defined herein.

By “multivalent antibody” is use herein to mean a molecule comprising more than one variable region or paratope for binding to one or more antigen(s) within the same or different target molecule(s).

In one embodiment, there is provided a multivalent antibody comprising:
a first antigen-binding portion comprising the monoclonal antibody, or the antigen-binding fragment thereof, as defined herein, and
a second antigen-binding portion.

The terms “first” and “second” are not intended to be reflective of N- to C-terminal order. In some embodiments, the second antigen-binding portion is positional N-terminally with respect to the first antigen-binding portion. In some embodiments, the first antigen-binding portion is positional N-terminally with respect to the second antigen-binding portion. This positioning is relative, and it will appreciated that intervening sequences, such as linkers, spacers, or other sequences, may be present in some embodiments.

In some embodiments, paratopes of said first and second antigen-binding portions may bind to different epitopes on the same target molecule. In some embodiments, the paratopes may bind to different target molecules. In these embodiments, the multivalent antibody may be termed bispecific, trispecific, or multispecific, depending on the number of paratopes of different specificity that are present. As the multivalent antibody comprises one of the anti-CD3 monoclonal antibodies, or antigen-binding fragments thereof, as herein defined, the multivalent antibody comprises CD3 binding affinity.

In one embodiment, the multivalent antibody is a bispecific antibody.

In some embodiments, the second antigen binding portion may comprise a monoclonal antibody, an Fab, and F(ab′)₂, an Fab′, an scFv, or an sdAb, such as a V_{H}H or a V_{NAR}. In some embodiments, the second antigen-binding portion may bind to human serum albumin, e.g., for the purposes of stabilization/half-life extension.

In some embodiment, the multivalent antibody may comprise a combination of two or more of: a monoclonal antibody, an antigen-binding fragment thereof, an Fab, an F(ab′)₂, and Fab′, and an scFV.

In one embodiment, the multivalent antibody is a trispecific antibody that additionally comprises a third antigen-binding portion.

In some embodiments, the second antigen binding portion comprises a monoclonal antibody, an Fab, and F(ab′)₂, and Fab′, an sdFv, or an sdAb, such as a V_{H}H or a V_{NAR}. In some embodiments, the third antigen binding portion comprises, independently, a monoclonal antibody, an Fab, and F(ab′)₂, and Fab′, an sdFv, or an sdAb, such as a V_{H}H or a V_{NAR}. The second and/or third antigen-binding portion may bind to human serum albumin, e.g., for the purposes of stabilization/half-life extension.

### Bispecific T-Cell Engager

In one embodiment, the multispecific antibody is a bispecific T-cell engager that comprises a first antigen-binding portion comprising the monoclonal antibody, or the antigen-binding fragment thereof, as defined herein, and a second antigen-binding portion. In one embodiment, the second antigen-binding moiety binds specifically to a cell-surface marker.

The term “bispecific T-cell engager”, as used herein, refers to a recombinant bispecific protein that has two linked variable regions from two different antibodies, one targeting a cell-surface molecule on T cells (for example, CD3E), and the other targeting antigens on the surface of disease cells, typically malignant cells. For example a bispecific T-cell engager may comprises a monoclonal antibody, or an antigen-binding fragment thereof, as defined herein and an scFvs. A bispecific T-cell engager may comprise a monoclonal antibody, or an antigen-binding fragment thereof, as defined herein and a second antibody. The two are typically linked together by a short flexible linker such as GlySer linker. By binding to tumor antigens and T cells simultaneously, bispecific T-cell engagers mediate T-cell responses and killing of tumor cells. The T-cell/target cell adherence facilitated by a bispecific T-cell engager is independent of MHC haplotype.

A “cell surface marker” is a molecule expressed at the surface of the cell that is particular to (or enriched in) a cell type, and that is capable of being bound or recognized by an antigen-binding portion.

In one embodiment, the cell surface marker may be a disease-associated marker. In one embodiment, the disease-associated marker may be a tumour-associated antigen.

By “tumour-associated antigen” is meant an antigen, the expression of which is relatively restricted to tumor cells.

In one embodiment, the cell surface marker may be a disease-specific marker. In one embodiment, the disease-specific marker may be a tumour-specific antigen.

By “tumour-specific antigen” is mean an antigen that is uniquely expressed by tumor cells.

In some embodiments, the second antigen binding portion may comprise a monoclonal antibody, an Fab, and F(ab′)₂, an Fab′, an scFv, or an sdAb, such as a V_{H}H or a V_{NAR}. In one embodiment, the first antigen-binding portion is an scFv.

In one embodiment, the cell-surface marker is human EGFR, CD22, or BCMA. In one embodiment, the cell-surface marker is mesothelin, MUC1, EGFRvIII, CD19, CD20, CAIX, FAP, or HER2.

In one embodiment, the multivalent antibody comprises in N-terminal to C-terminal direction:
the second antigen-binding portion,
an amino acid linker, and
the first antigen-binding portion.

In one embodiment, the multivalent antibody further comprises an N-terminal signal peptide.

A “signal peptide”, as referred to herein allows the nascent protein to be directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule.

In one embodiment, the signal peptide is a signal peptide from human CD28. In one embodiment, the signal peptide from human CD28 comprises SEQ ID NO: 156. In one embodiment, the signal peptide is at least 80% identical to SEQ ID NO: 156. In one embodiment, the signal peptide is at least 90% identical to SEQ ID NO: 156. In one embodiment, the signal peptide is at least 95% identical to SEQ ID NO: 156. In one embodiment, the signal peptide is at least 98% identical to SEQ ID NO: 156.

By “amino acid linker”, in this context, will be understood a sequence of sufficient length, flexibility, and composition to permit the bispecific T-cell engager to be properly functional an engage with both targets. An appropriate linker could be readily selected and tested.

In one embodiment, the amino acid linker comprises GGGGS (SEQ ID NO: 164). In one embodiment, the amino acid linker comprises GGGGSGGGGSGGGGS (SEQ ID ON: 165). In some embodiments, the amino acid linker comprises a human CD8 hinge domain (SEQ ID NO: 158).

In one embodiment, the multivalent antibody has an amino acid sequence comprising SEQ ID No: 39.

In one embodiment, the multivalent antibody has an amino acid sequence comprising SEQ ID No: 40.

In one embodiment, the multivalent antibody has an amino acid sequence comprising SEQ ID No: 43.

In one embodiment, the isolated or purified monoclonal antibody, or the antigen-binding fragment thereof comprises CDRH1, CDRH2, and CDRH3 sequences of SEQ ID NO: 1 and CDRL1, CDRL2, and CDRL3 sequence of SEQ ID NO: 2.

In one embodiment, the isolated or purified monoclonal antibody, or the antigen-binding fragment thereof comprises CDRH1, CDRH2, and CDRH3 sequences of SEQ ID NO: 3 and CDRL1, CDRL2, and CDRL3 sequence of SEQ ID NO: 4.

In one embodiment, the isolated or purified monoclonal antibody, or the antigen-binding fragment thereof comprises CDRH1, CDRH2, and CDRH3 sequences of SEQ ID NO: 5 and CDRL1, CDRL2, and CDRL3 sequence of SEQ ID NO: 6.

In one embodiment, the isolated or purified monoclonal antibody, or the antigen-binding fragment thereof comprises CDRH1, CDRH2, and CDRH3 sequences of SEQ ID NO: 7 and CDRL1, CDRL2, and CDRL3 sequence of SEQ ID NO: 8.

In one embodiment, the isolated or purified monoclonal antibody, or the antigen-binding fragment thereof comprises CDRH1, CDRH2, and CDRH3 sequences of SEQ ID NO: 9 and CDRL1, CDRL2, and CDRL3 sequence of SEQ ID NO: 10.

In one embodiment, the isolated or purified monoclonal antibody, or the antigen-binding fragment thereof comprises CDRH1, CDRH2, and CDRH3 sequences of SEQ ID NO: 11 and CDRL1, CDRL2, and CDRL3 sequence of SEQ ID NO: 12.

In one embodiment, the isolated or purified monoclonal antibody, or the antigen-binding fragment thereof comprises CDRH1, CDRH2, and CDRH3 sequences of SEQ ID NO: 13 and CDRL1, CDRL2, and CDRL3 sequence of SEQ ID NO: 14.

In one embodiment, the isolated or purified monoclonal antibody, or the antigen-binding fragment thereof comprises CDRH1, CDRH2, and CDRH3 sequences of SEQ ID NO: 15 and CDRL1, CDRL2, and CDRL3 sequence of SEQ ID NO: 16.

In one embodiment, the isolated or purified monoclonal antibody, or the antigen-binding fragment thereof comprises CDRH1, CDRH2, and CDRH3 sequences of SEQ ID NO: 17 and CDRL1, CDRL2, and CDRL3 sequence of SEQ ID NO: 18.

In one embodiment, the isolated or purified monoclonal antibody, or the antigen-binding fragment thereof comprises CDRH1, CDRH2, and CDRH3 sequences of SEQ ID NO: 19 and CDRL1, CDRL2, and CDRL3 sequence of SEQ ID NO: 20.

In one embodiment, the isolated or purified monoclonal antibody, or the antigen-binding fragment thereof comprises CDRH1, CDRH2, and CDRH3 sequences of SEQ ID NO: 21 and CDRL1, CDRL2, and CDRL3 sequence of SEQ ID NO: 22.

In one embodiment, the isolated or purified monoclonal antibody, or the antigen-binding fragment thereof comprises CDRH1, CDRH2, and CDRH3 sequences of SEQ ID NO: 23 and CDRL1, CDRL2, and CDRL3 sequence of SEQ ID NO: 24.

In one embodiment, the isolated or purified monoclonal antibody, or the antigen-binding fragment thereof comprises CDRH1, CDRH2, and CDRH3 sequences of SEQ ID NO: 25 and CDRL1, CDRL2, and CDRL3 sequence of SEQ ID NO: 26.

In one embodiment, the isolated or purified monoclonal antibody, or the antigen-binding fragment thereof comprises CDRH1, CDRH2, and CDRH3 sequences of SEQ ID NO: 27 and CDRL1, CDRL2, and CDRL3 sequence of SEQ ID NO: 28.

In one embodiment, the isolated or purified monoclonal antibody, or the antigen-binding fragment thereof comprises CDRH1, CDRH2, and CDRH3 sequences of SEQ ID NO: 29 and CDRL1, CDRL2, and CDRL3 sequence of SEQ ID NO: 30.

In one embodiment, the isolated or purified monoclonal antibody, or the antigen-binding fragment thereof comprises CDRH1, CDRH2, and CDRH3 sequences of SEQ ID NO: 31 and CDRL1, CDRL2, and CDRL3 sequence of SEQ ID NO: 32.

In one embodiment, the isolated or purified monoclonal antibody, or the antigen-binding fragment thereof comprises CDRH1, CDRH2, and CDRH3 sequences of SEQ ID NO: 33 and CDRL1, CDRL2, and CDRL3 sequence of SEQ ID NO: 34.

In one embodiment, the isolated or purified monoclonal antibody, or the antigen-binding fragment thereof comprises:
a CDRH1 amino acid sequence of SEQ ID NO: 48,
a CDRH2 amino acid sequence of SEQ ID NO: 49,
a CDRH3 amino acid sequence of SEQ ID NO: 50,
a CDRL1 amino acid sequence of SEQ ID NO: 51,
a CDRL2 amino acid sequence of SEQ ID NO: 52, and
a CDRL3 amino acid sequence of SEQ ID NO: 53.

In one embodiment, the isolated or purified monoclonal antibody, or the antigen-binding fragment thereof comprises:
a CDRH1 amino acid sequence of SEQ ID NO: 54,
a CDRH2 amino acid sequence of SEQ ID NO: 55,
a CDRH3 amino acid sequence of SEQ ID NO: 56,
a CDRL1 amino acid sequence of SEQ ID NO: 57,
a CDRL2 amino acid sequence of SEQ ID NO: 58, and
a CDRL3 amino acid sequence of SEQ ID NO: 59.

In one embodiment, the isolated or purified monoclonal antibody, or the antigen-binding fragment thereof comprises:
a CDRH1 amino acid sequence of SEQ ID NO: 60,
a CDRH2 amino acid sequence of SEQ ID NO: 61,
a CDRH3 amino acid sequence of SEQ ID NO: 62,
a CDRL1 amino acid sequence of SEQ ID NO: 63,
a CDRL2 amino acid sequence of SEQ ID NO: 64, and
a CDRL3 amino acid sequence of SEQ ID NO: 65.

In one embodiment, the isolated or purified monoclonal antibody, or the antigen-binding fragment thereof comprises:
a CDRH1 amino acid sequence of SEQ ID NO: 66,
a CDRH2 amino acid sequence of SEQ ID NO: 67,
a CDRH3 amino acid sequence of SEQ ID NO: 68,
a CDRL1 amino acid sequence of SEQ ID NO: 69,
a CDRL2 amino acid sequence of SEQ ID NO: 70, and
a CDRL3 amino acid sequence of SEQ ID NO: 71.

In one embodiment, the isolated or purified monoclonal antibody, or the antigen-binding fragment thereof comprises:
a CDRH1 amino acid sequence of SEQ ID NO: 72,
a CDRH2 amino acid sequence of SEQ ID NO: 73,
a CDRH3 amino acid sequence of SEQ ID NO: 74,
a CDRL1 amino acid sequence of SEQ ID NO: 75,
a CDRL2 amino acid sequence of SEQ ID NO: 76, and
a CDRL3 amino acid sequence of SEQ ID NO: 77.

In one embodiment, the isolated or purified monoclonal antibody, or the antigen-binding fragment thereof comprises:
a CDRH1 amino acid sequence of SEQ ID NO: 78,
a CDRH2 amino acid sequence of SEQ ID NO: 79,
a CDRH3 amino acid sequence of SEQ ID NO: 80,
a CDRL1 amino acid sequence of SEQ ID NO: 81,
a CDRL2 amino acid sequence of SEQ ID NO: 82, and
a CDRL3 amino acid sequence of SEQ ID NO: 83.

In one embodiment, the isolated or purified monoclonal antibody, or the antigen-binding fragment thereof comprises:
a CDRH1 amino acid sequence of SEQ ID NO: 84,
a CDRH2 amino acid sequence of SEQ ID NO: 85,
a CDRH3 amino acid sequence of SEQ ID NO: 86,
a CDRL1 amino acid sequence of SEQ ID NO: 87,
a CDRL2 amino acid sequence of SEQ ID NO: 88, and
a CDRL3 amino acid sequence of SEQ ID NO: 89.

In one embodiment, the isolated or purified monoclonal antibody, or the antigen-binding fragment thereof comprises:
a CDRH1 amino acid sequence of SEQ ID NO: 90,
a CDRH2 amino acid sequence of SEQ ID NO: 91,
a CDRH3 amino acid sequence of SEQ ID NO: 92,
a CDRL1 amino acid sequence of SEQ ID NO: 93,
a CDRL2 amino acid sequence of SEQ ID NO: 94, and
a CDRL3 amino acid sequence of SEQ ID NO: 95.

In one embodiment, the isolated or purified monoclonal antibody, or the antigen-binding fragment thereof comprises:
a CDRH1 amino acid sequence of SEQ ID NO: 96,
a CDRH2 amino acid sequence of SEQ ID NO: 97,
a CDRH3 amino acid sequence of SEQ ID NO: 98,
a CDRL1 amino acid sequence of SEQ ID NO: 99,
a CDRL2 amino acid sequence of SEQ ID NO: 100, and
a CDRL3 amino acid sequence of SEQ ID NO: 101.

In one embodiment, the isolated or purified monoclonal antibody, or the antigen-binding fragment thereof comprises:
a CDRH1 amino acid sequence of SEQ ID NO: 102,
a CDRH2 amino acid sequence of SEQ ID NO: 103,
a CDRH3 amino acid sequence of SEQ ID NO: 104,
a CDRL1 amino acid sequence of SEQ ID NO: 105,
a CDRL2 amino acid sequence of SEQ ID NO: 106, and
a CDRL3 amino acid sequence of SEQ ID NO: 107.

In one embodiment, the isolated or purified monoclonal antibody, or the antigen-binding fragment thereof comprises:
a CDRH1 amino acid sequence of SEQ ID NO: 108,
a CDRH2 amino acid sequence of SEQ ID NO: 109,
a CDRH3 amino acid sequence of SEQ ID NO: 110,
a CDRL1 amino acid sequence of SEQ ID NO: 111,
a CDRL2 amino acid sequence of SEQ ID NO: 112, and
a CDRL3 amino acid sequence of SEQ ID NO: 113.

In one embodiment, the isolated or purified monoclonal antibody, or the antigen-binding fragment thereof comprises:
a CDRH1 amino acid sequence of SEQ ID NO: 114,
a CDRH2 amino acid sequence of SEQ ID NO: 115,
a CDRH3 amino acid sequence of SEQ ID NO: 116,
a CDRL1 amino acid sequence of SEQ ID NO: 117,
a CDRL2 amino acid sequence of SEQ ID NO: 118, and
a CDRL3 amino acid sequence of SEQ ID NO: 119.

In one embodiment, the isolated or purified monoclonal antibody, or the antigen-binding fragment thereof comprises:
a CDRH1 amino acid sequence of SEQ ID NO: 120,
a CDRH2 amino acid sequence of SEQ ID NO: 121,
a CDRH3 amino acid sequence of SEQ ID NO: 122,
a CDRL1 amino acid sequence of SEQ ID NO: 123,
a CDRL2 amino acid sequence of SEQ ID NO: 124, and
a CDRL3 amino acid sequence of SEQ ID NO: 125.

In one embodiment, the isolated or purified monoclonal antibody, or the antigen-binding fragment thereof comprises:
a CDRH1 amino acid sequence of SEQ ID NO: 126,
a CDRH2 amino acid sequence of SEQ ID NO: 127,
a CDRH3 amino acid sequence of SEQ ID NO: 128,
a CDRL1 amino acid sequence of SEQ ID NO: 129,
a CDRL2 amino acid sequence of SEQ ID NO: 130, and
a CDRL3 amino acid sequence of SEQ ID NO: 131.

In one embodiment, the isolated or purified monoclonal antibody, or the antigen-binding fragment thereof comprises:
a CDRH1 amino acid sequence of SEQ ID NO: 132,
a CDRH2 amino acid sequence of SEQ ID NO: 133,
a CDRH3 amino acid sequence of SEQ ID NO: 134,
a CDRL1 amino acid sequence of SEQ ID NO: 135,
a CDRL2 amino acid sequence of SEQ ID NO: 136, and
a CDRL3 amino acid sequence of SEQ ID NO: 137.

In one embodiment, the isolated or purified monoclonal antibody, or the antigen-binding fragment thereof comprises:
a CDRH1 amino acid sequence of SEQ ID NO: 138,
a CDRH2 amino acid sequence of SEQ ID NO: 139,
a CDRH3 amino acid sequence of SEQ ID NO: 140,
a CDRL1 amino acid sequence of SEQ ID NO: 141,
a CDRL2 amino acid sequence of SEQ ID NO: 142, and
a CDRL3 amino acid sequence of SEQ ID NO: 143.

In one embodiment, the isolated or purified monoclonal antibody, or the antigen-binding fragment thereof comprises:
a CDRH1 amino acid sequence of SEQ ID NO: 144,
a CDRH2 amino acid sequence of SEQ ID NO: 145,
a CDRH3 amino acid sequence of SEQ ID NO: 146,
a CDRL1 amino acid sequence of SEQ ID NO: 147,
a CDRL2 amino acid sequence of SEQ ID NO: 148, and
a CDRL3 amino acid sequence of SEQ ID NO: 149.

In one embodiment, the first antigen-binding portion comprises SEQ ID NO: 37.

In one embodiment, the second antigen-biding portion binds to EGFRvIII. In one embodiment, the multivalent antibody has the sequence of SEQ ID No: 40.

### Recombinant Nucleic Acid Molecules

In aspect, there is provided a recombinant nucleic acid molecule encoding the multivalent antibody as defined herein. In one embodiment, there is provided a ventor comprising the nucleic acid described herein. In one embodiment, the nucleic acid is DNA. In one embodiment, the nucleic acid is RNA. In one embodiment, the RNA is an mRNA.

### Compositions

In one aspect, there is provided a composition comprising a multivalent antibody as defined herein; together with an acceptable excipient, diluent or carrier. In one embodiment, the composition comprises a bispecific T-cell engager as herein defined. In one embodiment the composition is a pharmaceutical composition, and the excipient, diluent or carrier is a pharmaceutically acceptable excipient, diluent or carrier.

### Uses & Methods

In one aspect, there is provided a use of the multivalent antibody as defined herein for killing a cell. In one embodiment, the cell is a disease cell. In one embodiment, the cell is a undesired cell. In one embodiment, the cell is a fibrotic cell. Targeting fibrotic cells, in some embodiments, may be used for treatment of heart disease. In one embodiment, the cell is a senescent cell. Targeting senescent cells, in some embodiments, may be used for treatment of aging.

In one aspect, there is provided a use of the multivalent antibody as defined herein for preparation of a medicament for killing a cell. In one embodiment, the cell is a disease cell. In one embodiment, the cell is a undesired cell. In one embodiment, the cell is a fibrotic cell. Targeting fibrotic cells, in some embodiments, may be used for treatment of heart disease. In one embodiment, the cell is a senescent cell. Targeting senescent cells, in some embodiments, may be used for treatment of aging.

In one aspect, there is provided the multivalent antibody as defined herein for use in killing a cell. In one embodiment, the cell is a disease cell. In one embodiment, the cell is a undesired cell. In one embodiment, the cell is a fibrotic cell. Targeting fibrotic cells, in some embodiments, may be used for treatment of heart disease. In one embodiment, the cell is a senescent cell. Targeting senescent cells, in some embodiments, may be used for treatment of aging.

In one aspect, there is provided a method of killing a cell comprising contacting the cell with the multivalent antibody as defined herein. In one embodiment, the cell is a disease cell. In one embodiment, the cell is a undesired cell. In one embodiment, the cell is a fibrotic cell. Targeting fibrotic cells, in some embodiments, may be used for treatment of heart disease. In one embodiment, the cell is a senescent cell. Targeting senescent cells, in some embodiments, may be used for treatment of aging.

In one aspect, there is provided a use of the multivalent antibody as defined herein for killing a tumour cell.

In one aspect, there is provided a use of the multivalent antibody as defined herein for preparation of a medicament for killing a tumour cell.

In one aspect, there is provided the multivalent antibody as defined herein for use in killing a tumour cell.

In one aspect, there is provided a method of killing a tumour cell comprising contacting the tumour cell with the multivalent antibody as defined herein.

In one aspect, there is provided a use of the multivalent antibody as defined herein for treatment of a cancer. In one embodiment, the cancer is a leukemia, lymphoma, multiple myeloma, lung cancer, pancreatic cancer, gastric cancer, colon cancer, or brain cancer.

In one aspect, there is provided a use of the multivalent antibody as defined herein for preparation of a medicament for treatment of a cancer. In one embodiment, the cancer is a leukemia, lymphoma, multiple myeloma, lung cancer, pancreatic cancer, gastric cancer, colon cancer, or brain cancer.

In one aspect, there is provided the multivalent antibody as defined herein for use in treatment of a cancer. In one embodiment, the cancer is a leukemia, lymphoma, multiple myeloma, lung cancer, pancreatic cancer, gastric cancer, colon cancer, or brain cancer.

In one aspect, there is provided a method of treating a cancer in subject comprising administering to the subject the multivalent antibody as defined herein. In one embodiment, the cancer is a leukemia, lymphoma, multiple myeloma, lung cancer, pancreatic cancer, gastric cancer, colon cancer, or brain cancer.

### Recombinant Polypeptides Comprising a Sequon

In one embodiment, the recombinant polypeptide described herein further comprises an artificial glycosylation sequon. In one embodiment the artificial glycosylation sequon comprises the amino acid sequence of SEQ ID No: 157.

In one embodiment, the one or more monoclonal antibody, or antigen-binding fragment thereof comprises:
a CDRH1 amino acid sequence as set forth in SEQ ID NO: 60,
a CDRH2 amino acid sequence as set forth in SEQ ID NO: 61,
a CDRH3 amino acid sequence as set forth in SEQ ID NO: 62,
a CDRL1 amino acid sequence as set forth in SEQ ID NO: 63,
a CDRL2 amino acid sequence as set forth in SEQ ID NO: 64, and
a CDRL3 amino acid sequence as set forth in SEQ ID NO: 65 (from F322-1E2); or
a CDRH1 amino acid sequence as set forth in SEQ ID NO: 72,
a CDRH2 amino acid sequence as set forth in SEQ ID NO: 73,
a CDRH3 amino acid sequence as set forth in SEQ ID NO: 74,
a CDRL1 amino acid sequence as set forth in SEQ ID NO: 75,
a CDRL2 amino acid sequence as set forth in SEQ ID NO: 76, and
a CDRL3 amino acid sequence as set forth in SEQ ID NO: 77 (from F322-4D6-2);

In one embodiment, the antigen-binding fragment comprises:
a heavy chain amino acid sequence of SEQ ID No: 45 and a light chain amino acid sequence of SEQ ID No: 44 (F322-1E2 Fab); or
a heavy chain amino acid sequence of SEQ ID No: 47 and a light chain amino acid sequence of SEQ ID No: 46 (F322-4D6 Fab).

In one aspect, there is provided a conjugated lipid nanoparticle, which is conjugated to the recombinant polypeptide comprising the sequon, as defined herein.

In one embodiment, the conjugated lipid nanoparticle further comprises comprising a cargo molecule within the lipid nanoparticle.

In one embodiment, the cargo molecule comprises a nucleic acid.

In one embodiment, the nucleic acid is an mRNA.

In one aspect, there is provided a use of the conjugated lipid nanoparticle as defined herein for delivery of the cargo molecule to a T cell.

In one aspect, there is provided a method of delivering a cargo molecule to a T cell comprising contacting the T cell with the conjugated lipid nanoparticle as defined herein.

### Chimeric Antibody Receptors & Related Embodiments

In one aspect, there is provided a chimeric antibody receptor (CAR), which binds to human CD3, comprising the isolated or purified monoclonal antibody, or the antigen-binding fragment thereof, as defined herein.

Chimeric antigen receptors” are receptor proteins engineered to give T cells the new ability to target a specific protein. The receptors are chimeric because they combine both antigen-binding and T-cell activating functions into a single receptor (see Stoiber et al. Limitations in the Design of Chimeric Antigen Receptors for Cancer Therapy. Cells. 2012; 8(5): 472 and van der Stegen et al. The pharmacology of second-generation chimeric antigen receptors. Nat Rev Drug Discov. 2019; 14(7): 499-509).

In one embodiment, the CAR comprises in N-terminal to C-terminal direction:
an CD3 binding domain comprising isolated or purified monoclonal antibody, or the antigen-binding fragment thereof as defined in any one of claims 1 to 29,
a polypeptide hinge,
a transmembrane domain, and
a cytoplasmic domain comprising a signaling domain, preferably wherein the cytoplasmic domain further comprises a co-stimulatory domain.

The term “polypeptide hinge” used herein generally means any oligo- or polypeptide that functions to link the extracellular ligand-binding domain to the transmembrane domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence.

In one embodiment, the polypeptide hinge comprises a human CD8 hinge domain or a fragment thereof. In one embodiment, the polypeptide hinge comprises SEQ ID NO: 158.

The term “transmembrane domain” indicates a polypeptide having the ability to span a cell membrane and thereby link the extracellular portion of the CAR (which comprises the BCMA-binding portion) to the intracellular portion responsible for signaling. Commonly used transmembrane domains for CARs have been derived from CD4, CD8α, CD28 and CD3.

In one embodiment, the transmembrane domain is a CD28 transmembrane domain. In one embodiment, the CD28 transmembrane domain comprises SEQ ID NO: 159. In one embodiment, the transmembrane domain is at least 80% identical to SEQ ID NO: 159. In one embodiment, the transmembrane domain is at least 90% identical to SEQ ID NO: 159. In one embodiment, the transmembrane domain is at least 95% identical to SEQ ID NO: 159. In one embodiment, the transmembrane domain is at least 98% identical to SEQ ID NO: 159.

The term “cytoplasmic domain” (also termed a “signal transduction domain”) refers to the intracellular portion of the CAR that is responsible for intracellular signaling following the binding of extracellular ligand binding domain to the target resulting in the activation of the immune cell and immune response. In other words, cytoplasmic domain is responsible for the activation of at least one of the normal effector functions of the immune cell in which the CAR is expressed. For example, the effector function of a T cell can be a cytolytic activity or helper activity including the secretion of cytokines. Thus, the term “cytoplasmic domain” refers to the portion of a protein which transduces the effector signal and directs the cell to perform a specialized function. It is common for such cytoplasmic domains to comprise a co-stimulatory domain in addition to a signaling domain.

The term “signaling domain” refers to the portion of a protein which transduces the effector signal and directs the cell to perform a specialized function. Examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transducing domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Non-limiting examples of signaling domains used in the invention can include those derived from TCRzeta, common FcR gamma (FCERIG), Fcgamma RIIa, FcRbeta (Fc Epsilon Rib), FcRepsilon, CD3 zeta, CD3gamma, CD3delta, CD3epsilon, CD5, CD22, CD79a, CD79b, CD66d, DAP10, or DAP12. In a preferred embodiment, the signaling transducing domain of the CAR can comprise the CD3zeta signaling domain.

In one embodiment, the signaling domain is a CD3-zeta signaling domain. In one embodiment, the CD3-zeta signaling domain comprises SEQ ID NO: 160. In one embodiment, the signaling domain is at least 80% identical to SEQ ID NO: 160. In one embodiment, the signaling domain is at least 90% identical to SEQ ID NO: 160. In one embodiment, the signaling domain is at least 95% identical to SEQ ID NO: 160. In one embodiment, the signaling domain is at least 98% identical to SEQ ID NO: 160.

The term “co-stimulatory domain” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, 4-1 BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDI Id, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D or a combination thereof.

In one embodiment, the co-stimulatory domain is a 4-1BB co-stimulatory domain. In one embodiment, the 4-1BB signal transduction domain comprises SEQ ID NO: 161. In one embodiment, the co-stimulatory domain is at least 80% identical to SEQ ID NO: 161. In one embodiment, the co-stimulatory domain is at least 90% identical to SEQ ID NO: 161. In one embodiment, the co-stimulatory domain is at least 95% identical to SEQ ID NO: 161. In one embodiment, the co-stimulatory domain is at least 98% identical to SEQ ID NO: 161.

In one embodiment, CAR further comprises a flexible amino acid linker between the CD3 binding domain and the polypeptide hinge. In one embodiment, the amino acid linker comprises SEQ ID NO: 162. In one embodiment, the amino acid linker is at least 80% identical to SEQ ID NO: 162. In one embodiment, the amino acid linker is at least 90% identical to SEQ ID NO: 162. In one embodiment, the amino acid linker is at least 95% identical to SEQ ID NO: 162. In one embodiment, the amino acid linker is at least 98% identical to SEQ ID NO: 162.

In one embodiment, the CAR further comprises a signal peptide.

In one embodiment, the signal peptide is a signal peptide from human CD28. In one embodiment, the signal peptide from human CD28 comprises SEQ ID NO: 163. In one embodiment, the signal peptide is at least 80% identical to SEQ ID NO: 163. In one embodiment, the signal peptide is at least 90% identical to SEQ ID NO: 163. In one embodiment, the signal peptide is at least 95% identical to SEQ ID NO: 163. In one embodiment, the signal peptide is at least 98% identical to SEQ ID NO: 163.

In one embodiment, the isolated or purified monoclonal antibody, or the antigen-binding fragment thereof comprises CDRH1, CDRH2, and CDRH3 sequences of SEQ ID NO: 1 and CDRL1, CDRL2, and CDRL3 sequence of SEQ ID NO: 2.

In one embodiment, the isolated or purified monoclonal antibody, or the antigen-binding fragment thereof comprises CDRH1, CDRH2, and CDRH3 sequences of SEQ ID NO: 3 and CDRL1, CDRL2, and CDRL3 sequence of SEQ ID NO: 4.

In one embodiment, the isolated or purified monoclonal antibody, or the antigen-binding fragment thereof comprises CDRH1, CDRH2, and CDRH3 sequences of SEQ ID NO: 5 and CDRL1, CDRL2, and CDRL3 sequence of SEQ ID NO: 6.

In one embodiment, the isolated or purified monoclonal antibody, or the antigen-binding fragment thereof comprises CDRH1, CDRH2, and CDRH3 sequences of SEQ ID NO: 7 and CDRL1, CDRL2, and CDRL3 sequence of SEQ ID NO: 8.

In one embodiment, the isolated or purified monoclonal antibody, or the antigen-binding fragment thereof comprises CDRH1, CDRH2, and CDRH3 sequences of SEQ ID NO: 9 and CDRL1, CDRL2, and CDRL3 sequence of SEQ ID NO: 10.

In one embodiment, the isolated or purified monoclonal antibody, or the antigen-binding fragment thereof comprises CDRH1, CDRH2, and CDRH3 sequences of SEQ ID NO: 11 and CDRL1, CDRL2, and CDRL3 sequence of SEQ ID NO: 12.

In one embodiment, the isolated or purified monoclonal antibody, or the antigen-binding fragment thereof comprises CDRH1, CDRH2, and CDRH3 sequences of SEQ ID NO: 13 and CDRL1, CDRL2, and CDRL3 sequence of SEQ ID NO: 14.

In one embodiment, the isolated or purified monoclonal antibody, or the antigen-binding fragment thereof comprises CDRH1, CDRH2, and CDRH3 sequences of SEQ ID NO: 15 and CDRL1, CDRL2, and CDRL3 sequence of SEQ ID NO: 16.

In one embodiment, the isolated or purified monoclonal antibody, or the antigen-binding fragment thereof comprises CDRH1, CDRH2, and CDRH3 sequences of SEQ ID NO: 17 and CDRL1, CDRL2, and CDRL3 sequence of SEQ ID NO: 18.

In one embodiment, the isolated or purified monoclonal antibody, or the antigen-binding fragment thereof comprises CDRH1, CDRH2, and CDRH3 sequences of SEQ ID NO: 19 and CDRL1, CDRL2, and CDRL3 sequence of SEQ ID NO: 20.

In one embodiment, the isolated or purified monoclonal antibody, or the antigen-binding fragment thereof comprises CDRH1, CDRH2, and CDRH3 sequences of SEQ ID NO: 21 and CDRL1, CDRL2, and CDRL3 sequence of SEQ ID NO: 22.

In one embodiment, the isolated or purified monoclonal antibody, or the antigen-binding fragment thereof comprises CDRH1, CDRH2, and CDRH3 sequences of SEQ ID NO: 23 and CDRL1, CDRL2, and CDRL3 sequence of SEQ ID NO: 24.

In one embodiment, the isolated or purified monoclonal antibody, or the antigen-binding fragment thereof comprises CDRH1, CDRH2, and CDRH3 sequences of SEQ ID NO: 25 and CDRL1, CDRL2, and CDRL3 sequence of SEQ ID NO: 26.

In one embodiment, the isolated or purified monoclonal antibody, or the antigen-binding fragment thereof comprises CDRH1, CDRH2, and CDRH3 sequences of SEQ ID NO: 27 and CDRL1, CDRL2, and CDRL3 sequence of SEQ ID NO: 28.

In one embodiment, the isolated or purified monoclonal antibody, or the antigen-binding fragment thereof comprises CDRH1, CDRH2, and CDRH3 sequences of SEQ ID NO: 29 and CDRL1, CDRL2, and CDRL3 sequence of SEQ ID NO: 30.

In one embodiment, the isolated or purified monoclonal antibody, or the antigen-binding fragment thereof comprises CDRH1, CDRH2, and CDRH3 sequences of SEQ ID NO: 31 and CDRL1, CDRL2, and CDRL3 sequence of SEQ ID NO: 32.

In one embodiment, the isolated or purified monoclonal antibody, or the antigen-binding fragment thereof comprises CDRH1, CDRH2, and CDRH3 sequences of SEQ ID NO: 33 and CDRL1, CDRL2, and CDRL3 sequence of SEQ ID NO: 34.

In one embodiment, the isolated or purified monoclonal antibody, or the antigen-binding fragment thereof comprises:
a CDRH1 amino acid sequence of SEQ ID NO: 48,
a CDRH2 amino acid sequence of SEQ ID NO: 49,
a CDRH3 amino acid sequence of SEQ ID NO: 50,
a CDRL1 amino acid sequence of SEQ ID NO: 51,
a CDRL2 amino acid sequence of SEQ ID NO: 52, and
a CDRL3 amino acid sequence of SEQ ID NO: 53.

In one embodiment, the isolated or purified monoclonal antibody, or the antigen-binding fragment thereof comprises:
a CDRH1 amino acid sequence of SEQ ID NO: 54,
a CDRH2 amino acid sequence of SEQ ID NO: 55,
a CDRH3 amino acid sequence of SEQ ID NO: 56,
a CDRL1 amino acid sequence of SEQ ID NO: 57,
a CDRL2 amino acid sequence of SEQ ID NO: 58, and
a CDRL3 amino acid sequence of SEQ ID NO: 59.

In one embodiment, the isolated or purified monoclonal antibody, or the antigen-binding fragment thereof comprises:
a CDRH1 amino acid sequence of SEQ ID NO: 60,
a CDRH2 amino acid sequence of SEQ ID NO: 61,
a CDRH3 amino acid sequence of SEQ ID NO: 62,
a CDRL1 amino acid sequence of SEQ ID NO: 63,
a CDRL2 amino acid sequence of SEQ ID NO: 64, and
a CDRL3 amino acid sequence of SEQ ID NO: 65.

In one embodiment, the isolated or purified monoclonal antibody, or the antigen-binding fragment thereof comprises:
a CDRH1 amino acid sequence of SEQ ID NO: 66,
a CDRH2 amino acid sequence of SEQ ID NO: 67,
a CDRH3 amino acid sequence of SEQ ID NO: 68,
a CDRL1 amino acid sequence of SEQ ID NO: 69,
a CDRL2 amino acid sequence of SEQ ID NO: 70, and
a CDRL3 amino acid sequence of SEQ ID NO: 71.

In one embodiment, the isolated or purified monoclonal antibody, or the antigen-binding fragment thereof comprises:
a CDRH1 amino acid sequence of SEQ ID NO: 72,
a CDRH2 amino acid sequence of SEQ ID NO: 73,
a CDRH3 amino acid sequence of SEQ ID NO: 74,
a CDRL1 amino acid sequence of SEQ ID NO: 75,
a CDRL2 amino acid sequence of SEQ ID NO: 76, and
a CDRL3 amino acid sequence of SEQ ID NO: 77.

In one embodiment, the isolated or purified monoclonal antibody, or the antigen-binding fragment thereof comprises:
a CDRH1 amino acid sequence of SEQ ID NO: 78,
a CDRH2 amino acid sequence of SEQ ID NO: 79,
a CDRH3 amino acid sequence of SEQ ID NO: 80,
a CDRL1 amino acid sequence of SEQ ID NO: 81,
a CDRL2 amino acid sequence of SEQ ID NO: 82, and
a CDRL3 amino acid sequence of SEQ ID NO: 83.

In one embodiment, the isolated or purified monoclonal antibody, or the antigen-binding fragment thereof comprises:
a CDRH1 amino acid sequence of SEQ ID NO: 84,
a CDRH2 amino acid sequence of SEQ ID NO: 85,
a CDRH3 amino acid sequence of SEQ ID NO: 86,
a CDRL1 amino acid sequence of SEQ ID NO: 87,
a CDRL2 amino acid sequence of SEQ ID NO: 88, and
a CDRL3 amino acid sequence of SEQ ID NO: 89.

In one embodiment, the isolated or purified monoclonal antibody, or the antigen-binding fragment thereof comprises:
a CDRH1 amino acid sequence of SEQ ID NO: 90,
a CDRH2 amino acid sequence of SEQ ID NO: 91,
a CDRH3 amino acid sequence of SEQ ID NO: 92,
a CDRL1 amino acid sequence of SEQ ID NO: 93,
a CDRL2 amino acid sequence of SEQ ID NO: 94, and
a CDRL3 amino acid sequence of SEQ ID NO: 95.

In one embodiment, the isolated or purified monoclonal antibody, or the antigen-binding fragment thereof comprises:
a CDRH1 amino acid sequence of SEQ ID NO: 96,
a CDRH2 amino acid sequence of SEQ ID NO: 97,
a CDRH3 amino acid sequence of SEQ ID NO: 98,
a CDRL1 amino acid sequence of SEQ ID NO: 99,
a CDRL2 amino acid sequence of SEQ ID NO: 100, and
a CDRL3 amino acid sequence of SEQ ID NO: 101.

In one embodiment, the isolated or purified monoclonal antibody, or the antigen-binding fragment thereof comprises:
a CDRH1 amino acid sequence of SEQ ID NO: 102,
a CDRH2 amino acid sequence of SEQ ID NO: 103,
a CDRH3 amino acid sequence of SEQ ID NO: 104,
a CDRL1 amino acid sequence of SEQ ID NO: 105,
a CDRL2 amino acid sequence of SEQ ID NO: 106, and
a CDRL3 amino acid sequence of SEQ ID NO: 107.

In one embodiment, the isolated or purified monoclonal antibody, or the antigen-binding fragment thereof comprises:
a CDRH1 amino acid sequence of SEQ ID NO: 108,
a CDRH2 amino acid sequence of SEQ ID NO: 109,
a CDRH3 amino acid sequence of SEQ ID NO: 110,
a CDRL1 amino acid sequence of SEQ ID NO: 111,
a CDRL2 amino acid sequence of SEQ ID NO: 112, and
a CDRL3 amino acid sequence of SEQ ID NO: 113.

In one embodiment, the isolated or purified monoclonal antibody, or the antigen-binding fragment thereof comprises:
a CDRH1 amino acid sequence of SEQ ID NO: 114,
a CDRH2 amino acid sequence of SEQ ID NO: 115,
a CDRH3 amino acid sequence of SEQ ID NO: 116,
a CDRL1 amino acid sequence of SEQ ID NO: 117,
a CDRL2 amino acid sequence of SEQ ID NO: 118, and
a CDRL3 amino acid sequence of SEQ ID NO: 119.

In one embodiment, the isolated or purified monoclonal antibody, or the antigen-binding fragment thereof comprises:
a CDRH1 amino acid sequence of SEQ ID NO: 120,
a CDRH2 amino acid sequence of SEQ ID NO: 121,
a CDRH3 amino acid sequence of SEQ ID NO: 122,
a CDRL1 amino acid sequence of SEQ ID NO: 123,
a CDRL2 amino acid sequence of SEQ ID NO: 124, and
a CDRL3 amino acid sequence of SEQ ID NO: 125.

In one embodiment, the isolated or purified monoclonal antibody, or the antigen-binding fragment thereof comprises:
a CDRH1 amino acid sequence of SEQ ID NO: 126,
a CDRH2 amino acid sequence of SEQ ID NO: 127,
a CDRH3 amino acid sequence of SEQ ID NO: 128,
a CDRL1 amino acid sequence of SEQ ID NO: 129,
a CDRL2 amino acid sequence of SEQ ID NO: 130, and
a CDRL3 amino acid sequence of SEQ ID NO: 131.

In one embodiment, the isolated or purified monoclonal antibody, or the antigen-binding fragment thereof comprises:
a CDRH1 amino acid sequence of SEQ ID NO: 132,
a CDRH2 amino acid sequence of SEQ ID NO: 133,
a CDRH3 amino acid sequence of SEQ ID NO: 134,
a CDRL1 amino acid sequence of SEQ ID NO: 135,
a CDRL2 amino acid sequence of SEQ ID NO: 136, and
a CDRL3 amino acid sequence of SEQ ID NO: 137.

In one embodiment, the isolated or purified monoclonal antibody, or the antigen-binding fragment thereof comprises:
a CDRH1 amino acid sequence of SEQ ID NO: 138,
a CDRH2 amino acid sequence of SEQ ID NO: 139,
a CDRH3 amino acid sequence of SEQ ID NO: 140,
a CDRL1 amino acid sequence of SEQ ID NO: 141,
a CDRL2 amino acid sequence of SEQ ID NO: 142, and
a CDRL3 amino acid sequence of SEQ ID NO: 143.

In one embodiment, the isolated or purified monoclonal antibody, or the antigen-binding fragment thereof comprises:
a CDRH1 amino acid sequence of SEQ ID NO: 144,
a CDRH2 amino acid sequence of SEQ ID NO: 145,
a CDRH3 amino acid sequence of SEQ ID NO: 146,
a CDRL1 amino acid sequence of SEQ ID NO: 147,
a CDRL2 amino acid sequence of SEQ ID NO: 148, and
a CDRL3 amino acid sequence of SEQ ID NO: 149.

### Nucleic Acids & Vectors

In one aspect, there is provided a recombinant nucleic acid molecule encoding the CAR as defined herein. In one embodiment, the nucleic acid molecule may comprise DNA. In one embodiment, the nucleic acid molecule may comprise RNA. In one embodiment, the nucleic acid molecule may comprise mRNA. In one embodiment, the nucleic acid molecule may comprise any nucleic acids that encode a protein. In one embodiment, nucleic acid is a vector.

In one aspect, there is provided a vector comprising the recombinant nucleic acid molecule as defined herein. In one embodiment, the vector is a viral vector. In one embodiment, the viral vector is a lentivirus vector.

### Viral Particles

In one aspect, there is provided a recombinant viral particle comprising the recombinant nucleic acid as defined herein. In one embodiment, the recombinant viral particle is a recombinant lentiviral particle.

### Cells

In one aspect, there is provided a cell comprising the recombinant nucleic acid molecule as defined herein.

In one aspect, there is provided an engineered cell expressing at the cell surface membrane the CAR as defined herein. In one embodiment, the engineered cell is an immune cell. In one embodiment, the immune cell is a T-lymphocyte or is derived from T-lymphocytes.

### Use & Methods

“CAR-T” cell therapy uses T cells engineered with CARs for cancer therapy. The premise of CAR-T immunotherapy is to modify T cells to recognize disease cells, typically cancer cells, in order to more effectively target and destroy them. Generally, T are genetically altered to express a CAR, and these cells are infused into a patient to attack their tumors. CAR-T cells can be either derived from T cells in a patient's own blood (autologous) or derived from the T cells of another healthy donor (allogeneic).

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein. In one embodiment, the T-cell is from a donor. In one embodiment, the T-cell is from a patient.

In one aspect, there is providing a method of preparing cells for CAR-T comprising introducing into a T-cell the nucleic acid or vector as described herein. In one embodiment, the T-cell is from a donor. In one embodiment, the T-cell is from a patient.

In one aspect, there is provided a use of the CAR or of the engineered cell as described herein for treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one embodiment, the method further comprises an initial step of obtaining cells from a patient or donor and introducing the recombinant nucleic acid molecule or vector encoding the CAR, as described herein.

In one embodiment, the method further comprises an initial step of obtaining cells from a patient or donor and contacting the cells with the viral particle, as described herein.

In one aspect, there is provided a use of the CAR or of the engineered cell as described herein for preparation of a medicament treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL). In one embodiment, the cancer is a leukemia, lymphoma, multiple myeloma, lung cancer, pancreatic cancer, gastric cancer, colon cancer, or brain cancer.

In one aspect, there is provided the CAR or the engineered cell as described herein for use in treatment of a cancer or an auto-immune disease. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL). In one embodiment, the cancer is a leukemia, lymphoma, multiple myeloma, lung cancer, pancreatic cancer, gastric cancer, colon cancer, or brain cancer.

In one aspect there is provided a method of treating a cancer or an auto-immune disease in a subject, comprising administering to the subject the engineered cell as defined herein. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL). In one embodiment, the cancer is a leukemia, lymphoma, multiple myeloma, lung cancer, pancreatic cancer, gastric cancer, colon cancer, or brain cancer.

### EXAMPLES

### Introduction to the Examples

Anti-CD3 antibodies have been used in a variety of therapeutic modalities. However, many of the current anti-CD3 mAbs are not cross-reactive with cynomolgus or other non-human primate species, which severely limits relevant preclinical toxicity studies and complicates the development pathway for these therapeutics.

Herein is described a collection 16 anti-CD3 monoclonal antibodies (mAbs) that can bind human T-cells (from PBMCs) and Cynomolgus T-cells (Cyno cross-reactive) with similar affinities, a characteristic important to enable risk mitigation associated with anti-CD3 cytokine release syndrome. Importantly, the mAbs can also activate human T-cells when immobilized through plate binding.

A subset of single chain variable fragments (scFv) derived from these mAbs were shown to have similar or greater T cell stimulating activity in bispecific format to blinatumomab (CD19:CD3 bispecific; BsAb) or other tumour targets (NRC developed EGFRvII-specific mAbs:CD3).

Results also indicate that some new mAbs could also mediate internalization as assessed by surrogate ADC testing. The use of CD3-ADC could present a novel treatment for acute or chronic T-cell mediated autoinflammatory diseases. These type of mAbs could be potentially used to improve specificity of delivery of viral vector particles or other nanoparticles to T cells for in vitro or in vivo genetic modification of T cells.

### Example 1: Generation of CD3 Specific Monoclonal Antibodies

### Immunizations

Four six-week old female SJL mice (The Jackson Laboratory, Bar Harbor, ME) were bled (pre-immune serum) and injected 2-3 times IV with at least 6 weeks intervals with a complex mixture of DNA sequences encoding a number of immunogens from CD3 complex. Blood was collected in microvette CB 300Z (Sarstedt, Montreal, QC) and serum was stored at −20° C. until further use.

### Flow Cytometry Analysis (Serum Titer Determination).

Pre- and post-immune sera titer of immunized animals were assessed by flow cytometry on Jurkat T-cell line (Fujisaki Cell Center, Japan). Cells were grown in RPMI1640 medium containing 10% FBS. After centrifugation, cells were resuspended in complete medium at a cell density of 2×10⁶ cells/mL. Unless otherwise stated, all incubations were performed at 4° C. Fifty μL/well of cells were distributed in a polypropylene v-bottom 96 well plate and equal volume of serum dilutions were added and incubated for 2 hours. Cells were washed twice by centrifugation and further incubated with a FITC labeled F(ab′)2 goat anti-mouse antibody (Fc specific, #115-096-071, Jackson Immunoresearch, Cedarlane, Burlington, ON) for an hour. Cells were washed and resuspended in medium containing propidium iodide to exclude dead cells from analysis. Samples were filtered through a 60 μm nylon mesh filter plate (Millipore, Ireland) to remove cell aggregates. Flow cytometry analyses were performed on 2,000 viable single-cells events gated on forward scattering, side scattering parameters and propidium iodide dye exclusion using a BD-LSRFortessa flow cytometer (Becton-Dickinson Biosciences, CA, USA) and a standard filter set using BD FACSDiva™ acquisition software, according to manufacturer's instructions.

Cells were stained with either negative control mouse IgG or positive control OKT3 mAb (company). Specific binding was reflected by the increase in the mean fluorescent intensity of antibody binding to Jurkat T-cells compared to pre-immune serum.

All pre-immune bleeds were negative and post-immune bleeds were positive on Jurkat T-cell line, with titers up to 1/3200.

### Generation of Hybridomas

A total of 8 fusion experiments were performed, with 2 fusions successful. After 3 to 9 months for F332 and F282 respectively, an i.v. booster injection of antigen was done 4 days prior to fusion experiments.

Fusion of the harvested spleen cells. All manipulations were done under sterile conditions. Spleen cells and NS0 myeloma cells were harvested and washed separately in IMDM. Cells were further washed in Isoosmolar buffer (Eppendorf cat #4308070.536), then in Hypoosmolar buffer (Eppendorf cat #4308070.528) for F282 or Cytofusion Medium C (BTX cat #47-0001) for F332. Myeloma and lymphocytes were counted in RBC lysing buffer (Sigma, Cat #7757-100ML), mixed together at a 1:1 ratio and fused using an ECM 2001 Cell Fusion System (BTX, Harvard Bioscience Inc.) following manufacturer's instructions.

Following fusion, cells were suspended at a concentration of 2×105 input myeloma cells per ml in HAT selection medium (IMDM containing 20% heat inactivated FBS, penicillin-streptomycin (Sigma Cat #P7539), 1 ng/ml mouse IL-6 (Biolegend Cat #575706), HAT media supplement (Sigma Cat #H0262) and L-glutamine (Hy-Clone Cat #SH30034.01) and incubated at 37° C., 5% CO₂. The next day, hybridoma cells were washed and suspended at a concentration of 2-3×10⁵ input myeloma cells per ml in semi-solid medium D (StemCell Technologies Cat. #03804) supplemented with 5% heat inactivated FBS, 1 ng/ml mouse IL-6 and 10 μg/ml FITC-F(ab′)₂ Goat anti-mouse IgG (Jackson #115-096-071). The cell mixture was plated in Omnitray dish (Nunc cat #242811) and further incubated for 6-7 days at 37° C., 5% CO₂. Fluorescent secretor clones were then transferred using a mammalian cell clone picker (ClonepixFL, Molecular Devices) into sterile 96-w plates (Costar #3595) containing 200 μl of IMDM supplemented with 20% heat inactivated FBS, penicillin-streptomycin, 1 ng/ml mouse IL-6, HT media supplement (Sigma Cat #H0137) and L-glutamine and incubated for 2-3 days at 37° C., 5% CO₂.

Cell-ELISA to detect specific binders. Unless otherwise stated, all incubations are performed at 4° C. On the day of the assay, CHO3E7-CD3 or control CHO cells are thawed, centrifuged, resuspend in medium:Starting block (1:1) at 2×10⁶ cells/mL and seeded at 50 uL/well in 96-well filter plates. Equal volumes of supernatant are added to cells, mixed and incubated for 2 hours at 4° C. Plates are washed 3 times using vacuum filtration. An HRP-conjugated anti-mouse IgG Fc gamma specific secondary antibody (Jackson Immunoresearch cat #115-036-071) is added to the wells and further incubated for 1 hour at 4° C. Plates are washed 5 times by vacuum filtration followed by the addition of TMB substrate (Pierce cat #34021) at room temperature. The reaction is stopped by adding 1 M sulfuric acid and supernatant is transferred into a clear 96-w plate. Absorbance (450 nm) is read on a Spectramax 340PC plate reader with path-check correction.

Hybridoma supernatants were screened by flow cytometry on Jurkat T-cell line as described above (serum titer determination), using 50 μL of hybridoma supernatant per well instead of serum dilution. Positive hybridoma supernatants were counterscreened on Jurkat derived J.RT3-T3.5 T-cell line (ATCC) that lacks the beta chain and is negative for CD3 expression.

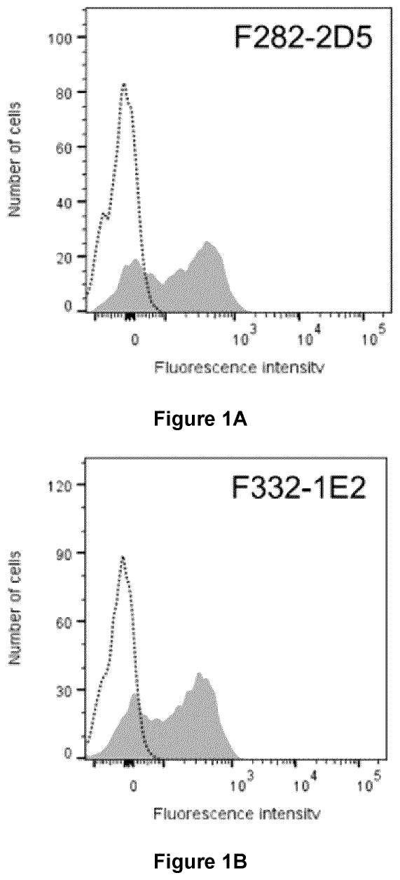

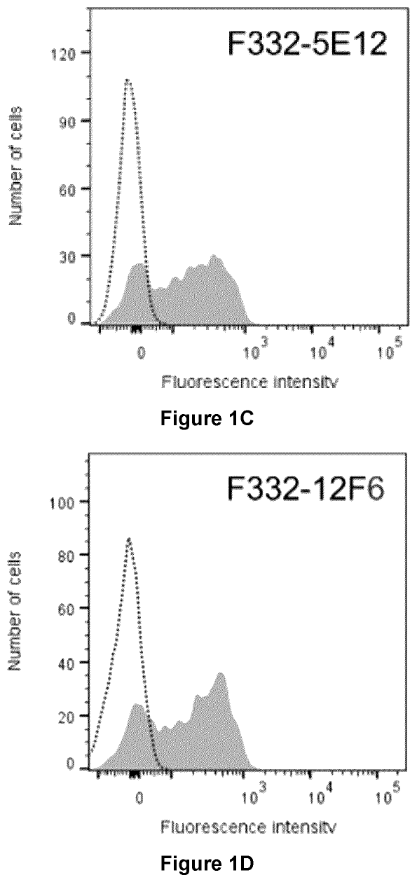

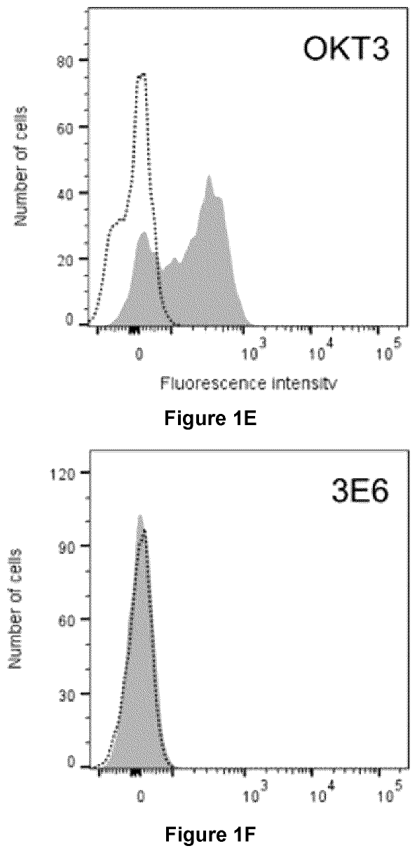

FIGS. 1A-1F depict histograms obtained using flow cytometry on supernatants of selected hybridomas on CD3 positive Jurkat (shaded histogram) or CD3 negative J.RT3-T3.5 (empty histogram) cell lines.

Cells were stained with either negative control mouse IgG (Jackson Immunoresearch cat #115-000-003) or an anti-GFP 3E6 mAb supernatant or tested hybridoma supernatant. Specific binding was reflected by the increase in the mean fluorescent intensity of antibody binding to Jurkat T-cells (FIGS. 1A to 1F, grey histograms) compared to the Jurkat CD3 negative cell line J.RT3-T3.5 (FIGS. 1A to 1F, open histograms). AF488 labeled positive control mAb OKT3 was also analyzed.

A total of 38 clones were found positive and specific to Jurkat T-cell line: one clone from fusion F282 and 37 clones from fusion F332. Examples of flow cytometry specific binding are shown in FIGS. 1A to 1F with clones F282-2D5 (FIG. 1A), F332-1E2 (FIG. 1B), F332-5E12 (FIG. 1C), F332-12F6 (FIG. 1D) and OKT-3 positive control (FIG. 1E). Negative controls mouse IgG or 3E6 (anti-GFP) are shown in FIG. 1F and FIG. 1G respectively.

Table 1 depicts the mean fluorescence intensity of flow cytometry analysis of selected anti-CD3 mAbs supernatant on Jurkat and J.RT3-T3.5 T-cell lines.

TABLE 1
Mean fluorescence intensity of flow cytometry analysis of selected
anti-CD3 mAbs supernatant on Jurkat and J.RT3-T3.5 T-cell lines.

| | Jurkat | |
|---|---|---|
| Clone | (MFI) | J.RT3-T3.5 (MFI) |
| F282-2D5 | 349 | 26 |
| F332-1C9 (1A10, 5H8, 8B11) | 277 | 20 |
| F332-1E2 (8C5) | 319 | 21 |
| F332-3E9 (12G4) | 297 | 23 |
| F332-4D6 | 398 | 20 |
| F332-5D4 (5F8) | 326 | 18 |
| F332-5F2 (6G8, 13A3) | 292 | 23 |
| F332-6D7 (7G4) | 247 | 27 |
| F332-7B7 | 302 | 18 |
| F332-7C5 (5E12, 10A8) | 334 | 19 |
| F332-7C9 (5C7, 7D10) | 285 | 20 |
| F332-7E3 | 354 | 25 |
| F332-8F7 | 312 | 22 |
| F332-10B6 | 143 | 21 |
| F332-12F6 | 369 | 22 |
| F332-13B4 | 373 | 25 |
| OKT-3 AF488 | 265 | 28 |
| SK7 AF488 | 311 | 19 |
| UCHT1 AF488 | 293 | 16 |
| 3E6-2 (anti-GFP) | 22 | 19 |
| Mouse IgG | 34 | 31 |

### Example 2: Evaluation of Apparent Affinity by Flow Cytometry

mAbs Purification.

Selected mAbs supernatant were produced and purified on Protein G or Protein A columns and desalted on HiPrep or Zeba Spin or Centripure P2 desalting columns pre-equilibrated in PBS and filter sterilized through 0.22 μM membrane (Millipore). Purified mAbs were concentrated using Vivaspin turbo concentrators (30 kDa) and SEC purified on Superdex 200 to remove aggregates. The final concentration of the antibody solutions was determined using a Nano-drop 2000 (ThermoScientific) or a Cytation 5 micro-volume Take3 plate (Biotek), using IgG as sample type.

Purified anti-CD3 monoclonal antibodies were assessed for their binding activity by flow cytometry in a dose-dependent binding curve using Jurkat T-cell line. A recombinant mouse IgG2b OKT-3 produced in-house was also included in the study.

Unless otherwise stated, all media are kept are 4° C. and all incubations are performed on wet ice. Exponentially growing Jurkat cells were centrifuged and resuspended in complete medium at a cell density of 2×10⁶ cells/mL. Fifty μL/well of cells were distributed in a polypropylene v-bottom 96 well plate and serial 1/3 dilutions of purified mAbs starting at 300 nM were added and incubated for 2 hours. Cells were washed twice by centrifugation and further incubated with a FITC labeled F(ab′)2 goat anti-mouse antibody (Fc specific, #115-096-071, Jackson Immunoresearch, Cedarlane, Burlington, ON) for an hour. Cells were washed and re-suspended in medium containing Propidium iodide to exclude dead cells from analysis. Samples were filtered through a 60 μm nylon mesh filter plate (Millipore, Ireland) to remove cell aggregates. Flow cytometry analyses were performed on 2,000 viable single-cells events gated on forward scattering, side scattering parameters and propidium iodide dye exclusion using a BD-LSRFortessa flow cytometer (Becton-Dickinson Biosciences, CA, USA) and a standard filter set using BD FACSDiva™ acquisition software, according to manufacturer's instructions.

Commercially available AF488-labeled anti-CD3 mAbs OKT-3, SK7, UCHT1 (Biolegend, Cedarlane, Burlington, ON) and SP34.2 (BD Bioscience, San Jose, CA, USA) were assessed in the same manner, except that the secondary antibody incubation step was omitted. The recombinant mouse IgG2b OKT-3 produced in-house was conjugated to AlexaFluor 488 fluorescent dye (Thermofisher, Burlington, ON, Canada) using a NHS Ester derivative, according to manufacturer's instructions and was also included.

Specific detection of antibody binding was calculated as the mean fluorescent intensity of binding to each primary antibody after background level subtraction of the mean fluorescent intensity of binding of mouse IgG control. The data were analyzed with GraphPad Prism v 9.0 software using one-site specific binding with Hill slope non-linear regression curve fit model to determine Bmax (maximum specific binding) and Kdapp (concentration needed to achieve a half-maximum binding at equilibrium) for each mAb tested.

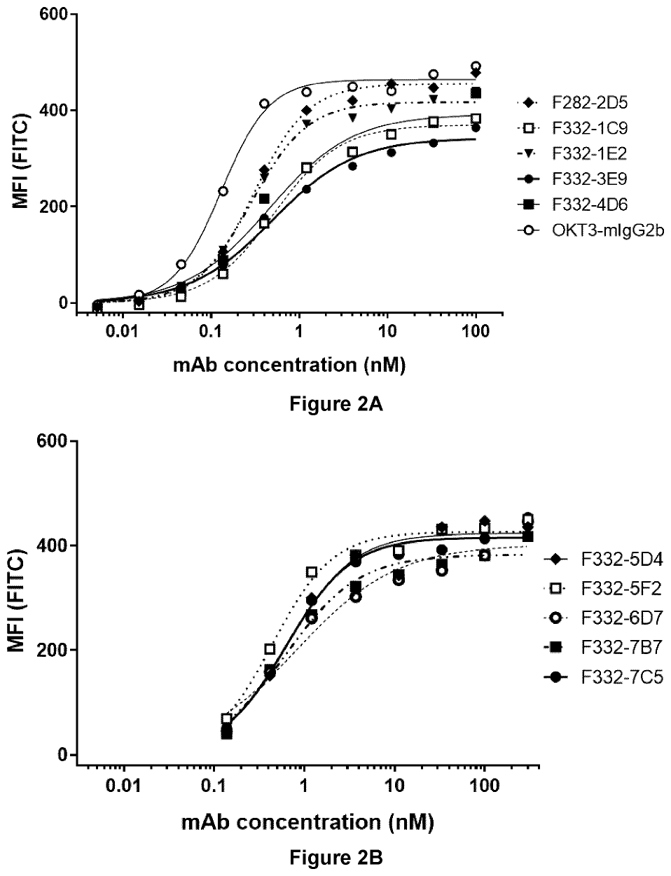

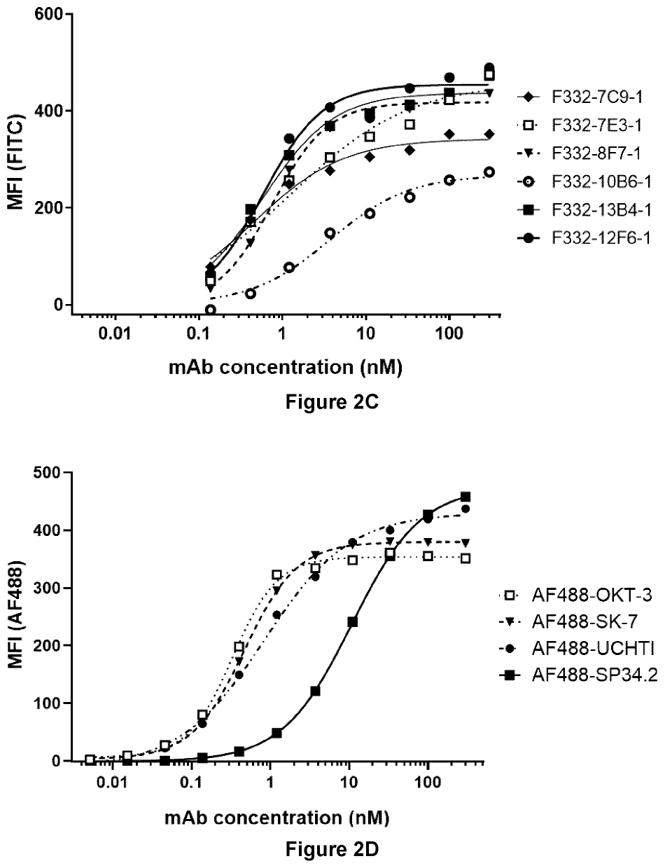

FIGS. 2A to 2D depict dose-response binding curves obtained from flow cytometry data of purified monoclonal antibodies on Jurkat human T cell line expressing CD3. All anti-CD3 mAbs showed strong and comparable binding to CD3 positive Jurkat cells (FIGS. 2A-2C). Except for F332-7E3 (1.3 nM), −10B6 (3.7 nM) and SP34.2 Cynomolgus cross-reactive (10.8 nM) antibodies, all monoclonal antibodies including directly labeled commercial and in-house mAbs bind with sub-nM affinity to Jurkat cells in flow cytometry analysis (Table 3a).

Selected hybridoma were recloned by limiting dilution to ensure their monoclonality. Their respective subclass was determined using IsoStrip™ Mouse Monoclonal Antibody Isotyping Kit.

As shown in FIGS. 2A to 2D and Table 2, all mAbs bind to Jurkat with apparent KD of 0.3-3.7 nM.

TABLE 2
Apparent KD and subclass of anti-CD3 mAbs on various Cd3
expressing target [symbol] determined by flow cytometry analysis

| | | K_{D}app (nM) | | | |
|---|---|---|---|---|---|
| | | | | HSC-F | |
| | | | | (NHP | |
| | | | Cyno | T-cell | Human |
| Clone | Isotype | Jurkat | PBMCs | line) | T ce [symbol] |
| F282-2D5 | IgG2a, κ | 0.3 | 0.11 | ND | 0.47 |
| F332-1C9 | IgG2a, κ | 0.5 | 0.25 | ND | 0.94 |
| (1A10, 5H8, | | | (0.26) | | |
| 8B11) | | | | | |
| F332-1E2 | IgG2a, κ | 0.3 | 0.28 | 0.39 | 0.40 |
| (8C5) | | | | | |
| F332-3E9 | IgG2a, κ | 0.5 | 0.21 | ND | 1.0 |
| (12G4) | | | | | |
| F332-4D6 | IgG2a, κ | 0.5 | 0.15 | ND | 0.53 |
| F332-5D4 | IgG2b, κ | 0.7 | | 0.64 | 0.61 |
| (5F8) | | | | | |
| F332-5F2 | IgG2b, κ | 0.4 | | 0.4 | 0.37 |
| (6G8, 13A3) | | | | | |
| F332-6D7 | IgG2a, κ | 0.8 | | 0.27 | 0.46 |
| (7G4) | | | | | |
| F332-7B7 | IgG2a, κ | 0.6 | | 0.28 | 0.23 |
| F332-7C5 | IgG2a, κ | 0.6 | | 0.37 | 0.41 |
| (5E12, 10A8) | | | | | |
| F332-7C9 | IgG2a, κ | 0.5 | | 0.36 | 0.16 |
| (5C7, 7D10) | | | | | |
| F332-7E3 | IgG2a, κ | 1.3 | | 0.31 | 0.22 |
| F332-8F7 | IgG2a, κ | 0.8 | | 0.97 | 0.26 |
| F332-10B6 | IgG2a, κ | 3.7 | | 2.0 | 30.1 |
| F332-12F6 | IgG2a, κ | 0.6 | | 0.61 | 0.20 |
| F332-13B4 | IgG2a, κ | 0.6 | | 0.64 | 0.05 |
| OKT-3 | IgG2b, κ | 0.13-0.23 | | ndb | 0.03 |
| mIgG2b | | | | | |
| AF488-SP34.2 | IgG1, λ | 10.8 | | ND | ND |
| SP34.2 | IgG1, λ | ND | | 1.29 | 1.77 |
| 3E6-2 | IgG2a, κ | ndb | | ND | Y |
| (anti-GFP) | | | | | |

[symbol]

indicates data missing or illegible when filed

### Example 3: Evaluation of Binding on Human PBMC

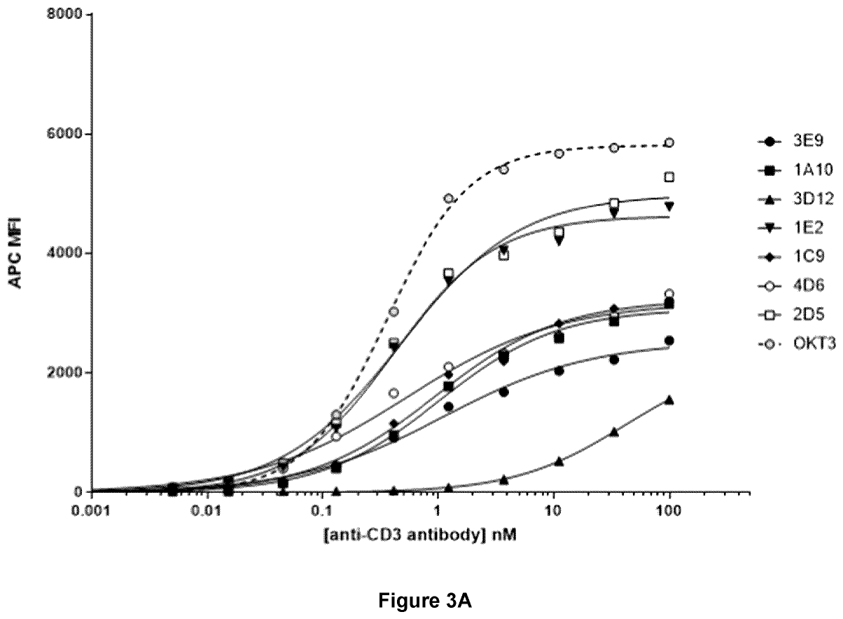

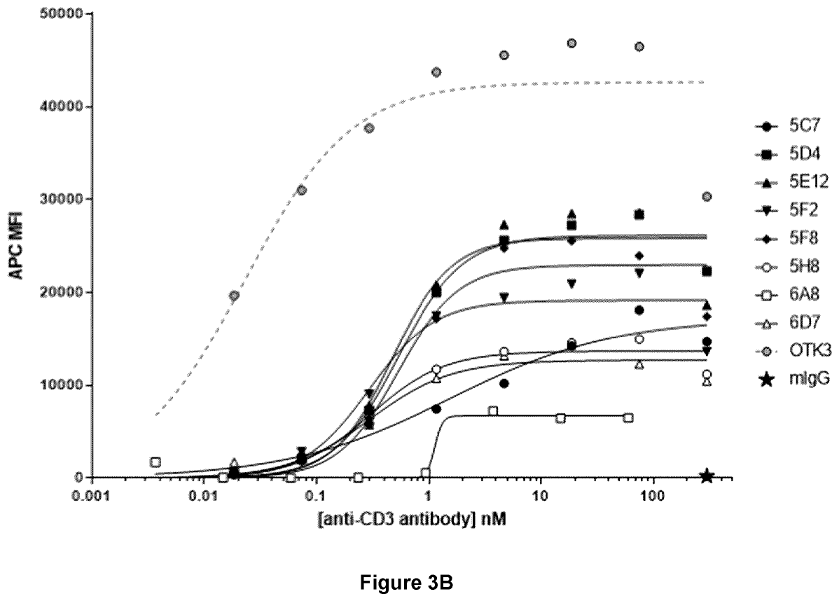

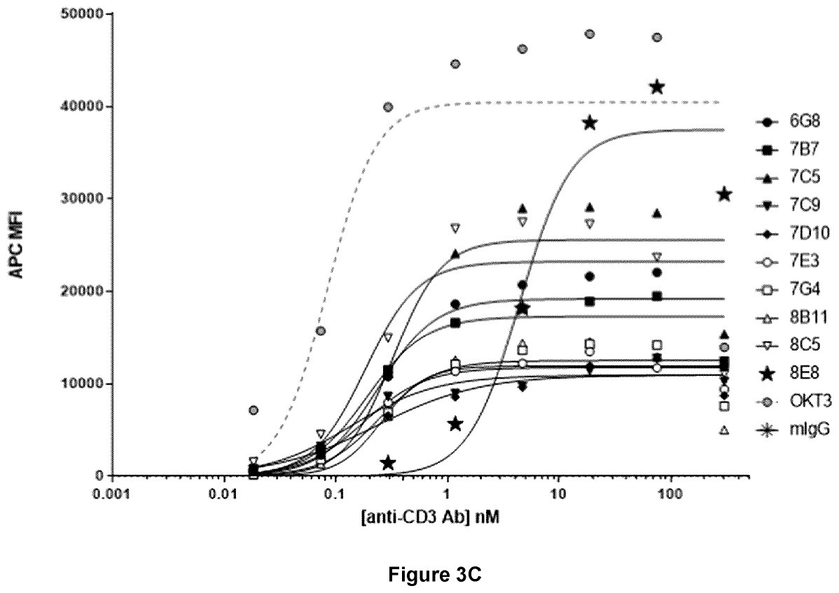

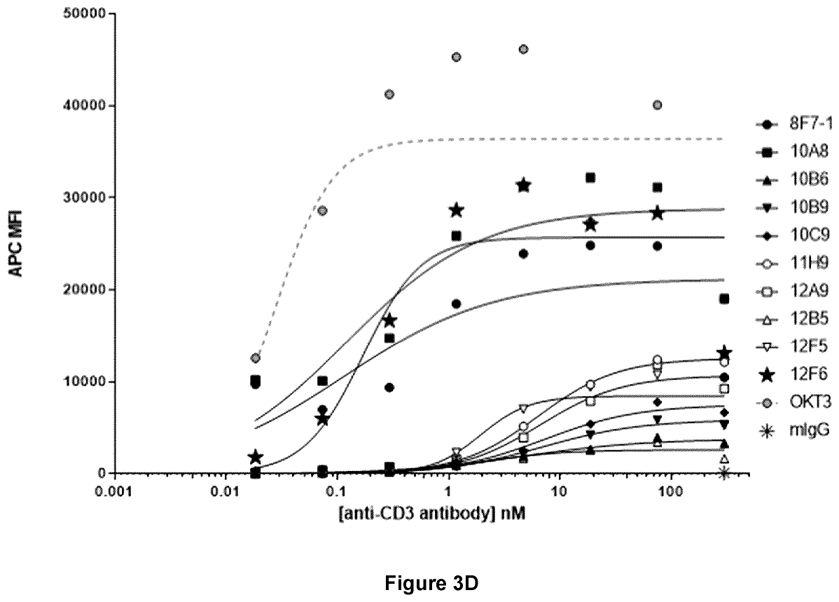

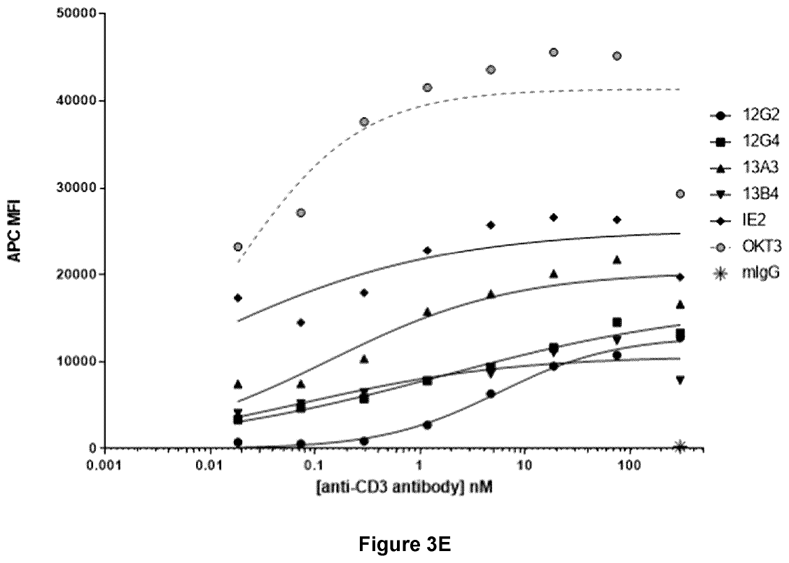

The binding characteristics of the purified anti-CD3 mAbs were assessed in titration studies on purified human primary T cells from different donor samples. Fresh ex vivo peripheral blood mononuclear cells (PBMCs) were obtained via density centrifugation over Ficoll-Paque using standard procedures and untouched T cell fractions obtained from PBMCs by negative pan-T cell selection and magnetic cell sorting. Starting from 300 nM concentrations of the anti-CD3 mAbs, eight point binding curves were generated from the resultant mean fluorescence intensity (MFI) values obtained through detection of labelled secondary antibody to bound CD3 in flow cytometry experiments (FIGS. 3A and 3B). Both commercial mouse anti-human CD3 mAb (OKT-3; Biolegend) and an in house generated IgG1 version of OKT-3 were employed as positive controls together with a non-specific mouse IgG serving as a negative binding control. Following curve fit analysis using one-site specific binding with Hill slope, the KDapp (concentration needed to achieve a half-maximum binding at equilibrium) for each mAb was calculated (Table 2).

All anti-CD3 mAbs possessed KDapp affinities in the range of 100 pM to 1 nM with the exception of F332-10B6 with >30 nM. This latter result may reflect mAb instability over time relative to KDapp determinations on Jurkat T cells. In general, mAb binding to primary human T cells was somewhat stronger relative to the Jurkat T cell line (reflected by lower KDapp) with up to 7 fold concentration decreases for half maximal binding to primary T cells. The anti-CD3 mAbs showed variable maximum specific binding (Bₘₐₓ) (data not shown) on human primary T cells indicating a degree of clonal variation.

Eight point binding titrations were performed with the anti-CD3 mAbs starting at either 300 or 100 nM on freshly purified human T cells obtained through negative selection from PBMC fractions. Briefly, 1×10⁵ human T cells (50 μL of staining buffer (SB; PBS/1% BSA/0.01% NaN3) were incubated with graded dilutions of the anti-CD3 mAbs in SB (50 L) for 2 hours at 4° C. in 96 w U-bottom plates, followed by plate washing with SB (150 L). Cell pellets were resuspended with 25 μL of a 0.25 mg/mL solution of the goat (Fab′)₂ anti-mouse IgG (H+L)− APC secondary antibody in SB, incubated at 4° C. (30 min.), washed 1× with SB, and resuspended for flow analysis. Recombinant anti-human OKT-3 IgG2b (in house produced) and non-specific mIgG were employed similarly as positive and negative controls respectively. 10K live events were acquired on a BD LSRFortessa flow analyzer and mean fluorescence intensities (MFI) calculated after subtraction of secondary antibody alone values. Using GraphPad Prism software, the data were analyzed using a one site specific binding with Hill slope non-linear regression model to calculate KDapp and B_{Max} values (See Table 2).

### Example 4: Evaluation of Binding on Fresh Cynomolgus PBMC or HSC-F Cynomolgus T-Cell Line

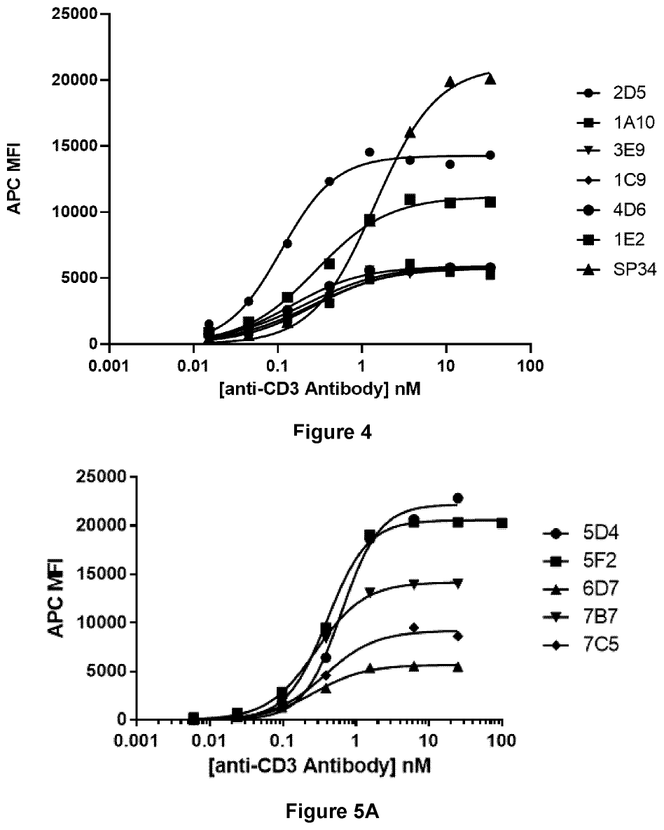

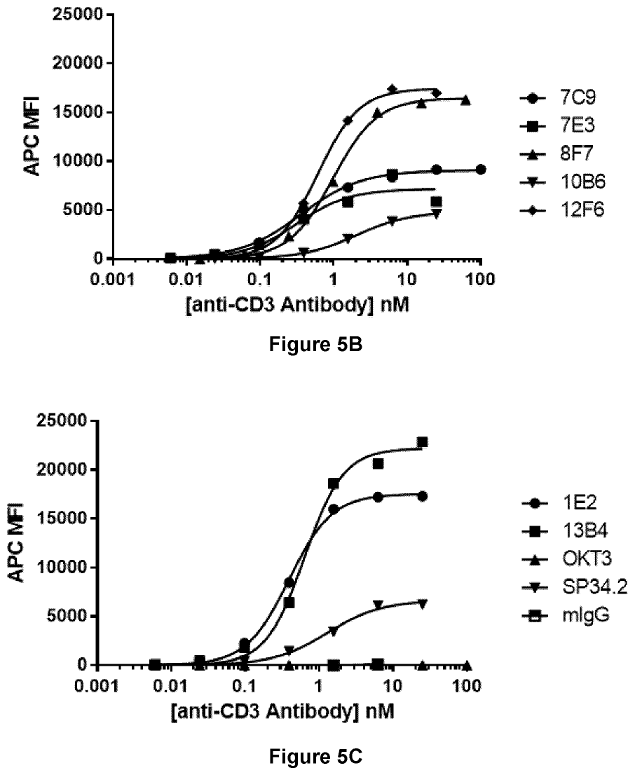

It is of significant interest for product development to ascertain anti-human CD3 cross-reactivity in non-human primate (NHP) tissues. To characterize potential cross-reactivity of the anti-human CD3 mAbs to NHP T cells, dose response flow cytometry titration studies were performed on both freshly obtained Cynomolgus PBMCs (Comparative Medicine and Animal Resources Centre—McGill University) or the Cynomolgus derived T cell line, HSC-F (Non-Human Primate Reagent Resource—NIH). Binding studies on fresh Cynomolgus PBMCs was performed on a subset of anti-CD3 mAbs (FIG. 4) whereas the Herpesvirus saimiri immortalized Cynomolgus T cell line HSC-F was employed for further evaluations (FIGS. 5A to 5C). KDapp values were calculated using a non-linear regression model of one-site binding with Hill slope (GraphPad Prism) and tabulated (Table 3b). Similar KDapp values for anti-CD3 mAb 1E2 obtained from binding to fresh Cynomolgus PBMCs (KDapp=0.28 nM) or the HSC-F T cell line (KDapp=0.39 nM) indicated the HSC-F cell line was a suitable surrogate for ex vivo NHP T cells. With the exception of mAbs 10B6 (2.0 nM) and the commercial SP34.2 (1.29 nM), all anti-CD3 mAbs possessed sub-nM affinity to NHP T cells paralleling the KDapp values obtained with binding to human T cells (Table 3).

Cynomolgus PBMCs were obtained from fresh ex vivo peripheral blood via density centrifugation and cultured overnight at 37° C./5% CO₂ in the presence of human recombinant IL2 (20 IU/mL; Novartis). Starting from 100 nM, graded doses of the anti-CD3 mAbs were incubated with 100K NHP PBMCs for 0.5 hours at 4° C. in flow staining buffer (SB; PBS/1% BSA/0.01% NaN3) in 96 w V-bottom plates. Following plate washing with SB (150 μL), cell pellets were resuspended in a 5 □g/mL solution of goat (Fab′)₂ anti-mouse IgG (H+L)− APC secondary antibody in SB, incubated at 4° C. (30 min.), washed 1× with SB, and resuspended for flow analysis. The commercial NHP cross-reactive SP34.2 anti-CD3 mAb (BD Biosciences), anti-human OKT-3 IgG2a (Biolegend—data not shown) and non-specific mIgG were employed as controls. 20K live events were acquired on a BD LSRFortessa flow analyzer and mean fluorescence intensities (MFI) calculated after subtraction of secondary antibody alone values. Using GraphPad Prism software, the data were analyzed using a one site specific binding with Hill slope non-linear regression model to calculate KDapp and B_{Max} values (See Table 2).

HSC-F T cells obtained from the Non-Human Primate Reagent Resource (NIH) were maintained in culture in RPMI media containing 20% FBS and 40 IU/mL recombinant human IL2. 100K HSC-F cells in logarithmic cell growth were treated with graded doses of the anti-CD3 mAbs starting at 100 nM in staining buffer (SB; PBS/1% BSA/0.01% NaN3) for 2 hours at 4° C. in 96 w V-bottom plates. Following plate washing with SB, the cell pellets were resuspended with 25 μL of goat (Fab′)₂ anti-mouse IgG (H+L)− APC secondary antibody (final 5 μg/mL), incubated for 30 minutes at 4° C., followed by flow cytometry analysis. The commercial NHP cross-reactive SP34.2 anti-CD3 mAb (BD Biosciences), recombinant anti-human OKT-3 IgG2b (in house produced) and non-specific mIgG were employed as controls. 20K live events were acquired on a BD LSRFortessa flow analyzer and mean fluorescence intensities (MFI) calculated after subtraction of secondary antibody alone values. Using GraphPad Prism software, the data were analyzed using a one site specific binding with Hill slope non-linear regression model to calculate KDapp and B_{Max} values (See Table 2).

### Example 5: Epitope Binning by Flow Cytometry Competition

In order to evaluate whether anti-CD3 monoclonal antibodies bind to the same epitope region, flow cytometry competition experiments on Jurkat cells were performed.

To that effect, a fixed concentration of the indicated AF488-labeled mAb were incubated with 100 nM of unlabeled monoclonal antibodies to assess if their binding capacity was impaired in presence of unlabeled mAbs. The anti-CD3 tested were AF488-OKT3 mIgG2b used at 1.5 nM, AF488-SP34.2 (BD Pharmingen #557705) used at 20 nM, AF488-UCHT1 (Biolegend #300415) used at 5 nM and AF488-SK7 (Biolegend #344810) used at 1.5 nM. The AF488-OKT3 mIgG2b was labeled in-house using AlexaFluor488 protein labelling kit (Invitrogen #A10235) following manufacturer's instruction. The free dye removal step was done using Zeba™ Spin Desalting Columns, 7K MWCO (Thermo Scientific #89890). The final product was analyzed by UPLC-SEC to ensure the integrity of the labeled mAb.

Data in Table 3A represents the percentage of residual fluorescence in presence of 100 nM of unlabeled mAb compared to the fluorescence in absence of competitor. From the results shown in Table 3A (and summarized in Table 3B), it can be seen that the selected anti-CD3 monoclonal antibodies fall into 1 major epitope regions characterized by binding of 4 prototypic antibodies; all antibodies did show similar competition profile as the NHP cross-reactive SP34.2, i.e. they did strongly compete with AF488-SP34.2, did not compete with labeled OKT-3, and showed partial competition with labeled SK7 and labeled UCHT1.

TABLE 3A
Competition Assay by flow cytometry. Percentage of residual
fluorescence of labeled anti-CD3 commercial mAbs in
presence of 100 nM of unlabeled tested anti-CD3 mAbs.

| | AF488 Labeled mAbs | | | |
|---|---|---|---|---|
| mAbs | OKT-3 | SP34.2 | UCHT1 | SK7 |
| F282-2D5 | 78 | 7 | 64 | 42 |
| F332-1C9 | 89 | 12 | 76 | 45 |
| (1A10, 5H8, 8B11) | | | | |
| F332-1E2 (8C5) | 77 | 6 | 69 | 42 |
| F332-3E9 (12G4) | 79 | 11 | 76 | 45 |
| F332-4D6 | 78 | 9 | 76 | 44 |
| F332-5D4 (5F8) | 93 | 10 | 73 | 49 |
| F332-5F2 | 90 | 9 | 74 | 49 |
| (6G8, 13A3) | | | | |
| F332-6D7 (7G4) | 89 | 10 | 73 | 44 |
| F332-7B7 | 86 | 9 | 79 | 53 |
| F332-7C5 | 79 | 7 | 64 | 39 |
| (5E12, 10A8) | | | | |
| F332-7C9 | 82 | 9 | 68 | 42 |
| (5C7, 7D10) | | | | |
| F332-7E3 | 79 | 12 | 67 | 45 |
| F332-8F7 | 87 | 10 | 59 | 39 |
| F332-10B6 | 94 | 32 | 80 | 56 |
| F332-12F6 | 82 | 7 | 63 | 42 |
| F332-13B4 | 79 | 8 | 72 | 43 |
| OKT-3 mIgG2b | 8 | 26 | 11 | 6 |
| 3E6-2 (anti-GFP) | 104 | 101 | 98 | 95 |
| SK7 | ND | 58 | 14 | 9 |
| UCHT1 | ND | 54 | 8 | 7 |
| SP34.2 | ND | 10 | 49 | 29 |

“ND” = not determined.

TABLE 3B
Summary of Table 3A

| | Labeled mAbs | | | |
|---|---|---|---|---|
| mAbs | OKT-3 | SP34.2 | UCHT1 | SK7 |
| F282-2D5 | NC | C | PC | PC |
| F332-1C9 | NC | C | NC | PC |
| (1A10, 5H8, 8B11) | | | | |
| F332-1E2 (8C5) | NC | C | PC | PC |
| F332-3E9 (12G4) | NC | C | NC | PC |
| F332-4D6 | NC | C | NC | PC |
| F332-5D4 (5F8) | NC | C | PC | PC |
| F332-5F2 | NC | C | PC | PC |
| (6G8, 13A3) | | | | |
| F332-6D7 (7G4) | NC | C | PC | PC |
| F332-7B7 | NC | C | NC | PC |
| F332-7C5 | NC | C | PC | PC |
| (5E12, 10A8) | | | | |
| F332-7C9 | NC | C | PC | PC |
| (5C7, 7D10) | | | | |
| F332-7E3 | NC | C | PC | PC |
| F332-8F7 | NC | C | PC | PC |
| F332-10B6 | NC | PC | NC | PC |
| F332-12F6 | NC | C | PC | PC |
| F332-13B4 | NC | C | PC | PC |
| OKT-3 mIgG2b | SC/C | PC | C | C |
| 3E6-2 (anti-GFP) | NC | NC | NC | NC |
| SK7 | ND | PC | C | SC/C |
| UCHT1 | ND | PC | SC/C | C |
| SP34.2 | ND | SC/C | PC | PC |

“NC” = no competition (>75%); “SC” = self-competition; “C” = competition (<20%); “PC” = partial competition (21-75%); “ND” = not determined.

### Example 6: Evaluation of T-Cell Activation

CD69 expression is rapidly induced in T-cells following CD3/TCR engagement. In order to evaluate T-cell activation potential of anti-CD3 mAbs, a Jurkat T-cell line was engineered to express dTomato under CD69 promoter (Bloemberg et al. 2020). Thus, the induction of dTomato fluorescent protein expression can be used as a surrogate marker to determine the potential for T-cell activation.

Exponentially growing Jurkat-CD69-dTomato cells were centrifuged and resuspended in at a cell density of 10⁶ cells/mL in complete medium containing anti-CD28 at at 2 μg/mL and anti-mouse IgG at 5 μg/mL. Fifteen μL/well of 1/2 serial dilutions of purified mAbs starting at 2-4 μg/mL were distributed in a Corning® 384-Well Flat Clear Bottom Black Polystyrene TC-treated Microplates. Corning®#3764 and 15 μL/well of cell suspension were added and incubated at 37° C., 5% C02 and read at 3 h intervals for 48 hrs in the Incucyte using Phase and Red channels, 10× objective. Data were recorded as red intensity per cell area and were analyzed with GraphPad Prism v 9.0 software using log(inhibitor) vs. response—Variable slope (four parameters) to determine EC50 and Span (distance between Top and Bottom plateaus) at 24-hr timepoint. Data were normalized to OKT-3 positive control, where 100% was defined as the fluorescence intensity per cell area of the highest dose of OKT3 and 0% was defined as the fluorescence intensity per cell area of the non-treated controls.

Table 4 depicts results. All mAbs were able to induce dTomato expression which is under 0069 promoter, with EC50 ranging from 0.1-0.5 μg/ml (0.7-3.3 nM). The percentage of OKT-3 maximal activation range from 60 to 110%. As expected, the negative control 3E6 mAb (anti-GFP) did not induce dTomato expression.

TABLE 4
Evaluation of anti-CD3 mAbs activation potency on
Jurkat dTomato-CD69

| | EC50, μg/mL | % max ref. |
|---|---|---|
| Clone | (n=) | OKT3 |
| F282-2D5 | 0.13 (1) | 98 |
| F332-1C9 (1A10, 5H8, 8B11) | 0.20 ± 0.02 (3) | 94 ± 6.1 |
| F332-1E2 (8C5) | 0.16 ± 0.04 (5) | 104 ± 12.4 |
| F332-3E9 (12G4) | 0.11 ± 0.05 (4) | 84 ± 9.3 |
| F332-4D6 | 0.18 (1) | 87 |
| F332-5D4 (5F8) | 0.27 ± 0.03 (3) | 109 ± 12.5 |
| F332-5F2 (6G8, 13A3) | 0.19 ± 0.03 (5) | 98 ± 9.8 |
| F332-6D7 (7G4) | 0.25 ± 0.02 (2) | 86 ± 4.6 |
| F332-7B7 | 0.25 (1) | 97 |
| F332-7C5 (5E12, 10A8) | 0.23 ± 0.03 (4) | 102 ± 13.4 |
| F332-7C9 (5C7, 7D10) | 0.24 ± 0.01 (3) | 91 ± 5.1 |
| F332-7E3 | 0.24 (1) | 75 |
| F332-8F7 | 0.22 (1) | 80 |
| F332-10B6 | 0.23 ± 0.04 (2) | 60 ± 1.9 |
| F332-12F6 | 0.25 ± 0.01 (2) | 87 ± 5.6 |
| F332-13B4 | 0.47 ± 0.23 (2) | 90 ± 20.4 |
| OKT-3 recomb.(mouse IgG2b) | 0.17 ± 0.02 (4) | 105 ± 3.1 |
| 3E6-2 | Inactive (3) | 0-3 |

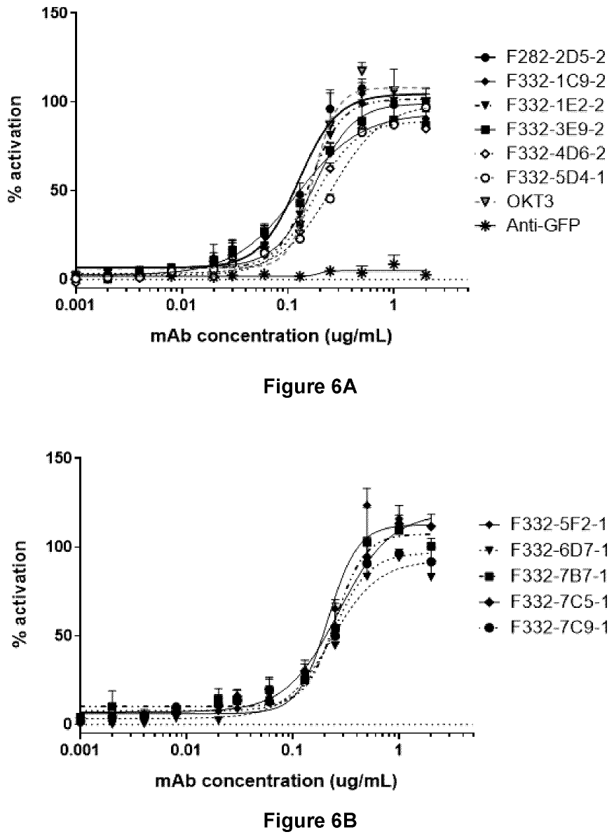

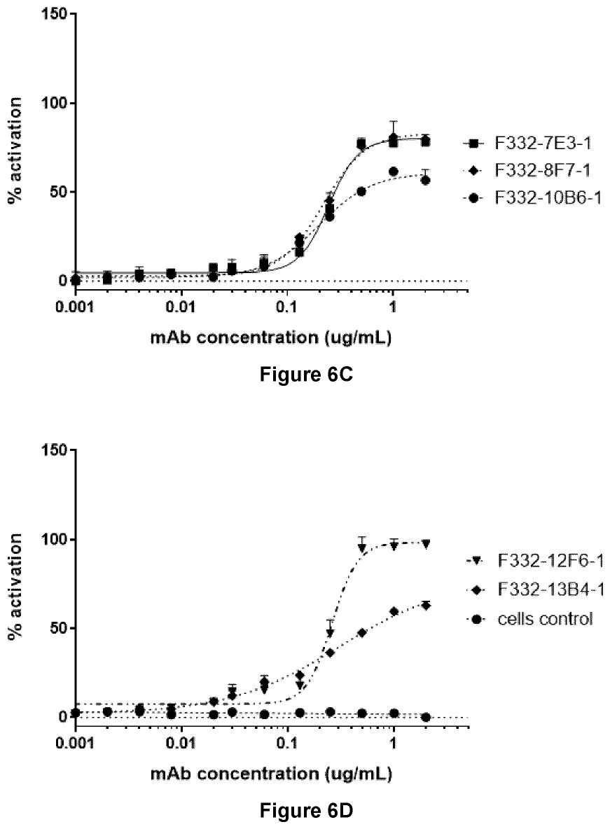

FIGS. 6A to 6D are graphs representing the percentage of reference mAb OKT-3 maximal activation in the presence or absence of anti-CD3 mAbs measured by examining the level of dTomato-reporter gene under 0069 promoter.

### Example 7: Functional Characterization for Antibody Internalization Using an Indirect Surrogate Antibody-Drug Conjugate (ADC) Potential Assay

The purified anti-CD3 monoclonal antibodies were evaluated for their ability to internalize in CD3 expressing Jurkat or CD3 negative J.RT3-T3.5 T-cells using a surrogate ADC growth inhibition assay in which anti-mouse Fc secondary antibodies were coupled to the DM1 maytansine drug through a non-cleavable linker. Once internalized, linker catabolism in the lysosome releases active DM1 drug which destabilizes microtubules and causes growth inhibition.

Generally, cells were passaged 3 times a week and used within 4-6 weeks for all experiments. Cells (Jurkat or J.RT3-T3.5) were seeded at 1,500 cells/25 μL/well in 384-well plates (Corning 3764). Purified mAbs were pre-incubated for 30 min with equimolar amounts (1:1:1) of anti-mouse IgG1 and anti-mouse IgG2a secondary antibodies chemically conjugated with Maytansine (DM1), a tubulin inhibitor that needs to be internalized in order to cause growth inhibition. After pre-incubation, serial 1/4 dilutions of the reaction mixes were prepared and the antibody complexes were then added to the cells at final concentrations of 100 nM to 0.0001 nM. Effects on cell proliferation were measured after 3 days of incubation at 37° C. Incubation with an irrelevant primary antibody (3E6 anti-GFP mAb) was used to assess non-target-directed cytotoxicity. Cell proliferation was determined using CellTiterGlo™ (Promega, Madison), based on quantitation of the ATP present in each well, which signals the presence of metabolically active cells. Signal output was measured on a luminescence plate reader (Envision, Perkin Elmer) set at an integration time of 0.1 sec. Integration time is adjusted to minimize signal saturation at high ATP concentration.

Dose-response curves were generated to measure their potency (IC₅₀) and efficacy (% maximal inhibition) using the log(inhibitor) vs. response—Variable slope (four parameters) model from GraphPad Prism software.

All the tested anti-CD3 antibodies demonstrated good efficacy (from 91-97% maximal growth inhibition) and strong potency (IC50<0.03-0.17 nM) on CD3 expressing Jurkat cells as compared to the non-targeted irrelevant anti-GFP mouse IgG negative control (Table 6). Furthermore, this effect was specific for CD3-expressing cells, since activity seen in the CD3 negative J.RT3-T3.5 cells (IC₅₀ range=9.0-12.0 nM) was not significantly different than that seen for the irrelevant 3E6 IgG control (IC50-10 nM).

Table 5 shows results of the ADC surrogate screening assay of anti-CD3 monoclonal antibodies on CD3 positive (Jurkat) or negative (J.RT3-T3.5) cells. Results are expressed as the percentage of survival relative to that of non-treated cells (incubated with secondary antibody alone) (set at 100%). The antibodies tested were shown to cause a significant reduction in proliferation of CD3 positive cells relative to CD3 negative cells. OKT-3 was included as a control and showed comparable cytotoxicity. These results indicated that anti-CD3 mAbs could mediate internalization of the secondary conjugated mAbs and possibly used as cargo for CD3 expressing cells.

TABLE 5
Evaluation of anti-CD3 mAb internalization using an indirect
ADC surrogate assay.

| | Jurkat | | J.RT3-T3.5 | |
|---|---|---|---|---|
| | IC₅₀ | Span | IC₅₀ | Span |
| Clone | (nM) | (%) | (nM) | (%) |
| F282-2D5 | 0.075 | 97 | 10.4 | 100 |
| F332-1C9 (1A10, 5H8, 8B11) | 0.072 | 94 | 11.9 | 96 |
| F332-1E2 (8C5) | 0.031 | 95 | 10.7 | 97 |
| F332-3E9 (12G4) | 0.050 | 94 | 10.8 | 98 |
| F332-4D6 | 0.038 | 92 | 12.0 | 98 |
| F332-5F2 (6G8, 13A3) | 0.173 | 95 | 9.9 | 95 |
| F332-7G4 (6D7) | 0.072 | 96 | 9.0 | 100 |
| F332-5E12 (7C5, 10A8) | 0.062 | 95 | 9.5 | 101 |
| F332-7C9 (5C7, 7D10) | 0.045 | 96 | 10.5 | 98 |
| OKT-3 recomb.(mouse IgG2b) | 0.057 | 91 | 12.1 | 96 |
| 3E6-2 (anti-GFP) | 12.3 | 97 | 10.3 | 95 |

### Example 8: Antibody Sequencing

The VH and VL chains of each mAbs were sequenced by Next Generation Sequencing (NGS). Briefly, mRNA was extracted from hybridoma clones cell pellet (Dynabeads mRNA Direct kit, ThermoFisher Scientific) and reverse transcribed into cDNA (Maxima H Minus First Strand cDNA with dsDNAse, ThermoFisher Scientific). DNA encoding VH and VL domains was PCR amplified (Q5 Hot Start High-Fidelity DNA Polymerase, NEB) using mixtures of degenerate forward primers annealing in FR1 and a single reverse primer annealing in CH1 (Novagen Mouse Ig Heavy, Kappa and Lambda Primer sets). The resulting amplicons were indexed (TrueSeq LT Indexes, Illumina) and the constructed library was then sequenced on a MiSeq System (MiSeq Reagent Nano Kit v2 (500 cycles), Illumina). The DNA sequence of each VH and VL domains were analysed and then translated in silico. The CDRs sequence were determined using Kabat CDR numbering system.

Sequences of the VH and VL domains as well as the CDR regions are shown in Table 6, while the CDRs are aligned in FIGS. 7A and 7B. The CDRs are generally indicated in bold in the light chain or heavy chain variable regions amino acid sequences. Analysis of the sequence for a consensus binding sequence of the CDR 1-3 regions of the VH and VL chains was conducted using Kabat numbering scheme (Kabat et L 1992, Johnson et al 2004). The results indicated that the CDR regions of both the VH and VL regions of the following monoclonal antibodies are identical: 1) F332-1C9 is identical to −1A10, −5H8 and 8B11, 2) F332-1E2 is identical to 8C5, 3) F332-3E9 is identical to 12G4, 4) F332-5D4 is identical to 5F8, 5) F332-5F2 is identical to 6G8 and 13A3, 6) F332-6D7 is identical to 7G4, 7) F332-7C5 is identical to 5E12 and 10A8 and 8) F332-7C9 is identical to 5C7 and 7D10 and have unique VH CDRs. Clone F332-1C9 share the same VH with clone −6D7 and 2 amino acid difference in VL (FR2 and FR4).

The sequence analysis revealed that clones 5C7 (7C9, 7D10), 7B7 and 13B4 have unique VH CDRs. Clones 5D4 (5F8) and 5F2 (6G8, 13A3) share the same CDRH1, CDRH2 and CDRH3. That CDRH1 is also shared in clones 2D5, 1C9 (1A10, 5H8, 8B111) and 5E12 (7C5, 10A8), and that CDRH3 is shared with clone 10B6. Clones 1E2 (8C5), 3E9 (12G4), 4D6 and 7E3 share the same CDRH1 and CDRH3. That CDRH1 is also shared in clones 8F7 and 12F6. Clones 1C9 (1A10, 5H8, 8B11), 8F7 and 12F6 share the same CDRH3. Except for clones 5D4 (5F8) and 5F2 (6G8, 13A3), all CDRH2 are unique. The observed CDRH2 sequences also exhibits marked similarity.

### CDRH Consensus Sequences:

CDRH1:
S/N/D/T-Y/H/S-W/Y-I/M/L/V-H
CDRH2:
N/W-I-N/Y-P-S/N/G-N/S/G/D-G/T-G/T/S/N/D/I-T/I/S-
N/R/K-Y/C-N-E/Q-N/K/R-F-K/T-T/S/N/R/D/G
CDRH3:
D-I/T/R/S/A-S/G-[G]-[Q/S]-Y-Y/F-F-D/N-Y/F

In the above, square brackets [ ] indicate a position that may be absent.

Most of the antibodies share the same CDRL1 (14/15) (KSSQSLLNSRTRKNYLA) and CDRL2 (13/15) (WASTRES). Those that do not share identical CDRL1 and CDRL2 sequences differ at only one residue. All CDRL3 have the same length (8 AA), with similar sequence (X₁-Q-S-X₂-X₃-L-R-T) . . . where X₁ is K or I or T, X₂ is Y or F, and X₃ is H or S or N or I or T.

### CDRL Consensus Sequences:

CDRL1:
K-S-S-Q-S-L-L-N-S-R-T-R-K-N/S-Y-L-A
CDRL2:
W-A-S-T-R-E/K-S
CDRL3:
K/I/T-Q-S-Y/F-H/S/I/N/T-L-R-T

The significant number of identical CDR sequences, together with the high degree of sequence identity in non-identical sequence and the various combinations in which CDRs and variants have been observed in antibodies strongly suggest in this case that specific observed CDR sequences, and individual observed sequence variations therein, should be functionally interchangeable for many applications, though variance in immunochemical properties may show functional variance is some applications (resultant sequences combinations could be readily tested for CD3 binding).

### Example 9: Evaluation of CD3 as Bispecific Antibody

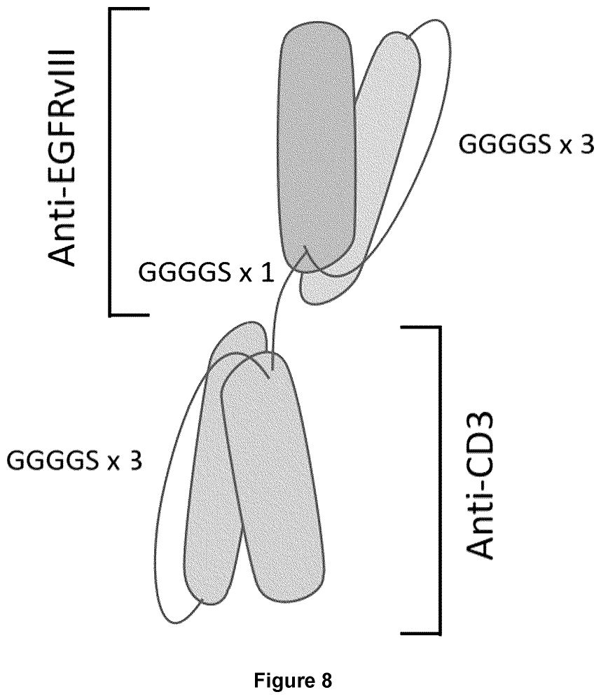

The activity of CD3-specific monoclonal antibodies in multi-antigen binding format were tested by generating molecules containing single-chain variable fragments (scFv) for a CD3-specific T cell targeting arm and antigen targeting arm. See FIG. 8 for an exemplary structure of a bi-specific T cell engager molecule simultaneously targeting CD3 and the tumour specific EGFR-variant Ill (EGFRvIII) antigen. A molecule expressing either an scFv derived from a control CD3 targeting antibody (OKT3) or novel CD3 targeting antibody disclosed herein (F332-1E2, referred to as 1E2 below) were generated similarly as shown in FIG. 8 through synthesis of novel DNA and integration into a plasmid backbone designed for expression within mammalian cells. Proteins were then generated by transient transfection of plasmid DNA into HEK293 cells and collection of culture supernatants after several days.

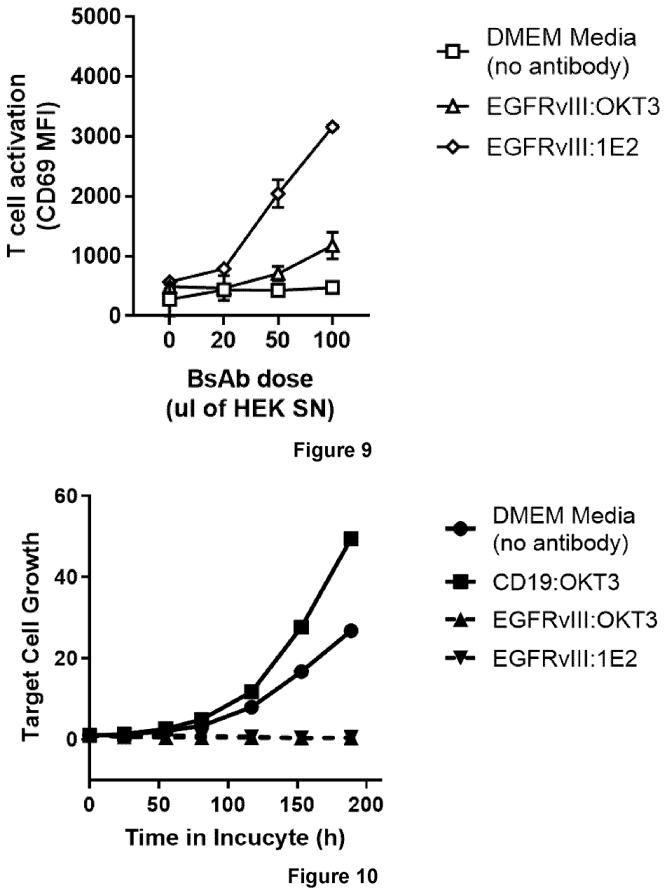

Rough supernatants were then screened for biological activity using co-culture with human Jurkat cells and appropriate antigen expressing target cells. For data shown in FIG. 9, BsAb containing supernatants were generated for previously disclosed EGFRvIII-specific scFv combined with a novel CD3-specific scFv disclosed here (1E2; SEQ ID 40) or a control scFv molecules based on OKT3. After overnight incubation of varying doses of BsAb supernatants with the human Jurkat T cell line and EGFRvIII-expressing U87vIII cells, the level of Jurkat cell activation was assessed by staining with fluorescently labelled anti-human CD69 antibody and flow cytometry. Results show a clear dose dependent increase in activation of Jurkat cells in the presence of EGFRvIII-CD3 BsAb molecules, which is comparable to OKT3 containing BsAbs.

The activity of EGFRvIII-CD3 BsAb molecules were further confirmed in a co-culture assay wherein healthy donor blood derived primary T cells were combined with red fluorescent-protein and EGFRvIII-expressing U87vIII glioblastoma cells. Target cell growth was then monitored within co-cultures using fluorescence microscopy at regular intervals using an Incucyte device (Sartorius USA). As shown in FIG. 10, co-culture in the presence of EGFRvIII-OKT3 or EGFRvIII−1E2 BsAb molecules induced T cell specific responses and similar potent target cell killing, but similar effect was not observed with a control CD19:CD3 specific BsAb. These results demonstrate biological activity of novel EGFRvIII-CD3 BsAb molecules for induction of antigen specific responses.

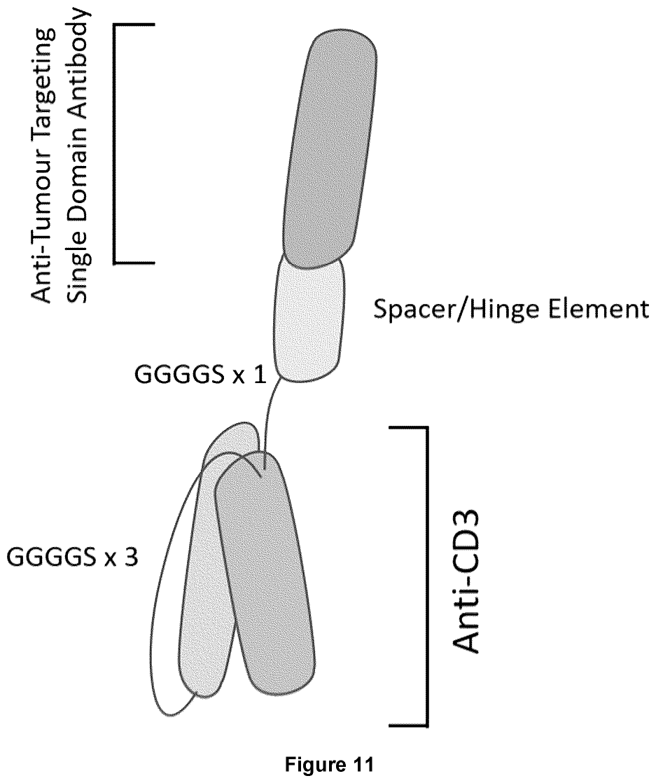

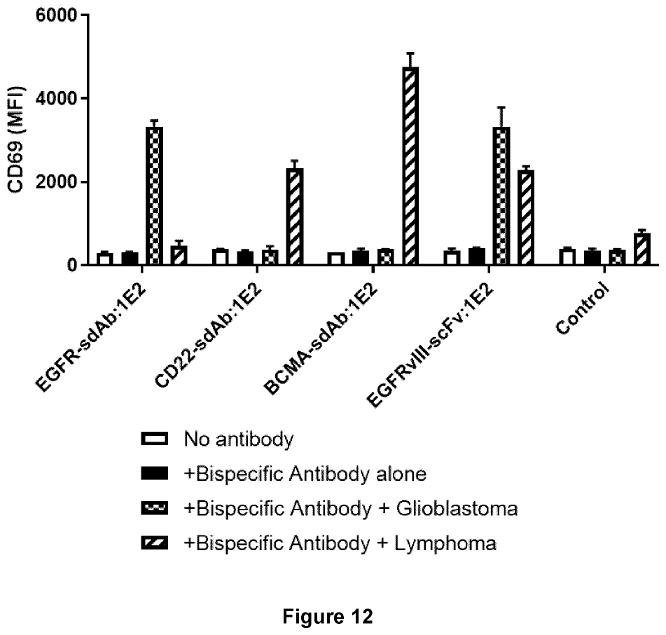

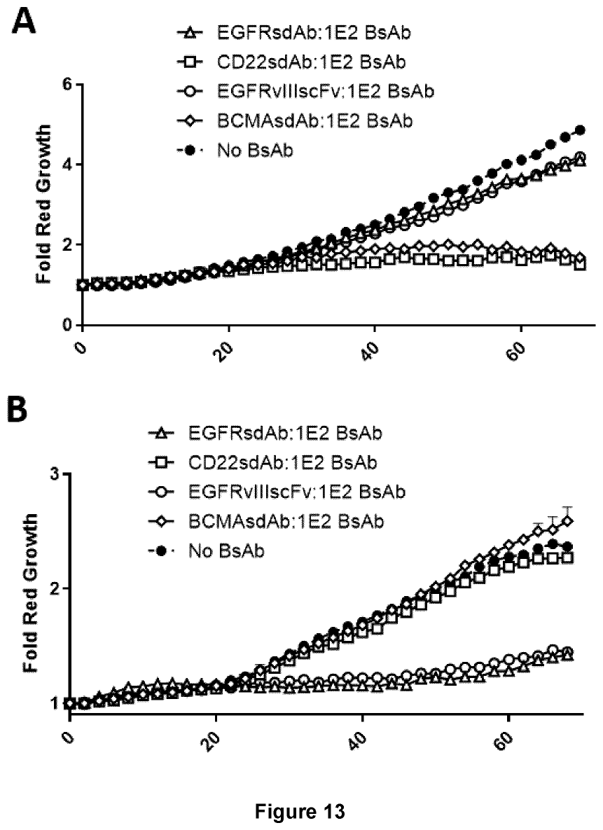

In order to assess the biological activity of alternate molecular formats of BsAbs based on the novel 1E2 CD3 binding elements, plasmids were generated encoding BsAbs with camelid single-domain antibody elements targeting EGFR, CD22, or BCMA similarly as described above; see FIG. 11. An exemplary BsAb targeting human CD3 and human EGFR is provided in SEQ ID 41. BsAb containing supernatants were then generated using HEK293 cells as described above and tested for biological activity using Jurkat cells in co-culture with CD22+/BCMA+/EGFR−/EGFRvIII− lymphoma cells (Ramos) or CD22−/BCMA−/EGFR+/EGFRvIII+ glioblastoma cells (U87vIII). After overnight co-incubation of Jurkat cells, target cells, and BsAb supernatants, Jurkat cell activation was assessed using CD69 antibody staining and flow cytometry. Results shown in FIG. 12 demonstrate antigen specific T cell activation of sdAb-1E2 BsAb molecules targeting EGFR, CD22, or BCMA. Similarly as described above, the activity of sdAb-1E2 BsAb molecules was also assessed for induction of target cell killing with primary human T cell co-cultures as described above. Results shown in FIG. 13 demonstrate that CD22 or BCMA-targeted BsAb can induce potent restriction of lymphoma cell growth (FIG. 13, panel A) but did not affect glioblastoma cell growth (FIG. 13, panel B) whereas EGFR or EGFRvIII targeted BsAbs limited glioblastoma but not lymphoma growth.

### Example 10: CD3-Specific Delivery of Nucleic Acid Cargo

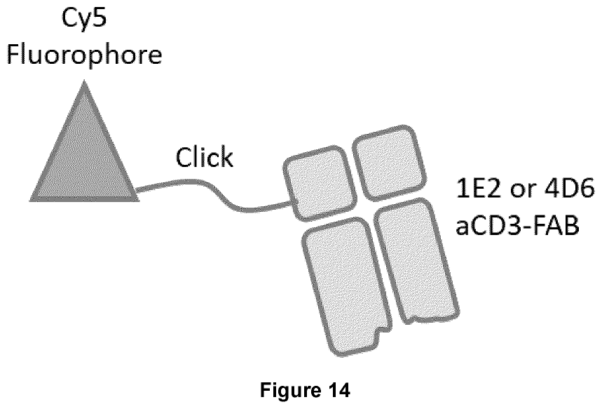

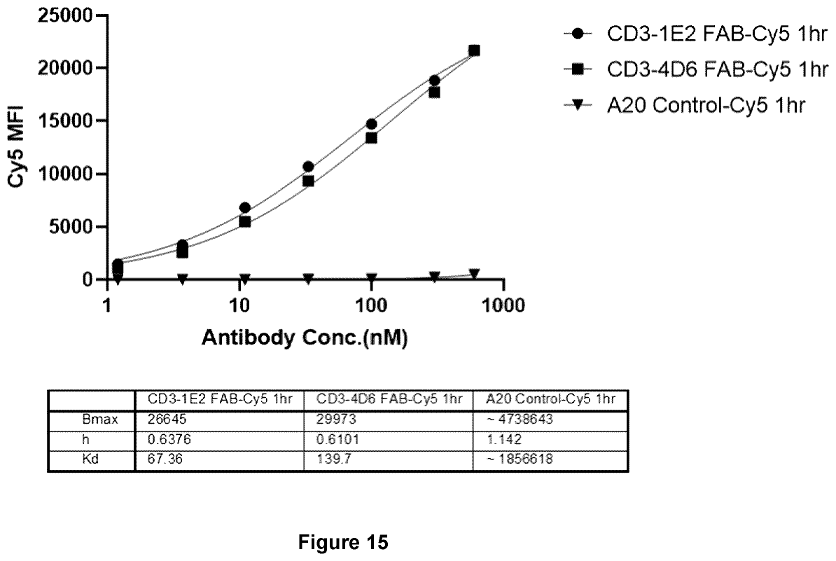

Results shown in Example 7 demonstrate that the monoclonal antibody molecules disclosed here can induce internalization within human CD3-expressing cells. Based on this, it was desirable to test whether such molecules can be applied to deliver nucleic acid cargo to CD3-expressing target cells. In order to generate CD3-targeting molecules more compatible with nucleic acid delivery technology, antigen binding fragment (Fab) molecules were synthesized based on the F332-1E2 and F332-4D6 (referred to as 1E2 or 4D6 respectively below) monoclonal antibodies disclosed here which also incorporated an artificial glycosylation sequon within the antibody heavy chains to provide a consistent c-terminal glycosylation site in the molecule produced. The resultant Fab molecules would be made up of a light chain (SEQ ID 44 or 46) and an o-glycosylated antibody heavy chain (SEQ ID 45 or 47). A enzymatic modification of the glycosylated Fab was then performed to leave a single click-chemistry compatible moiety on the c-terminal end of the Fab molecules which allows site-specific downstream conjugation (see FIG. 14). To then confirm CD3 binding, 1E2 or 4D6 glyco-modified Fabs were conjugated to a click reactive Cy5 fluorescent tag and tested for cell binding with human Jurkat T cells using typical binding conditions with varying antibody concentrations. FIG. 15 shows cell binding for 1E2 or 4D6 Cy5-Fab molecules, but not a non-CD3 specific Cy5 labelled control molecule A20-Cy5.

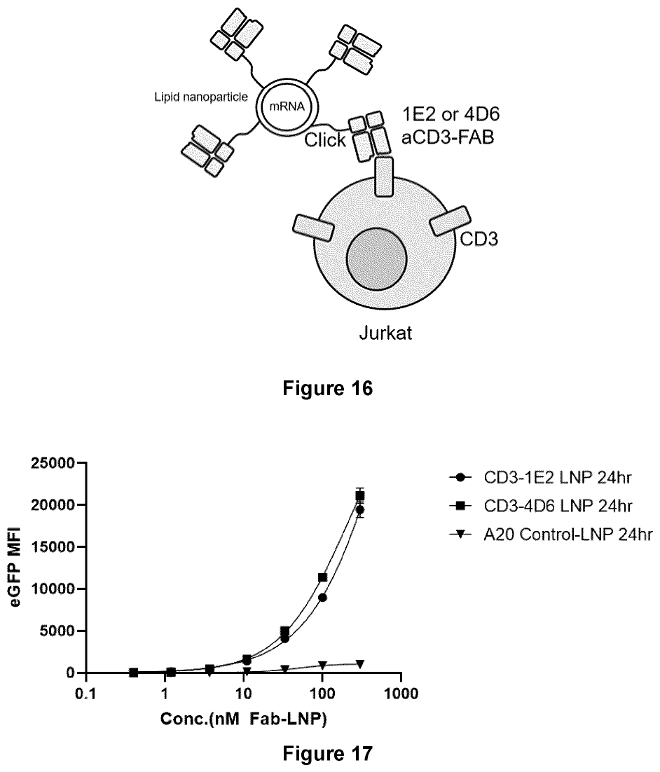

To test whether such glyco-modified Fab molecules can be used to specifically deliver nucleic acid cargo to CD3 cells, 1E2, 4D6, or non-specific control molecules were conjugated to lipid nanoparticles (LNPs). The CD3 binding Fabs were decorated at a specific ratio on the surface of pre-formed LNPs, which were composed of defined ratios of an ionizable cationic lipid, cholesterol, neutral lipid, cholesterol, PEGylated lipid and EGFP mRNA (EGFP-LNP; see a schematic in FIG. 16). Results shown in FIG. 17 demonstrate that incubation of CD3 expressing Jurkat cells with CD3 targeted EGFP mRNA-LNP for 24 hours resulted in strong, dose-dependent expression of EGFP. In contrast, incubation with non-CD3 targeted A20 EGFP-LNP resulted in lower expression of EGFP.

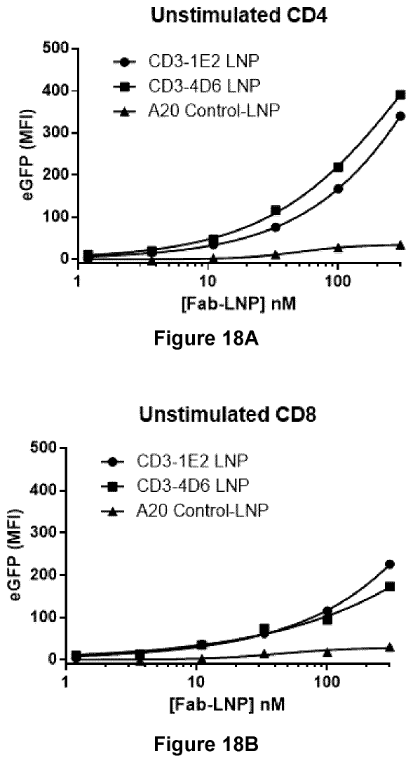

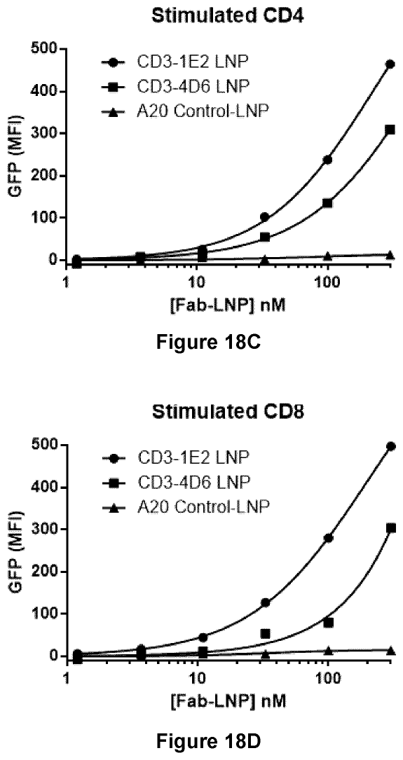

Next it was desirable to see if the CD3-LNPs could similarly target and precipitate mRNA directed EGFP production in primary human T cells. Two types of primary human T cells were employed for these studies: (1) Pure T cells that were activated with T Cell TransAct (Miltenyi Biotech) and maintained in culture for 13 days prior to the CD3-LNP co-culture experiments and (2) similar pure T cells that did not receive T Cell TransAct treatment. Graded amounts of the CD3-1E2, CD3-4D6, and the non-CD3 A20 LNPs containing EGFP mRNA were co-cultured (37° C./5% CO₂) with the activated or non-activated human T cells for a 24 hour period. Following washing, the T cells were stained with anti-human CD4 and CD8 fluorochrome labelled antibodies to distinguish the T cell subsets and level of subset specific EGFP production was determined by flow cytometry (FIGS. 18A to 18D). Results demonstrated that both the CD3-1E2 and CD3-4D6 LNPs were able to elicit a dose dependent production of EGFP in activated and importantly, non-activated primary human T cells. There was no clear discrimination between CD4 and CD8 T cell subsets as EGFP expression in both were comparable. The level of EGFP expression was slightly enhanced in activated T cells as opposed to non-activated cells, which was also born out in terms of % EGFP positivity analysis (data not shown). There was also a moderate observed advantage of CD3-1E2 LNP versus CD3-4D6 LNP in terms of concentration dependent EGFP expression perhaps relating to parameters such as CD3 Fab affinity. In contrast, all co-cultures with the non-CD3 A20 EGFP LNP control resulted in no appreciable production of EGFP in either T cell subset and regardless of the initial T cell activation state.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

Smith, T., Stephan, S., Moffett, H. et al. In situ programming of leukaemia-specific T cells using synthetic DNA nanocarriers. Nature Nanotech 12, 813-820 (2017).
Rasaiyaah J, Georgiadis C, Preece R, Mock U, Qasim W. TCRαβ/CD3 disruption enables CD3-specific antileukemic T cell immunotherapy. JCI Insight. 2018; 3(13):e99442.
Bloemberg et al. Self-Cutting and Integrating CRISPR Plasmids Enable Targeted Genomic Integration of Genetic Payloads for Rapid Cell Engineering. The CRISPR Journal 4(1): 104-119.

TABLE 6
Table of Sequences

| Seq. # | Description | | Sequence |
|---|---|---|---|
| 1 | F282-2D5-2 | Heavy | QVQLQQPGTELVKPGASVKLSCKASGYTFTSYWMHW |
| | | Chain | VKQRPGQGLEWIGNINPSNGGTNYNENFKTKATLTV |
| | | | DKSSSTAYMQLSSLTSEDSAVYYCARDRSGQYYFDY |
| | | | WGQGTTLTVSS |
| 2 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCIQSYHLRTFGGGTI |
| | | | LEIK |
| 3 | >F332-1C9-2, | Heavy | QVQLQQPGAELGKPGTSVNLSCKASGYTFTSYWMHW |
| | F332-1A10-2, | Chain | VKQRPGQGLDWIGNINPNSGTTNYNEKFKSKATLTV |
| | F332-5H8-2 & | | DKSSSTAYMQLSTLTSEDSAVYYCTRDISGQYYFDY |
| | F332-8B11-2 | | WGQGTSLTVSS |
| 4 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLIISSVQAEDLAVYYCKQSYSLRTFGGGTK |
| | | | LEIK |
| 5 | >F332-1E2-2 & | Heavy | QVQLQQPGAELGKPGTSVKLSCKASGYTFTSYWIHW |
| | F332-8C5-2 | Chain | VKQRPGQGLEWVGNINPNSGSINYNEKFKNKATLTV |
| | | | DKSSSTAYMQLSTLTSEDFAVYYCTRDTSGQYYFDY |
| | | | WGQGTTLTVSS |
| 6 | | Light Chain | DIVMSQSPSSLAVSAGERVTMSCKSSQSLLNSRTRK |
| | | | NYLAWYQQKPGQSPKLLIYWASTRESGVPDRFTGSG |
| | | | SGTDFTLTISSVQAEDLAVYYCIQSYHLRTFGGGTK |
| | | | LEIK |
| 7 | >F332-3E9-2 & | Heavy | QVQLQQPGAELGKPGTSVKLSCKASGYTFTSYWIHW |
| | F332-12G4-2 | Chain | LKQRPGQGLEWIGNINPNGGSINCNEKFTTKATLTI |
| | | | DKSSSTAYMQLSTLTSEDFAVYYCTRDTSGQYYFDY |
| | | | WGQGTTLTVSS |
| 8 | | Light Chain | DIMMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCTQSYHLRTFGGGTK |
| | | | LEIK |
| 9 | >F332-4D6-2 | Heavy | QVQLQQSGAELGKPGTSVKLSCKASGYTFTSYWIHW |
| | | Chain | VKQRPGQGLEWIGNINPNSGSTNYNEKFKRKATLTA |
| | | | DKSSSTAYMQLSTLTSEDFAVYYCTRDTSGQYYFDY |
| | | | WGQGTTLTVSS |
| 10 | | Light Chain | DIVMSQSPSSLAVSAGEKVTMSCKSSQSLLNSRTRK |
| | | | NYLAWYQQKPGQSPKVLIYWASTRESGVPDRFTGSG |
| | | | SGTDFTLTISSVQAEDLAVYYCKQSYHLRTFGGGTK |
| | | | LEIK |
| 11 | >F332-5C7-2 & | Heavy | QVQLQQSGPELVKPGASVRISCKASGYTFTSHYLHW |
| | F332-7C9-2 & | Chain | VKQRPGQGFEWIGWIYPGDGNTRYNEKFKDRATLTA |
| | F332-7D10-2 | | DKSSSTAYLHLSSLTSEDSAVYFCARDAGYYFDYWG |
| | | | QGTTLAVSS |
| 12 | | Light Chain | DIVMSQSPSSLAVSAGEKVTMSCKSSQSLLNSRTRK |
| | | | NYLAWYQQKPGQSLKVLIYWASTRESGVPDRFTGSG |
| | | | SGTDFTLTISSVQAEDLAVYYCKQSYSLRTFGGGTK |
| | | | LEIK |
| 13 | >F332-5D4-2 & | Heavy | QVQLQQPGAELGKPGTSVKLSCKASGYTFTSYWMHW |
| | F332-5F8-2 | Chain | VKQRPGQGLEWIGNINPNGGDSNYNEKFKSKATLTV |
| | | | DMSSSTAYMQLSTLTSEDSAVYYCTRDISGQYYFDF |
| | | | WGQGTSLTVES |
| 14 | | Light Chain | DILMSQSPSSLAVSAGEQVTMSCKSSQSLLNSRTRK |
| | | | NYLAWYQQKPGQSPKLLIFWASTRKSGVPDRFTGSG |
| | | | SGTYFTLTINSVQTEDLAVYYCTQSYSLRTFGGGTK |
| | | | LEIK |
| 15 | >F332-5E12-2 & | Heavy | QVQLQQPGAELGKPGTSVKLSCKASGYTFTSYWMHW |
| | F332-7C5-2 & | Chain | VKQRPGQGLEWIGNINPSSGITNYNERFKNKATLTV |
| | F332-10A8-2 | | DKSSTTAYMQLSILTSEDSAVYYCSRDISGQYYFNF |
| | | | WGQGTTLTVSS |
| 16 | | Light Chain | DIVMSQSPSSLAVSAGEKVTMSCKSSQSLLNSRTRK |
| | | | NYLAWYQQKPGQSPKLLIYWASTRESGVPDRETGTG |
| | | | SGTDFTLTISSVQAEDLAVYYCKQSYNLRTFGGGTK |
| | | | LEIK |
| 17 | >F332-5F2-2 & | Heavy | QVQLQQPGAELGKPGTSVKLSCKASGYTFTSYWMHW |
| | F332-6G8-2 & | Chain | VKQRPGQGLEWIGNINPNGGDSNYNEKFKSKATLTV |
| | F332-13A3-2 | | DKSSSTAYMQLSTLTSEDSAVYYCTRDISGQYYFDF |
| | | | WGQGTSLTVES |
| 18 | | Light Chain | DILMSQSPSSLAVSAGEQVTMSCKSSQSLLNSRTRK |
| | | | NYLAWYQQKPGQSPKLLIFWASTRKSGVPDRFTGSG |
| | | | SGTYFTLTISSVQAEDLAVYYCTQSYNLRTFGGGTK |
| | | | LEIK |
| 19 | >F332-6D7-2 & | Heavy | QVQLQQPGAELGKPGTSVNLSCKASGYTFTSYWMHW |
| | F332-7G4-2 | Chain | VKQRPGQGLDWIGNINPNSGTTNYNEKFKSKATLTV |
| | | | DKSSSTAYMQLSTLTSEDSAVYYCTRDISGQYYFDY |
| | | | WGQGTSLTVSS |
| 20 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCKQSYSLRTFGGGSK |
| | | | LEIK |
| 21 | >F332-7B7-2 | Heavy | QVQLQQPGTELGKPGTSVKLSCKASGYTFTNYWMHW |
| | | Chain | VKQRPGQGLEWIGNINPNSGSTNYNQKFKSKATLTL |
| | | | DKSSSTAYMQFSTLTSEDSAVYYCTRDSSGQYYFDY |
| | | | WGQGTTLTVSS |
| 22 | | Light Chain | DIVMSQSPSSLAVSAGEKVTMSCKSSQSLLNSRTRK |
| | | | NYLAWYQQKPGQSPKLLIYWASTRESGVPDRETGSG |
| | | | SGTDFTLTISSVQAEDLAVYYCIQSFHLRTFGGGTK |
| | | | LEIK |
| 23 | >F332-7E3-2 | Heavy | QVQLQQPGAELGKPGTSVKLSCKASGYTFTSYWIHW |
| | | Chain | VKQRPGQGLEWIGNINPNSGSINYNEKFKSKATLTV |
| | | | DKSSSTAYMQLSTLTSEDFAVYYCTRDTSGQYYFDY |
| | | | WGQGTTLTVSS |
| 24 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCKQSYHLRTFGGGTK |
| | | | LEIK |
| 25 | >F332-10B6-2 & | Heavy | QVQLQQPGAELGKPGTSVNLSCKASGYTFTDYWIHW |
| | F332-12B5-1 | Chain | VKQRPGQGLEWIGNINPNSGDTNYNENFKTKATLTV |
| | | | DRSSSTAYMQLSSLTSEDSAVYYCTRDISGQYYFDF |
| | | | WGQGTTLTVSS |
| 26 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCIQSYNLRTFGGGTK |
| | | | LEIK |
| 27 | >F332-12F6-2 | Heavy | QVQLQQPGAELGKPGTSVKLSCKASGYTFTSYWIHW |
| | | Chain | VKQRPGQGLEWIGNINPNSGSTNYNEKFKSKATLTV |
| | | | DKSSSTAYMQLSTLTSEDSAVYYCTRDISGQYYFDY |
| | | | WGQGTTLTVSS |
| 28 | | Light Chain | DIVMSQSPSSLAVSAGEKVTMSCKSSQSLLNSRTRK |
| | | | SYLAWYQQKPGQSPKLLIYWASTRESGVPDRFTGSG |
| | | | SGTDFTLTISSVQAEDLAVYYCIQSYNLRTFGGGTK |
| | | | LEIK |
| 29 | >F332-13B4-2 | Heavy | QVQLQQSGPELVKPGASVKISCKASGYSFTNSYVHW |
| | | Chain | VKQTPGQGLEWIGWIYPGSTNIKYNEKFKGKATLTA |
| | | | DTSSSTVYMQLSSLTSEDSAVYFCARDGSYFFDYWG |
| | | | QGTTLTVSS |
| 30 | | Light Chain | DIVMSQSPSSLAVSAGEKVTMTCKSSQSLLNSRTRK |
| | | | NYLAWYQQKPGQSPKLLIYWASTRESGVPDRETGSG |
| | | | SGTDFTLTISSVQAEDLAVYYCKQSYILRTFGGGTK |
| | | | LEIK |
| 31 | >F332-8F7-3 | Heavy | QVQLQQPGAELGKPGTSVKLSCKASGYTFTSYWIHW |
| | | Chain | VKQRPGQGLEWIGNINPNSGSTNYNEKFKNKATLTV |
| | | | DKSSSTASMQLSTLTSEDSAVYYCTRDISGQYYFDY |
| | | | WGQGTTLTVSS |
| 32 | | Light Chain | DIVMSQSPSSLAVSTGGEVTMSCKSSQSLLNSRTRK |
| | | | NYLAWYQQKPGQSPKLLIYWASTRESGVPDRFTGSG |
| | | | SGTDFTLTISSVQAEDLAVYYCIQSYSLRTFGGGTK |
| | | | LGIK |
| 33 | >F332-10B9-1, | Heavy | QVQLQQSGPELVKPGASVRIPCKASGYTFTTYYIHW |
| | F332-10C9-1 & | Chain | VKQRPGQGLEWIGWIYPGNGNIKYNEKFKGRATLTA |
| | F332-12A9-1 | | DKSSSTAYMQLSSLTSEDSAVYFCARDAGYYFDYWG |
| | | | QGTTLTVSS |
| 34 | | Light Chain | DIVMSQSPSSLAVSAGEKVTMSCKSSQSLLNSRTRK |
| | | | NYLAWYQQKPGQSPKLLIYWASTRESGVPDRFTGSG |
| | | | SGTDFTLTISSVQAEDLAVYYCKQSYTLRTFGGGTK |
| | | | LEIK |
| 35 | Single chain | Amino acid | DIKLQQSGAELARPGASVKMSCKTSGYTFTRYTMHW |
| | variable | sequence | VKQRPGQGLEWIGYINPSRGYTNYNQKFKDKATLTT |
| | fragment | Linker in | DKSSSTAYMQLSSLTSEDSAVYYCARYYDDHYCLDY |
| | (scFv) OKT3 | bold | WGQGTTLTVSSVEGGSGGSGGSGGSGGVDDIQLTQS |
| | | | PAIMSASPGEKVTMTCRASSSVSYMNWYQQKSGTSP |
| | | | KRWIYDTSKVASGVPYRFSGSGSGTSYSLTISSMEA |
| | | | EDAATYYCQQWSSNPLTFGAGTKLELK |
| 36 | Modularized | Amino acid | MEFGLSWVFLVALFRGVQCTGGSSDIQLTQSPASLA |
| | CD19-OKT3 | sequence | VSLGQRATISCKASQSVDYDGDSYLNWYQQIPGQPP |
| | sequence | Signal | KLLIYDASNLVSGIPPRESGSGSGTDFTLNIHPVEK |
| | | peptide in | VDAATYHCQQSTEDPWTFGGGTKLEIKGGGGSGGGG |
| | | italic | SGGGGSQVQLQQSGAELVRPGSSVKISCKASGYAFS |
| | | Restriction | SYWMNWVKQRPGQGLEWIGQIWPGDGDTNYNGKFKG |
| | | sites and | KATLTADESSSTAYMQLSSLASEDSAVYFCARRETT |
| | | linker | TVGRYYYAMDYWGQGTTVTVSSEDFLGGGGSRDDDI |
| | | sequences | KLQQSGAELARPGASVKMSCKTSGYTFTRYTMHWVK |
| | | are in bold | QRPGQGLEWIGYINPSRGYTNYNQKFKDKATLTTDK |
| | | | SSSTAYMQLSSLTSEDSAVYYCARYYDDHYCLDYWG |
| | | | QGTTLTVSSVEGGSGGSGGSGGSGGVDDIQLTQSPA |
| | | | IMSASPGEKVTMTCRASSSVSYMNWYQQKSGTSPKR |
| | | | WIYDTSKVASGVPYRFSGSGSGTSYSLTISSMEAED |
| | | | AATYYCQQWSSNPLTFGAGTKLELKVVSAS |
| 37 | Single chain | Amino acid | QVQLQQPGAELGKPGTSVKLSCKASGYTFTSYWIHW |
| | variable | sequence | VKQRPGQGLEWVGNINPNSGSINYNEKFKNKATLTV |
| | fragment | Linker in | DKSSSTAYMQLSTLTSEDFAVYYCTRDTSGQYYEDY |
| | (scFv) derived | bold | WGQGTTLTVSSGGGGSGGGGSGGGGSDIVMSQSPSS |
| | from F332-1E2 | | LAVSAGERVTMSCKSSQSLLNSRTRKNYLAWYQQKP |
| | | | GQSPKLLIYWASTRESGVPDRFTGSGSGTDFTLTIS |
| | | | SVQAEDLAVYYCIQSYHLRTFGGGTKLEIK |
| 38 | EGFRvIII- | Amino acid | EVQLQQSGPELVKPGSSVRISCKASGYTFTDYNMDW |
| | specific | sequence | VKQSHGKSLEWIGTINPNNGGTSYNQKFKGKATLTV |
| | single chain | Linker in | DKSSSTAYMELRSLTSEDSAVYYCARVRQLGLWFAY |
| | variable | bold | WGQGTLVTVSAAKTTPPSVYPLAPGSLGGTGGGSGG |
| | fragment | | GGSGGGGSEIVLTQSPALMAASPGEKVTITCSVSSS |
| | (scFv) derived | | ISSSNLHWYQQKSETSPKPWIYGTSNLASGVPVRFS |
| | from F269-3D12 | | GSGSGTSYSLTISSMEAEDAATYYCQQWSSYPLTFG |
| | | | SGTKLEIKRADAAPTVSIFPPSSKLG |
| 39 | EGFRvIII-CD3 | Amino acid | MEFGLSWVFLVALFRGVQCTGEVQLQQSGPELVKPG |
| | (F269-3D12- | sequence | SSVRISCKASGYTFTDYNMDWVKQSHGKSLEWIGTI |
| | OKT3) | Signal | NPNNGGTSYNQKFKGKATLTVDKSSSTAYMELRSLT |
| | Bispecific | peptide in | SEDSAVYYCARVRQLGLWFAYWGQGTLVTVSAAKTT |
| | Antibody | italic | PPSVYPLAPGSLGGTGGGSGGGGSGGGGSEIVLTQS |
| | Sequence | Linker | PALMAASPGEKVTITCSVSSSISSSNLHWYQQKSET |
| | | sequences | SPKPWIYGTSNLASGVPVRESGSGSGTSYSLTISSM |
| | | are in bold | EAEDAATYYCQQWSSYPLTFGSGTKLEIKRADAAPT |
| | | | VSIFPPSSKLGDLGGGGSRDDDIKLQQSGAELARPG |
| | | | ASVKMSCKTSGYTFTRYTMHWVKQRPGQGLEWIGYI |
| | | | NPSRGYTNYNQKFKDKATLTTDKSSSTAYMQLSSLT |
| | | | SEDSAVYYCARYYDDHYCLDYWGQGTTLTVSSVEGG |
| | | | SGGSGGSGGSGGVDDIQLTQSPAIMSASPGEKVTMT |
| | | | CRASSSVSYMNWYQQKSGTSPKRWIYDTSKVASGVP |
| | | | YRFSGSGSGTSYSLTISSMEAEDAATYYCQQWSSNP |
| | | | LTFGAGTKLELKHHHHHH* |
| 40 | EGFRVIII-CD3 | Amino acid | MEFGLSWVFLVALFRGVQCTGEVQLQQSGPELVKPG |
| | (F269-3D12- | sequence | SSVRISCKASGYTFTDYNMDWVKQSHGKSLEWIGTI |
| | F322-1E2) | Signal | NPNNGGTSYNQKFKGKATLTVDKSSSTAYMELRSLT |
| | Bispecific | peptide in | SEDSAVYYCARVRQLGLWFAYWGQGTLVTVSAAKTT |
| | Antibody | italic | PPSVYPLAPGSLGGTGGGSGGGGSGGGGSEIVLTQS |
| | Sequence | Linker | PALMAASPGEKVTITCSVSSSISSSNLHWYQQKSET |
| | | sequences | SPKPWIYGTSNLASGVPVRFSGSGSGTSYSLTISSM |
| | | are in bold | EAEDAATYYCQQWSSYPLTFGSGTKLEIKRADAAPT |
| | | | VSIFPPSSKLGDLGGGGSQVQLQQPGAELGKPGTSV |
| | | | KLSCKASGYTFTSYWIHWVKQRPGQGLEWVGNINPN |
| | | | SGSINYNEKFKNKATLTVDKSSSTAYMQLSTLTSED |
| | | | FAVYYCTRDTSGQYYFDYWGQGTTLTVSSGGGGSGG |
| | | | GGSGGGGSDIVMSQSPSSLAVSAGERVTMSCKSSQS |
| | | | LLNSRTRKNYLAWYQQKPGQSPKLLIYWASTRESGV |
| | | | PDRFTGSGSGTDFTLTISSVQAEDLAVYYCIQSYHL |
| | | | RTFGGGTKLEIKHHHHHH* |
| 41 | EGFR-specific | Amino acid | QVQVQLVESGGGLVQPGGSLRLSCSVSLSDFGSKHL |
| | camelid single | sequence | AWHRQAPGKEREWVALIFSSGATNYANSVKGRFTIS |
| | domain antibody | | RDNSKNTVYLQMNSLKPEDTAVYYCNLVPPSPAWGQ |
| | sequence | | GTQVTVSS |
| | (sdAb021) | | |
| 42 | Human CD8- | | TTTPAPRPPTPAPTIASQPLSLRPEACRPAAGGAVH |
| | alpha hinge | | TRGLDFACD |
| | domain | | |
| 43 | EGFRsdAb-CD3 | Amino acid | MEFGLSWVFLVALFRGVQCTGQVQLVESGGGLVQPG |
| | (sdAb021-F322- | sequence | GSLRLSCSVSLSDEGSKHLAWHRQAPGKEREWVALI |
| | 1E2) | Signal | FSSGATNYANSVKGRFTISRDNSKNTVYLQMNSLKP |
| | Bispecific | peptide in | EDTAVYYCNLVPPSPAWGQGTQVTVSSGRLPLRDDG |
| | Antibody | italic | GGGSGGGGSGGGGSGGTTTPAPRPPTPAPTIASQPL |
| | Sequence | Linker | SLRPEACRPAAGGAVHTRGLDFACDLGGGGSQVQLQ |
| | | sequences | QPGAELGKPGTSVKLSCKASGYTFTSYWIHWVKQRP |
| | | are in bold | GQGLEWVGNINPNSGSINYNEKFKNKATLTVDKSSS |
| | | | TAYMQLSTLTSEDFAVYYCTRDTSGQYYFDYWGQGT |
| | | | TLTVSSGGGGSGGGGSGGGGSDIVMSQSPSSLAVSA |
| | | | GERVTMSCKSSQSLLNSRTRKNYLAWYQQKPGQSPK |
| | | | LLIYWASTRESGVPDRFTGSGSGTDFTLTISSVQAE |
| | | | DLAVYYCIQSYHLRTFGGGTKLEIKHHHHHH* |
| 44 | F322-1E2-Fab | Light chain | DIVMSQSPSSLAVSAGERVTMSCKSSQSLLNSRTRK |
| | Light chain | Amino acid | NYLAWYQQKPGQSPKLLIYWASTRESGVPDRFTGSG |
| | | sequence | SGTDFTLTISSVQAEDLAVYYCIQSYHLRTFGGGTK |
| | | | LEIKRTVAAPSVFIFPPSDEQLKSGTASVVCLLNNF |
| | | | YPREAKVQWKVDNALQSGNSQESVTEQDSKDSTYSL |
| | | | SSTLTLSKADYEKHKVYACEVTHQGLSSPVTKSFNR |
| | | | GEC |
| 45 | F322-1E2-Fab | Heavy | QVQLQQPGAELGKPGTSVKLSCKASGYTFTSYWIHW |
| | Heavy Chain | chain | VKQRPGQGLEWVGNINPNSGSINYNEKFKNKATLTV |
| | | Amino acid | DKSSSTAYMQLSTLTSEDFAVYYCTRDTSGQYYFDY |
| | | sequence | WGQGTTLTVSSASTKGPSVFPLAPSSKSTSGGTAAL |
| | | O-linked | GCLVKDYFPEPVTVSWNSGALTSGVHTFPAVLQSSG |
| | | glycosylation | LYSLSSVVTVPSSSLGTQTYICNVNHKPSNTKVDKK |
| | | sequon in | VEPKSCDGGGGGAPALQPTQGAMPA |
| | | bold | |
| 46 | F322-4D6-Fab | Light chain | DIVMSQSPSSLAVSAGEKVTMSCKSSQSLLNSRTRK |
| | | Amino acid | NYLAWYQQKPGQSPKVLIYWASTRESGVPDRFTGSG |
| | | sequence | SGTDETLTISSVQAEDLAVYYCKQSYHLRTFGGGTK |
| | | | LEIKRTVAAPSVFIFPPSDEQLKSGTASVVCLLNNF |
| | | | YPREAKVQWKVDNALQSGNSQESVTEQDSKDSTYSL |
| | | | SSTLTLSKADYEKHKVYACEVTHQGLSSPVTKSFNR |
| | | | GEC |
| 47 | F322-4D6-Fab | Heavy | QVQLQQSGAELGKPGTSVKLSCKASGYTFTSYWIHW |
| | Heavy Chain | chain | VKQRPGQGLEWIGNINPNSGSTNYNEKFKRKATLTA |
| | | Amino acid | DKSSSTAYMQLSTLTSEDFAVYYCTRDTSGQYYEDY |
| | | sequence | WGQGTTLTVSSASTKGPSVFPLAPSSKSTSGGTAAL |
| | | O-linked | GCLVKDYFPEPVTVSWNSGALTSGVHTFPAVLQSSG |
| | | glycosylation | LYSLSSVVTVPSSSLGTQTYICNVNHKPSNTKVDKK |
| | | sequon in | VEPKSCDGGGGGAPALQPTQGAMPA |
| | | bold | |
| 48 | F282-2D5-2 | CDRH1 | SYWMH |
| 49 | | CDRH2 | NINPSNGGTNYNENFKT |
| 50 | | CDRH3 | DRSGQYYFDY |
| 51 | | CDRL1 | KSSQSLLNSRTRKNYLA |
| 52 | | CDRL2 | WASTRES |
| 53 | | CDRL3 | IQSYHLRT |
| 54 | >F332-1C9-2, | CDRH1 | SYWMH |
| | F332-1A10-2, | | |
| | F332-5H8-2 & | | |
| | F332-8B11-2 | | |
| 55 | | CDRH2 | NINPNSGTTNYNEKFKS |
| 56 | | CDRH3 | DISGQYYFDY |
| 57 | | CDRL1 | KSSQSLLNSRTRKNYLA |
| 58 | | CDRL2 | WASTRES |
| 59 | | CDRL3 | KQSYSLRT |
| 60 | >F332-1E2-2 & | CDRH1 | SYWIH |
| | F332-8C5-2 | | |
| 61 | | CDRH2 | NINPNSGSINYNEKFKN |
| 62 | | CDRH3 | DTSGQYYFDY |
| 63 | | CDRL1 | KSSQSLLNSRTRKNYLA |
| 64 | | CDRL2 | WASTRES |
| 65 | | CDRL3 | IQSYHLRT |
| 66 | >F332-3E9-2 & | CDRH1 | SYWIH |
| | F332-12G4-2 | | |
| 67 | | CDRH2 | NINPNGGSINCNEKFTT |
| 68 | | CDRH3 | DTSGQYYFDY |
| 69 | | CDRL1 | KSSQSLLNSRTRKNYLA |
| 70 | | CDRL2 | WASTRES |
| 71 | | CDRL3 | TQSYHLRT |
| 72 | >F332-4D6-2 | CDRH1 | SYWIH |
| 73 | | CDRH2 | NINPNSGSTNYNEKFKR |
| 74 | | CDRH3 | DTSGQYYFDY |
| 75 | | CDRL1 | KSSQSLLNSRTRKNYLA |
| 76 | | CDRL2 | WASTRES |
| 77 | | CDRL3 | KQSYHLRT |
| 78 | >F332-5C7-2 & | CDRH1 | SHYLH |
| | F332-7C9-2 & | | |
| | F332-7D10-2 | | |
| 79 | | CDRH2 | WIYPGDGNTRYNEKFKD |
| 80 | | CDRH3 | DAGYYFDY |
| 81 | | CDRL1 | KSSQSLLNSRTRKNYLA |
| 82 | | CDRL2 | WASTRES |
| 83 | | CDRL3 | KQSYSLRT |
| 84 | >F332-5D4-2 & | CDRH1 | SYWMH |
| | F332-5F8-2 | | |
| 85 | | CDRH2 | NINPNGGDSNYNEKFKS |
| 86 | | CDRH3 | DISGQYYFDE |
| 87 | | CDRL1 | KSSQSLLNSRTRKNYLA |
| 88 | | CDRL2 | WASTRKS |
| 89 | | CDRL3 | TQSYSLRT |
| 90 | >F332-5E12-2 & | CDRH1 | SYWMH |
| | F332-7C5-2 & | | |
| | F332-10A8-2 | | |
| 91 | | CDRH2 | NINPSSGITNYNERFKN |
| 92 | | CDRH3 | DISGQYYENF |
| 93 | | CDRL1 | KSSQSLLNSRTRKNYLA |
| 94 | | CDRL2 | WASTRES |
| 95 | | CDRL3 | KQSYNLRT |
| 96 | >F332-5F2-2 & | CDRH1 | SYWMH |
| | F332-6G8-2 & | | |
| | F332-13A3-2 | | |
| 97 | | CDRH2 | NINPNGGDSNYNEKFKS |
| 98 | | CDRH3 | DISGQYYFDF |
| 99 | | CDRL1 | KSSQSLLNSRTRKNYLA |
| 100 | | CDRL2 | WASTRKS |
| 101 | | CDRL3 | TQSYNLRT |
| 102 | >F332-6D7-2 & | CDRH1 | SYWMH |
| | F332-7G4-2 | | |
| 103 | | CDRH2 | NINPNSGTTNYNEKFK |
| 104 | | CDRH3 | DISGQYYFDY |
| 105 | | CDRL1 | KSSQSLLNSRTRKNYLA |
| 106 | | CDRL2 | WASTRES |
| 107 | | CDRL3 | KQSYSLRT |
| 108 | >F332-7B7-2 | CDRH1 | NYWMH |
| 109 | | CDRH2 | NINPNSGSTNYNQKFKS |
| 110 | | CDRH3 | DSSGQYYFDY |
| 111 | | CDRL1 | KSSQSLLNSRTRKNYLA |
| 112 | | CDRL2 | WASTRES |
| 113 | | CDRL3 | IQSFHLRT |
| 114 | >F332-7E3-2 | CDRH1 | SYWIH |
| 115 | | CDRH2 | NINPNSGSINYNEKFKS |
| 116 | | CDRH3 | DTSGQYYFDY |
| 117 | | CDRL1 | KSSQSLLNSRTRKNYLA |
| 118 | | CDRL2 | WASTRES |
| 119 | | CDRL3 | KQSYHLRT |
| 120 | >F332-10B6-2 & | CDRH1 | DYWIH |
| | F332-12B5-1 | | |
| 121 | | CDRH2 | NINPNSGDTNYNENFKT |
| 122 | | CDRH3 | DISGQYYFDF |
| 123 | | CDRL1 | KSSQSLLNSRTRKNYLA |
| 124 | | CDRL2 | WASTRES |
| 125 | | CDRL3 | IQSYNLRT |
| 126 | >F332-12F6-2 | CDRH1 | SYWIH |
| 127 | | CDRH2 | NINPNSGSTNYNEKFKS |
| 128 | | CDRH3 | DISGQYYFDY |
| 129 | | CDRL1 | KSSQSLLNSRTRKSYLA |
| 130 | | CDRL2 | WASTRES |
| 131 | | CDRL3 | IQSYNLRT |
| 132 | >F332-13B4-2 | CDRH1 | NSYVH |
| 133 | | CDRH2 | WIYPGSTNIKYNEKFKG |
| 134 | | CDRH3 | DGSYFFDY |
| 135 | | CDRL1 | KSSQSLLNSRTRKNYLA |
| 136 | | CDRL2 | WASTRES |
| 137 | | CDRL3 | KQSYILRT |
| 138 | >F332-8F7-3 | CDRH1 | SYWIH |
| 139 | | CDRH2 | NINPNSGSTNYNEKFKN |
| 140 | | CDRH3 | DISGQYYFDY |
| 141 | | CDRL1 | KSSQSLLNSRTRKNYLA |
| 142 | | CDRL2 | WASTRES |
| 143 | | CDRL3 | IQSYSLRT |
| 144 | >F332-10B9-1, | CDRH1 | TYYIH |
| | F332-10C9-1 & | | |
| | F332-12A9-1 | | |
| 145 | | CDRH2 | WIYPGNGNIKYNEKFKG |
| 146 | | CDRH3 | DAGYYFDY |
| 147 | | CDRL1 | KSSQSLLNSRTRKNYLA |
| 148 | | CDRL2 | WASTRES |
| 149 | | CDRL3 | KQSYTLRT |
| 150 | Consensus | CDRH1 | X₁X₂X₃X₄H |
| | | | wherein X₁ is S, N, D or T; |
| | | | wherein X₂ is Y, H, or S; |
| | | | wherein X₃ is W or Y; and |
| | | | wherein X₄ is I, M, L or V |
| 151 | Consensus | CDRH2 | X₅IX₆PX₇X₈X₉X₁₀X₁₁X₁₂X₁₃NX₁₄X₁₅FX₁₆X₁₇ |
| | | | wherein X₅ is N or W; |
| | | | wherein X₆ is N or Y; |
| | | | wherein X₇ is S, N, or G; |
| | | | wherein X₈ is N, S, G, or D; |
| | | | wherein X₉ is G or T; |
| | | | wherein X₁₀ is G, T, S, N, D, or I; |
| | | | wherein X₁₁ is T, I, or S; |
| | | | wherein X₁₂ is N, R, or K; |
| | | | wherein X₁₃ is Y or C |
| | | | wherein X₁₄ is E or Q; |
| | | | wherein X₁₅ is N, K, or R; |
| | | | wherein X₁₆ is K or T; and |
| | | | wherein X₁₇ T, S, N, R, D, or G |
| 152 | Consensus | CDRH3 | DX₁₈X₁₉X₂₀X₂₁YX₂₂FX₂₃X₂₄ |
| | | | wherein X₁₈ is I, T, R, S, or A; |
| | | | wherein X₁₉ is S or G; |
| | | | wherein X₂₀ is G or absent; |
| | | | wherein X₂₁ is G, S, or absent; |
| | | | wherein X₂₂ is Y or F; |
| | | | wherein X₂₃ is D or N; and |
| | | | wherein X₂₄ is Y, or F |
| 153 | Consensus | CDRL1 | KSSQSLLNSRTRKX₂₅YLA |
| | | | wherein X₂₅ is N or S |
| 154 | Consensus | CDRL2 | WASTRX ₂₆S |
| | | | wherein X₂₆ is E or K |
| 155 | Consensus | CDRL3 | X₂₇QSX₂₈X₂₉LRT |
| | | | wherein X₂₇ is K, I, or T; |
| | | | wherein X₂₈ is Y or F; and |
| | | | wherein X₂₉ is H, S, I, N, or T |
| 156 | Signal Peptide | | MEFGLSWVELVALERGVQCTG |
| 157 | Artificial | | GGGGGAPALQPTQGAMPA |
| | Glycosylation | | |
| | Sequon | | |
| 158 | Human CD8 | | TTTPAPRPPTPAPTIASQPLSLRPEACRPAAGGAVHTRGL |
| | Hinge Domain | | DFACD |
| 159 | Human CD28 | | PSKPFWVLVVVGGVLACYSLLVTVAFIIFWVR |
| | Transmembrane | | |
| | Domain | | |
| 160 | Human CD3zeta | | LRVKFSRSADAPAYQQGQNQLYNELNLGRREEYDVLDKRR |
| | Signaling Domain | | GRDPEMGGKPQRRKNPQEGLYNELQKDKMAEAYSEIGMKG |
| | | | ERRRGKGHDGLYQGLSTATKDTYDALHMQALPPR |
| 161 | Human 4-1BB Co- | | KRGRKKLLYIFKQPFMRPVQTTQEEDGCSCRFPEEEEGGC |
| | stimulatory | | EL |
| | Domain | | |
| 162 | Flexible linker | | GGGGSGGGGSGGGGSGG |
| 163 | Human CD28 | | MLRLLLALNLFPSIQVTG |
| | Signal Peptide | | |
| 164 | Linker | | GGGGS |
| 165 | Linker | | GGGGSGGGGSGGGGS |

1. An isolated or purified monoclonal antibody, or an antigen-binding fragment thereof, which binds to human CD3, and which comprises:
a CDRH1 amino acid sequence of SEQ ID NO: 150,
a CDRH2 amino acid sequence of SEQ ID NO: 151,
a CDRH3 amino acid sequence of SEQ ID NO: 152,
a CDRL1 amino acid sequence of SEQ ID NO: 153,
a CDRL2 amino acid sequence of SEQ ID NO: 154, and
a CDRL3 amino acid sequence of SEQ ID NO: 155.

2. An isolated or purified monoclonal antibody, or an antigen-binding fragment thereof, which binds to human CD3, and which comprises:
a CDRH1 amino acid sequence selected from the group consisting of SEQ ID NOs: 48, 54, 60, 66, 72, 78, 84, 90, 96, 102, 108, 114, 120, 126, 132, 138, and 144,
a CDRH2 amino acid sequence selected from the group consisting of SEQ ID NOs: 49, 55, 61, 67, 73, 79, 85, 91, 97, 103, 109, 115, 121, 127, 133, 139, and 145,
a CDRH3 amino acid sequence selected from the group consisting of SEQ ID NOs: 50, 56, 62, 68, 74, 80, 86, 92, 98, 104, 110, 116, 122, 128, 134, 140, and 146,
a CDRL1 amino acid sequence selected from the group consisting of SEQ ID NOs: 51, 57, 63, 69, 75, 81, 87, 93, 99, 105, 111, 117, 123, 129, 135, 141, and 147,
a CDRL2 amino acid sequence selected from the group consisting of SEQ ID NOs: 52, 58, 64, 70, 76, 82, 88, 94, 100, 106, 112, 118, 124, 130, 136, 142, and 148, and
a CDRL3 amino acid sequence selected from the group consisting of SEQ ID NOs: 53, 59, 65, 71, 77, 83, 89, 95, 101, 106, 113, 119, 125, 130, 136, 143, and 149.

3. An isolated or purified monoclonal antibody, or an antigen-binding fragment thereof, which binds to human CD3, and which comprises:
A)
i) a CDRH1 amino acid sequence of SEQ ID NO: 48,
a CDRH2 amino acid sequence of SEQ ID NO: 49,
a CDRH3 amino acid sequence of SEQ ID NO: 50,
a CDRL1 amino acid sequence of SEQ ID NO: 51,
a CDRL2 amino acid sequence of SEQ ID NO: 52, and
a CDRL3 amino acid sequence of SEQ ID NO: 53;
ii) a CDRH1 amino acid sequence of SEQ ID NO: 54,
a CDRH2 amino acid sequence of SEQ ID NO: 55,
a CDRH3 amino acid sequence of SEQ ID NO: 56,
a CDRL1 amino acid sequence of SEQ ID NO: 57,
a CDRL2 amino acid sequence of SEQ ID NO: 58, and
a CDRL3 amino acid sequence of SEQ ID NO: 59;
iii) a CDRH1 amino acid sequence of SEQ ID NO: 60,
a CDRH2 amino acid sequence of SEQ ID NO: 61,
a CDRH3 amino acid sequence of SEQ ID NO: 62,
a CDRL1 amino acid sequence of SEQ ID NO: 63,
a CDRL2 amino acid sequence of SEQ ID NO: 64, and
a CDRL3 amino acid sequence of SEQ ID NO: 65;
iv) a CDRH1 amino acid sequence of SEQ ID NO: 66,
a CDRH2 amino acid sequence of SEQ ID NO: 67,
a CDRH3 amino acid sequence of SEQ ID NO: 68,
a CDRL1 amino acid sequence of SEQ ID NO: 69,
a CDRL2 amino acid sequence of SEQ ID NO: 70, and
a CDRL3 amino acid sequence of SEQ ID NO: 71;
v) a CDRH1 amino acid sequence of SEQ ID NO: 72,
a CDRH2 amino acid sequence of SEQ ID NO: 73,
a CDRH3 amino acid sequence of SEQ ID NO: 74,
a CDRL1 amino acid sequence of SEQ ID NO: 75,
a CDRL2 amino acid sequence of SEQ ID NO: 76, and
a CDRL3 amino acid sequence of SEQ ID NO: 77;
vi) a CDRH1 amino acid sequence of SEQ ID NO: 78,
a CDRH2 amino acid sequence of SEQ ID NO: 79,
a CDRH3 amino acid sequence of SEQ ID NO: 80,
a CDRL1 amino acid sequence of SEQ ID NO: 81,
a CDRL2 amino acid sequence of SEQ ID NO: 82, and
a CDRL3 amino acid sequence of SEQ ID NO: 83;
vii) a CDRH1 amino acid sequence of SEQ ID NO: 84,
a CDRH2 amino acid sequence of SEQ ID NO: 85,
a CDRH3 amino acid sequence of SEQ ID NO: 86,
a CDRL1 amino acid sequence of SEQ ID NO: 87,
a CDRL2 amino acid sequence of SEQ ID NO: 88, and
a CDRL3 amino acid sequence of SEQ ID NO: 89;
viii) a CDRH1 amino acid sequence of SEQ ID NO: 90,
a CDRH2 amino acid sequence of SEQ ID NO: 91,
a CDRH3 amino acid sequence of SEQ ID NO: 92,
a CDRL1 amino acid sequence of SEQ ID NO: 93,
a CDRL2 amino acid sequence of SEQ ID NO: 94, and
a CDRL3 amino acid sequence of SEQ ID NO: 95;
ix) a CDRH1 amino acid sequence of SEQ ID NO: 96,
a CDRH2 amino acid sequence of SEQ ID NO: 97,
a CDRH3 amino acid sequence of SEQ ID NO: 98,
a CDRL1 amino acid sequence of SEQ ID NO: 99,
a CDRL2 amino acid sequence of SEQ ID NO: 100, and
a CDRL3 amino acid sequence of SEQ ID NO: 101;
x) a CDRH1 amino acid sequence of SEQ ID NO: 102,
a CDRH2 amino acid sequence of SEQ ID NO: 103,
a CDRH3 amino acid sequence of SEQ ID NO: 104,
a CDRL1 amino acid sequence of SEQ ID NO: 10⁵,
a CDRL2 amino acid sequence of SEQ ID NO: 106, and
a CDRL3 amino acid sequence of SEQ ID NO: 107;
xi) a CDRH1 amino acid sequence of SEQ ID NO: 108,
a CDRH2 amino acid sequence of SEQ ID NO: 109,
a CDRH3 amino acid sequence of SEQ ID NO: 110,
a CDRL1 amino acid sequence of SEQ ID NO: 111,
a CDRL2 amino acid sequence of SEQ ID NO: 112, and
a CDRL3 amino acid sequence of SEQ ID NO: 113;
xii) a CDRH1 amino acid sequence of SEQ ID NO: 114,
a CDRH2 amino acid sequence of SEQ ID NO: 115,
a CDRH3 amino acid sequence of SEQ ID NO: 116,
a CDRL1 amino acid sequence of SEQ ID NO: 117,
a CDRL2 amino acid sequence of SEQ ID NO: 118, and
a CDRL3 amino acid sequence of SEQ ID NO: 119;
xiii) a CDRH1 amino acid sequence of SEQ ID NO: 120,
a CDRH2 amino acid sequence of SEQ ID NO: 121,
a CDRH3 amino acid sequence of SEQ ID NO: 122,
a CDRL1 amino acid sequence of SEQ ID NO: 123,
a CDRL2 amino acid sequence of SEQ ID NO: 124, and
a CDRL3 amino acid sequence of SEQ ID NO: 125;
xiv) a CDRH1 amino acid sequence of SEQ ID NO: 126,
a CDRH2 amino acid sequence of SEQ ID NO: 127,
a CDRH3 amino acid sequence of SEQ ID NO: 128,
a CDRL1 amino acid sequence of SEQ ID NO: 129,
a CDRL2 amino acid sequence of SEQ ID NO: 130, and
a CDRL3 amino acid sequence of SEQ ID NO: 131;
xv) a CDRH1 amino acid sequence of SEQ ID NO: 132,
a CDRH2 amino acid sequence of SEQ ID NO: 133,
a CDRH3 amino acid sequence of SEQ ID NO: 134,
a CDRL1 amino acid sequence of SEQ ID NO: 135,
a CDRL2 amino acid sequence of SEQ ID NO: 136, and
a CDRL3 amino acid sequence of SEQ ID NO: 137;
xvi) a CDRH1 amino acid sequence of SEQ ID NO: 138,
a CDRH2 amino acid sequence of SEQ ID NO: 139,
a CDRH3 amino acid sequence of SEQ ID NO: 140,
a CDRL1 amino acid sequence of SEQ ID NO: 141,
a CDRL2 amino acid sequence of SEQ ID NO: 142, and
a CDRL3 amino acid sequence of SEQ ID NO: 143; or
xvii) a CDRH1 amino acid sequence of SEQ ID NO: 144,
a CDRH2 amino acid sequence of SEQ ID NO: 145,
a CDRH3 amino acid sequence of SEQ ID NO: 146,
a CDRL1 amino acid sequence of SEQ ID NO: 147,
a CDRL2 amino acid sequence of SEQ ID NO: 148, and
a CDRL3 amino acid sequence of SEQ ID NO: 149;
OR
B)
CDRH1, CDRH2, CDRH3, CDRL1, CDRL2, and CDRL3 amino acid sequences that are at least 80% identical to the CDRH1, CDRH2, CDRH3, CDRL1, CDRL2, and CDRL3 sequences, respectively, defined in any one of A) i) to xvii).

4. The isolated or purified monoclonal antibody, or the antigen-binding fragment thereof, of claim 3, which is as defined as in A).

5. The isolated or purified monoclonal antibody, or the antigen-binding fragment thereof, of any one of claims 1 to 4, which comprises a heavy chain sequence selected from the group consisting of SEQ ID NOs: 1, 3, 5, 7, 9, 11, 13, 15, 17, 19, 21, 23, 25, 27, 29, 30, 31, and 33.

6. The isolated or purified monoclonal antibody, or the antigen-binding fragment thereof, of any one of claims 1 to 5, which comprises a light chain sequence selected from the group consisting of SEQ ID NOs: 2, 4, 6, 8, 10, 12, 14, 16, 18, 20, 22, 24, 26, 28, 30, 32, and 34.

7. The isolated or purified monoclonal antibody, or the antigen-binding fragment thereof, of any one of claims 1 to 4, which comprises:
A)
a) a heavy chain amino acid sequence of SEQ ID NO: 1 and a light chain amino acid sequence of SEQ ID NO: 2,
b) a heavy chain amino acid sequence of SEQ ID NO: 3 and a light chain amino acid sequence of SEQ ID NO: 4,
c) a heavy chain amino acid sequence of SEQ ID NO: 5 and a light chain amino acid sequence of SEQ ID NO: 6,
d) a heavy chain amino acid sequence of SEQ ID NO: 7 and a light chain amino acid sequence of SEQ ID NO: 8,
e) a heavy chain amino acid sequence of SEQ ID NO: 9 and a light chain amino acid sequence of SEQ ID NO: 10,
f) a heavy chain amino acid sequence of SEQ ID NO: 11 and a light chain amino acid sequence of SEQ ID NO: 12,
g) a heavy chain amino acid sequence of SEQ ID NO: 13 and a light chain amino acid sequence of SEQ ID NO: 14,
h) a heavy chain amino acid sequence of SEQ ID NO: 15 and a light chain amino acid sequence of SEQ ID NO: 16,
i) a heavy chain amino acid sequence of SEQ ID NO: 17 and a light chain amino acid sequence of SEQ ID NO: 18,
j) a heavy chain amino acid sequence of SEQ ID NO: 19 and a light chain amino acid sequence of SEQ ID NO: 20,
k) a heavy chain amino acid sequence of SEQ ID NO: 21 and a light chain amino acid sequence of SEQ ID NO: 22,
l) a heavy chain amino acid sequence of SEQ ID NO: 23 and a light chain amino acid sequence of SEQ ID NO: 24,
m) a heavy chain amino acid sequence of SEQ ID NO: 25 and a light chain amino acid sequence of SEQ ID NO: 26,
n) a heavy chain amino acid sequence of SEQ ID NO: 27 and a light chain amino acid sequence of SEQ ID NO: 28,
o) a heavy chain amino acid sequence of SEQ ID NO: 29 and a light chain amino acid sequence of SEQ ID NO: 30,
p) a heavy chain amino acid sequence of SEQ ID NO: 31 and a light chain amino acid sequence of SEQ ID NO: 32, or
q) a heavy chain amino acid sequence of SEQ ID NO: 33 and a light chain amino acid sequence of SEQ ID NO: 34; or
B)
a heavy chain amino acid sequence at a light chain amino acid sequence that are at least 80% identical to the heavy chain amino acid sequences and the light chain amino acid sequences defined in defined in any one of A) a) to q).

8. The isolated or purified monoclonal antibody, or the antigen-binding fragment thereof, of claim 7, which comprises the heavy chain amino acid sequence of SEQ ID NO: 1 and the light chain amino acid sequence of SEQ ID NO: 2.

9. The isolated or purified monoclonal antibody, or the antigen-binding fragment thereof, of claim 7, which comprises the heavy chain amino acid sequence of SEQ ID NO: 3 and the light chain amino acid sequence of SEQ ID NO: 4.

10. The isolated or purified monoclonal antibody, or the antigen-binding fragment thereof, of claim 7, which comprises the heavy chain amino acid sequence of SEQ ID NO: 5 and the light chain amino acid sequence of SEQ ID NO: 6.

11. The isolated or purified monoclonal antibody, or the antigen-binding fragment thereof, of claim 7, which comprises the heavy chain amino acid sequence of SEQ ID NO: 7 and the light chain amino acid sequence of SEQ ID NO: 8.

12. The isolated or purified monoclonal antibody, or the antigen-binding fragment thereof, of claim 7, which comprises the heavy chain amino acid sequence of SEQ ID NO: 9 and the light chain amino acid sequence of SEQ ID NO: 10.

13. The isolated or purified monoclonal antibody, or the antigen-binding fragment thereof, of claim 7, which comprises the heavy chain amino acid sequence of SEQ ID NO: 11 and the light chain amino acid sequence of SEQ ID NO: 12.

14. The isolated or purified monoclonal antibody, or the antigen-binding fragment thereof, of claim 7, which comprises the heavy chain amino acid sequence of SEQ ID NO: 13 and the light chain amino acid sequence of SEQ ID NO: 14.

15. The isolated or purified monoclonal antibody, or the antigen-binding fragment thereof, of claim 7, which comprises the heavy chain amino acid sequence of SEQ ID NO: 15 and the light chain amino acid sequence of SEQ ID NO: 16.

16. The isolated or purified monoclonal antibody, or the antigen-binding fragment thereof, of claim 7, which comprises the heavy chain amino acid sequence of SEQ ID NO: 17 and the light chain amino acid sequence of SEQ ID NO: 18.

17. The isolated or purified monoclonal antibody, or the antigen-binding fragment thereof, of claim 7, which comprises the heavy chain amino acid sequence of SEQ ID NO: 19 and the light chain amino acid sequence of SEQ ID NO: 20.

18. The isolated or purified monoclonal antibody, or the antigen-binding fragment thereof, of claim 7, which comprises the heavy chain amino acid sequence of SEQ ID NO: 21 and the light chain amino acid sequence of SEQ ID NO: 22.

19. The isolated or purified monoclonal antibody, or the antigen-binding fragment thereof, of claim 7, which comprises the heavy chain amino acid sequence of SEQ ID NO: 23 and the light chain amino acid sequence of SEQ ID NO: 24.

20. The isolated or purified monoclonal antibody, or the antigen-binding fragment thereof, of claim 7, which comprises the heavy chain amino acid sequence of SEQ ID NO: 25 and the light chain amino acid sequence of SEQ ID NO: 26.

21. The isolated or purified monoclonal antibody, or the antigen-binding fragment thereof, of claim 7, which comprises the heavy chain amino acid sequence of SEQ ID NO: 27 and the light chain amino acid sequence of SEQ ID NO: 28.

22. The isolated or purified monoclonal antibody, or the antigen-binding fragment thereof, of claim 7, which comprises the heavy chain amino acid sequence of SEQ ID NO: 29 and the light chain amino acid sequence of SEQ ID NO: 30.

23. The isolated or purified monoclonal antibody, or the antigen-binding fragment thereof, of claim 7, which comprises the heavy chain amino acid sequence of SEQ ID NO: 31 and the light chain amino acid sequence of SEQ ID NO: 32.

24. The isolated or purified monoclonal antibody, or the antigen-binding fragment thereof, of claim 7, which comprises the heavy chain amino acid sequence of SEQ ID NO: 33 and the light chain amino acid sequence of SEQ ID NO: 34.

25. The isolated or purified monoclonal antibody, or the antigen-binding fragment thereof, of any one of claims 1 to 6, which is humanized.

26. The isolated or purified monoclonal antibody, or the antigen-binding fragment thereof, of any one of claims 1 to 25, wherein the antigen-binding fragment is a Fab, an F(ab′)₂, and Fab′, or an scFV.

27. The isolated or purified monoclonal antibody, or the antigen-binding fragment thereof, of any one of claims 1 to 26, which has an affinity for human CD3 of 31 nM or less, preferably 30 nM or less, preferably 25 nM or less, preferably 20 nM or less, preferably 15 nM or less, preferably 10 nM or less, preferably 5 nM or less, preferably 2 nM or less, preferably 1 nM or less.

28. The isolated or purified monoclonal antibody, or the antigen-binding fragment thereof, of claim 27, wherein the affinity for human CD3 is as determined by flow cytometry analysis of binding to human T cells.

29. The isolated or purified monoclonal antibody, or the antigen-binding fragment thereof, of any one of claims 1 to 28, which additionally binds to Macaca fascicularis CD3 (GenBank Accession No. GCA_000364345.1).

30. A monoclonal antibody, or an antigen binding fragment thereof, that competes for specific binding to human CD3 with the monoclonal antibody, or the antigen-binding fragment thereof as defined in any one of claims 7 to 24.

31. A recombinant polypeptide comprising one or more monoclonal antibody, or antigen-binding fragment thereof, as defined in any one of claims 1 to 29.

32. A multivalent antibody comprising:
a first antigen-binding portion comprising the monoclonal antibody, or the antigen-binding fragment thereof, as defined in any one of claims 1 to 29, and
a second antigen-binding portion.

33. The multivalent antibody of claim 32, wherein the second antigen-binding portion is an scFv.

34. The multivalent antibody of claim 32 or 33, wherein the second antigen-binding moiety binds specifically to a cell-surface marker.

35. The multivalent antibody of claim 34, wherein the cell-surface marker is a tumour-associated antigen or a tumour-specific antigen.

36. The multivalent antibody of claim 34, wherein the cell-surface marker is human EGFRvII, CD22, BCMA, mesothelin, MUC1, CD19, CD20, CAIX, FAP, or HER2.

37. The multivalent antibody of any one of claim 32 to 36, comprising in N-terminal to C-terminal direction:
the second antigen-binding portion,
an amino acid linker, and
the first antigen-binding portion.

38. The multivalent antibody of claim 37, which further comprises an N-terminal signal peptide.

39. The multivalent antibody of claim 38, wherein the signal peptide is a signal peptide from human CD28, preferably comprising SEQ ID NO: 156.

40. The multivalent antibody of any one of claims 37 to 39, wherein the amino acid linker comprises GGGGS.

41. The multivalent antibody of any one of claims 36 to 40, having an amino acid sequence comprising SEQ ID No: 39.

42. The multivalent antibody of any one of claims 36 to 40, having an amino acid sequence comprising SEQ ID No: 40.

43. The multivalent antibody of any one of claims 36 to 40, having an amino acid sequence comprising SEQ ID No: 43.

44. The multivalent antibody of any one of claims 32 to 43, further comprising a third antigen-binding portion.

45. The multivalent antibody of any one of claims 32 to 44, which comprises an antigen-binding portion that binds to human serum albumin.

46. The multivalent antibody of claim 32 to 45, which is a bispecific T-cell engager.

47. The multivalent antibody of any one of claims 32 to 46, first antigen-binding portion comprises:
a CDRH1 amino acid sequence as set forth in SEQ ID NO: 60,
a CDRH2 amino acid sequence as set forth in SEQ ID NO: 61,
a CDRH3 amino acid sequence as set forth in SEQ ID NO: 62,
a CDRL1 amino acid sequence as set forth in SEQ ID NO: 63,
a CDRL2 amino acid sequence as set forth in SEQ ID NO: 64, and
a CDRL3 amino acid sequence as set forth in SEQ ID NO: 65 (from F322-1E2).

48. The multivalent antibody of claim 47, wherein the first antigen-binding portion comprises SEQ ID NO: 37.

49. The multivalent antibody of claim 47 or 48, wherein the second antigen-biding portion binds to EGFRvIII, preferably wherein the multivalent antibody has the sequence of SEQ ID No: 40.

50. A use of the multivalent antibody as defined in any one of claims 32 to 49 for treatment of a cancer.

51. A method of treating a cancer in subject comprising administering to the subject the multivalent antibody as defined in any one of claims 32 to 49.

52. A chimeric antibody receptor (CAR), which binds to human CD3, comprising the isolated or purified monoclonal antibody, or the antigen-binding fragment thereof, as defined in any one of claims 1 to 29.

53. The CAR of claim 52, which comprises in N-terminal to C-terminal direction:
an CD3 binding domain comprising isolated or purified monoclonal antibody, or the antigen-binding fragment thereof as defined in any one of claims 1 to 29,
a polypeptide hinge,
a transmembrane domain, and
a cytoplasmic domain comprising a signaling domain, preferably wherein the cytoplasmic domain further comprises a co-stimulatory domain.

54. The CAR of claim 53, wherein the polypeptide hinge comprises a human CD8 hinge domain or a fragment thereof, preferably wherein the polypeptide hinge comprises SEQ ID NO: 158.

55. The CAR of claim 53 or 54, wherein the transmembrane domain is a CD28 transmembrane domain, preferably wherein the CD28 transmembrane domain comprises SEQ ID NO: 159.

56. The CAR of any one of claims 53 to 55, wherein the signaling domain is a CD3-zeta signaling domain, preferably wherein the CD3-zeta signaling domain comprises SEQ ID NO: 160.

57. The CAR of any one of claims 53 to 56, wherein the co-stimulatory domain is a 4-1BB co-stimulatory domain, preferably wherein the 4-1 BB signal transduction domain comprises SEQ ID NO: 161.

58. The CAR of any of claims 53 to 57, further comprising a flexible amino acid linker between the CD3 binding domain and the polypeptide hinge, preferably wherein the flexible amino acid linker comprises SEQ ID NO: 162.

59. The CAR of any one of claims 53 to 58, further comprising a signal peptide, preferably wherein the signal peptide is a signal peptide from human CD28, more preferably wherein the signal peptide from human CD28 comprises SEQ ID NO: 163.

60. A recombinant nucleic acid molecule encoding the CAR as defined in any one of claims 53 to 59.

61. A vector comprising the recombinant nucleic acid molecule as defined in claim 60, preferably which is a viral vector, more preferably a lentivirus vector.

62. A recombinant viral particle comprising the recombinant nucleic acid molecule of claim 60, preferably which is a recombinant lentiviral particle.

63. A cell comprising the recombinant nucleic acid molecule of claim 60.

64. An engineered cell expressing at the cell surface membrane the CAR as defined in any one of claims 53 to 59.

65. The engineered cell of claim 64, which is an immune cell, preferably being derived from T-lymphocytes, preferably wherein the immune cell is a T cell.

66. A use of the engineered cell of claim 64 or 65 for treatment of a cancer.

67. A method of treating a cancer in a subject, comprising administering to the subject the engineered cell as defined in claim 64 or 65.

68. A use of the monoclonal antibody, or antigen-binding fragment thereof, as defined in any one of claims 1 to 29 for detection of CD3.

69. A method of detecting of CD3 in sample comprising contacting the sample with the monoclonal antibody, or antigen-binding fragment thereof, as defined any one of claims 1 to 29.

70. A use of the monoclonal antibody, or antigen-binding fragment thereof, as defined any one of claims 1 to 29 for activation of T cells.

71. A method of activating T cells in subject comprising administering to the subject the monoclonal antibody, or antigen-binding fragment thereof, as defined any one of claims 1 to 29.

72. A use of the monoclonal antibody, or antigen-binding fragment thereof, as defined any one of claims 1 to 29 for treatment of a T-cell mediated auto inflammatory disease.

73. A method of treating a T-cell mediated autoinflammatory disease in subject comprising administering to the subject the monoclonal antibody, or antigen-binding fragment thereof, as defined any one of claims 1 to 29.

74. A use of the monoclonal antibody, or antigen-binding fragment thereof, as defined any one of claims 1 to 29 for treatment of a cancer.

75. A method of treating a cancer in subject comprising administering to the subject the monoclonal antibody, or antigen-binding fragment thereof, as defined any one of claims 1 to 29.


SEQUENCE LISTING (165 sequences)

SEQ ID NO: 1 - protein, 119 aa, synthetic construct
  REGION 1..119: note = 1-F282-2D5-2 HC
QVQLQQPGTELVKPGASVKLSCKASGYTFTSYWMHWVKQRPGQGLEWIGNINPSNGGTNYNENFKTKATLTVDKSSSTAYMQLSSLTSEDSAVYYCARDRSGQYYFDYWGQGTTLTVSS

SEQ ID NO: 2 - protein, 112 aa, synthetic construct
  REGION 1..112: note = 2-F282-2D5-2 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCIQSYHLRTFGGGTILEIK

SEQ ID NO: 3 - protein, 119 aa, synthetic construct
  REGION 1..119: note = 3->F332-1C9-2, F332-1A10-2, F332-5H8-2 & F332-8B11-2 HC
QVQLQQPGAELGKPGTSVNLSCKASGYTFTSYWMHWVKQRPGQGLDWIGNINPNSGTTNYNEKFKSKATLTVDKSSSTAYMQLSTLTSEDSAVYYCTRDISGQYYFDYWGQGTSLTVSS

SEQ ID NO: 4 - protein, 112 aa, synthetic construct
  REGION 1..112: note = 4->F332-1C9-2, F332-1A10-2, F332-5H8-2 & F332-8B11-2 LC
DIVMSQSPSSLAVSAGEKVTMSCKSSQSLLNSRTRKNYLAWYQQKPGQSPKLLIYWASTRESGVPDRFTGSGSGTDFTLIISSVQAEDLAVYYCKQSYSLRTFGGGTKLEIK

SEQ ID NO: 5 - protein, 119 aa, synthetic construct
  REGION 1..119: note = 5->F332-1E2-2 & F332-8C5-2 HC
QVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSS

SEQ ID NO: 6 - protein, 112 aa, synthetic construct
  REGION 1..112: note = 6->F332-1E2-2 & F332-8C5-2 LC
DIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIK

SEQ ID NO: 7 - protein, 119 aa, synthetic construct
  REGION 1..119: note = 7->F332-3E9-2 & F332-12G4-2 HC
QVQLQQPGAELGKPGTSVKLSCKASGYTFTSYWIHWLKQRPGQGLEWIGNINPNGGSINCNEKFTTKATLTIDKSSSTAYMQLSTLTSEDFAVYYCTRDTSGQYYFDYWGQGTTLTVSS

SEQ ID NO: 8 - protein, 112 aa, synthetic construct
  REGION 1..112: note = 8->F332-3E9-2 & F332-12G4-2 LC
DIMMSQSPSSLAVSAGEKVTMSCKSSQSLLNSRTRKNYLAWYQQKPGQSPKLLIYWASTRESGVPDRFTGSGSGTDFTLTISSVQAEDLAVYYCTQSYHLRTFGGGTKLEIK

SEQ ID NO: 9 - protein, 119 aa, synthetic construct
  REGION 1..119: note = 9->F332-4D6-2 HC
QVQLQQSGAELGKPGTSVKLSCKASGYTFTSYWIHWVKQRPGQGLEWIGNINPNSGSTNYNEKFKRKATLTADKSSSTAYMQLSTLTSEDFAVYYCTRDTSGQYYFDYWGQGTTLTVSS

SEQ ID NO: 10 - protein, 112 aa, synthetic construct
  REGION 1..112: note = 10->F332-4D6-2 LC
DIVMSQSPSSLAVSAGEKVTMSCKSSQSLLNSRTRKNYLAWYQQKPGQSPKVLIYWASTRESGVPDRFTGSGSGTDFTLTISSVQAEDLAVYYCKQSYHLRTFGGGTKLEIK

SEQ ID NO: 11 - protein, 117 aa, synthetic construct
  REGION 1..117: note = 11->F332-5C7-2 & F332-7C9-2 & F332-7D10-2 HC
QVQLQQSGPELVKPGASVRISCKASGYTFTSHYLHWVKQRPGQGFEWIGWIYPGDGNTRYNEKFKDRATLTADKSSSTAYLHLSSLTSEDSAVYFCARDAGYYFDYWGQGTTLAVSS

SEQ ID NO: 12 - protein, 112 aa, synthetic construct
  REGION 1..112: note = 12->F332-5C7-2 & F332-7C9-2 & F332-7D10-2 LC
DIVMSQSPSSLAVSAGEKVTMSCKSSQSLLNSRTRKNYLAWYQQKPGQSLKVLIYWASTRESGVPDRFTGSGSGTDFTLTISSVQAEDLAVYYCKQSYSLRTFGGGTKLEIK

SEQ ID NO: 13 - protein, 119 aa, synthetic construct
  REGION 1..119: note = 13->F332-5D4-2 & F332-5F8-2 HC
QVQLQQPGAELGKPGTSVKLSCKASGYTFTSYWMHWVKQRPGQGLEWIGNINPNGGDSNYNEKFKSKATLTVDMSSSTAYMQLSTLTSEDSAVYYCTRDISGQYYFDFWGQGTSLTVFS

SEQ ID NO: 14 - protein, 112 aa, synthetic construct
  REGION 1..112: note = 14 >F332-5D4-2 & F332-5F8-2 LC
DILMSQSPSSLAVSAGEQVTMSCKSSQSLLNSRTRKNYLAWYQQKPGQSPKLLIFWASTRKSGVPDRFTGSGSGTYFTLTINSVQTEDLAVYYCTQSYSLRTFGGGTKLEIK

SEQ ID NO: 15 - protein, 119 aa, synthetic construct
  REGION 1..119: note = 15->F332-5E12-2 & F332-7C5-2& F332-10A8-2 HC
QVQLQQPGAELGKPGTSVKLSCKASGYTFTSYWMHWVKQRPGQGLEWIGNINPSSGITNYNERFKNKATLTVDKSSTTAYMQLSILTSEDSAVYYCSRDISGQYYFNFWGQGTTLTVSS

SEQ ID NO: 16 - protein, 112 aa, synthetic construct
  REGION 1..112: note = 16->F332-5E12-2 & F332-7C5-2& F332-10A8-2 LC
DIVMSQSPSSLAVSAGEKVTMSCKSSQSLLNSRTRKNYLAWYQQKPGQSPKLLIYWASTRESGVPDRFTGTGSGTDFTLTISSVQAEDLAVYYCKQSYNLRTFGGGTKLEIK

SEQ ID NO: 17 - protein, 119 aa, synthetic construct
  REGION 1..119: note = 17->F332-5F2-2 & F332-6G8-2 & F332-13A3-2 HC
QVQLQQPGAELGKPGTSVKLSCKASGYTFTSYWMHWVKQRPGQGLEWIGNINPNGGDSNYNEKFKSKATLTVDKSSSTAYMQLSTLTSEDSAVYYCTRDISGQYYFDFWGQGTSLTVFS

SEQ ID NO: 18 - protein, 112 aa, synthetic construct
  REGION 1..112: note = 18->F332-5F2-2 & F332-6G8-2 & F332-13A3-2 LC
DILMSQSPSSLAVSAGEQVTMSCKSSQSLLNSRTRKNYLAWYQQKPGQSPKLLIFWASTRKSGVPDRFTGSGSGTYFTLTISSVQAEDLAVYYCTQSYNLRTFGGGTKLEIK

SEQ ID NO: 19 - protein, 119 aa, synthetic construct; identical to SEQ ID NO: 3
  REGION 1..119: note = 19->F332-6D7-2 & F332-7G4-2 HC

SEQ ID NO: 20 - protein, 112 aa, synthetic construct
  REGION 1..112: note = 20->F332-6D7-2 & F332-7G4-2 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYSLRTFGGGSKLEIK

SEQ ID NO: 21 - protein, 119 aa, synthetic construct
  REGION 1..119: note = 21->F332-7B7-2 HC
QVQLQQPGTELGKPGTSVKLSCKASGYTFTNYWMHWVKQRPGQGLEWIGNINPNSGSTNYNQKFKSKATLTLDKSSSTAYMQFSTLTSEDSAVYYCTRDSSGQYYFDYWGQGTTLTVSS

SEQ ID NO: 22 - protein, 112 aa, synthetic construct
  REGION 1..112: note = 22->F332-7B7-2 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCIQSFHLRTFGGGTKLEIK

SEQ ID NO: 23 - protein, 119 aa, synthetic construct
  REGION 1..119: note = 23->F332-7E3-2 HC
QVQLQQPGAELGKPGTSVKLSCKASGYTFTSYWIHWVKQRPGQGLEWIGNINPNSGSINYNEKFKSKATLTVDKSSSTAYMQLSTLTSEDFAVYYCTRDTSGQYYFDYWGQGTTLTVSS

SEQ ID NO: 24 - protein, 112 aa, synthetic construct
  REGION 1..112: note = 24->F332-7E3-2 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYHLRTFGGGTKLEIK

SEQ ID NO: 25 - protein, 119 aa, synthetic construct
  REGION 1..119: note = 25->F332-10B6-2 & F332-12B5-1 HC
QVQLQQPGAELGKPGTSVNLSCKASGYTFTDYWIHWVKQRPGQGLEWIGNINPNSGDTNYNENFKTKATLTVDRSSSTAYMQLSSLTSEDSAVYYCTRDISGQYYFDFWGQGTTLTVSS

SEQ ID NO: 26 - protein, 112 aa, synthetic construct
  REGION 1..112: note = 26->F332-10B6-2 & F332-12B5-1 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCIQSYNLRTFGGGTKLEIK

SEQ ID NO: 27 - protein, 119 aa, synthetic construct
  REGION 1..119: note = 27->F332-12F6-2 HC
QVQLQQPGAELGKPGTSVKLSCKASGYTFTSYWIHWVKQRPGQGLEWIGNINPNSGSTNYNEKFKSKATLTVDKSSSTAYMQLSTLTSEDSAVYYCTRDISGQYYFDYWGQGTTLTVSS

SEQ ID NO: 28 - protein, 112 aa, synthetic construct
  REGION 1..112: note = 28->F332-12F6-2 LC
DIVMSQSPSSLAVSAGEKVTMSCKSSQSLLNSRTRKSYLAWYQQKPGQSPKLLIYWASTRESGVPDRFTGSGSGTDFTLTISSVQAEDLAVYYCIQSYNLRTFGGGTKLEIK

SEQ ID NO: 29 - protein, 117 aa, synthetic construct
  REGION 1..117: note = 29->F332-13B4-2 HC
QVQLQQSGPELVKPGASVKISCKASGYSFTNSYVHWVKQTPGQGLEWIGWIYPGSTNIKYNEKFKGKATLTADTSSSTVYMQLSSLTSEDSAVYFCARDGSYFFDYWGQGTTLTVSS

SEQ ID NO: 30 - protein, 112 aa, synthetic construct
  REGION 1..112: note = 30->F332-13B4-2 LC
DIVMSQSPSSLAVSAGEKVTMTCKSSQSLLNSRTRKNYLAWYQQKPGQSPKLLIYWASTRESGVPDRFTGSGSGTDFTLTISSVQAEDLAVYYCKQSYILRTFGGGTKLEIK

SEQ ID NO: 31 - protein, 119 aa, synthetic construct
  REGION 1..119: note = 31->F332-8F7-3 HC
QVQLQQPGAELGKPGTSVKLSCKASGYTFTSYWIHWVKQRPGQGLEWIGNINPNSGSTNYNEKFKNKATLTVDKSSSTASMQLSTLTSEDSAVYYCTRDISGQYYFDYWGQGTTLTVSS

SEQ ID NO: 32 - protein, 112 aa, synthetic construct
  REGION 1..112: note = 32->F332-8F7-3 LC
DIVMSQSPSSLAVSTGGEVTMSCKSSQSLLNSRTRKNYLAWYQQKPGQSPKLLIYWASTRESGVPDRFTGSGSGTDFTLTISSVQAEDLAVYYCIQSYSLRTFGGGTKLGIK

SEQ ID NO: 33 - protein, 117 aa, synthetic construct
  REGION 1..117: note = 33->F332-10B9-1, F332-10C9-1 & F332-12A9-1 HC
QVQLQQSGPELVKPGASVRIPCKASGYTFTTYYIHWVKQRPGQGLEWIGWIYPGNGNIKYNEKFKGRATLTADKSSSTAYMQLSSLTSEDSAVYFCARDAGYYFDYWGQGTTLTVSS

SEQ ID NO: 34 - protein, 112 aa, synthetic construct
  REGION 1..112: note = 34->F332-10B9-1, F332-10C9-1 & F332-12A9-1 LC
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYTLRTFGGGTKLEIK

SEQ ID NO: 35 - protein, 243 aa, synthetic construct
  REGION 1..243: note = 35-Single chain variable fragment (scFv) OKT3
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 36 - protein, 534 aa, synthetic construct
  REGION 1..534: note = 36-Modularized CD19-OKT3 sequence
MEFGLSWVFLVALFRGVQCTGGSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSEDFLGGGGSRDDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKVVSAS

SEQ ID NO: 37 - protein, 246 aa, synthetic construct
  REGION 1..246: note = 37-Single chain variable fragment (scFv) derived from F332-1E2
QVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIK

SEQ ID NO: 38 - protein, 278 aa, synthetic construct
  REGION 1..278: note = 38-EGFRvIII-specific single chain variable fragment (scFv)derived from F269-3D12
EVQLQQSGPELVKPGSSVRISCKASGYTFTDYNMDWVKQSHGKSLEWIGTINPNNGGTSYNQKFKGKATLTVDKSSSTAYMELRSLTSEDSAVYYCARVRQLGLWFAYWGQGTLVTVSAAKTTPPSVYPLAPGSLGGTGGGSGGGGSGGGGSEIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGSGTKLEIKRADAAPTVSIFPPSSKLG

SEQ ID NO: 39 - protein, 558 aa, synthetic construct
  REGION 1..558: note = 39-EGFRvIII-CD3 (F269-3D12-OKT3) Bispecific Antibody Sequence
MEFGLSWVFLVALFRGVQCTGEVQLQQSGPELVKPGSSVRISCKASGYTFTDYNMDWVKQSHGKSLEWIGTINPNNGGTSYNQKFKGKATLTVDKSSSTAYMELRSLTSEDSAVYYCARVRQLGLWFAYWGQGTLVTVSAAKTTPPSVYPLAPGSLGGTGGGSGGGGSGGGGSEIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGSGTKLEIKRADAAPTVSIFPPSSKLGDLGGGGSRDDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 40 - protein, 558 aa, synthetic construct
  REGION 1..558: note = 40-EGFRvIII-CD3 (F269-3D12-F322-1E2) Bispecific Antibody Sequence
MEFGLSWVFLVALFRGVQCTGEVQLQQSGPELVKPGSSVRISCKASGYTFTDYNMDWVKQSHGKSLEWIGTINPNNGGTSYNQKFKGKATLTVDKSSSTAYMELRSLTSEDSAVYYCARVRQLGLWFAYWGQGTLVTVSAAKTTPPSVYPLAPGSLGGTGGGSGGGGSGGGGSEIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGSGTKLEIKRADAAPTVSIFPPSSKLGDLGGGGSQVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKHHHHHH

SEQ ID NO: 41 - protein, 116 aa, synthetic construct
  REGION 1..116: note = 41-EGFR-specific camelid single domain antibody sequence(sdAb021)
QVQVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSS

SEQ ID NO: 42 - protein, 45 aa, synthetic construct
  REGION 1..45: note = 42-Human CD8-alpha hinge domain
TTTPAPRPPTPAPTIASQPLSLRPEACRPAAGGAVHTRGLDFACD

SEQ ID NO: 43 - protein, 463 aa, synthetic construct
  REGION 1..463: note = 43-EGFRsdAb-CD3 (sdAb021-F322-1E2) Bispecific Antibody Sequence
MEFGLSWVFLVALFRGVQCTGQVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSSGRLPLRDDGGGGSGGGGSGGGGSGGTTTPAPRPPTPAPTIASQPLSLRPEACRPAAGGAVHTRGLDFACDLGGGGSQVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKHHHHHH

SEQ ID NO: 44 - protein, 219 aa, synthetic construct
  REGION 1..219: note = 44-F322-1E2-Fab Light chain
DIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 241 aa, synthetic construct
  REGION 1..241: note = 45-F322-1E2-Fab Heavy Chain
QVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGGAPALQPTQGAMPA

SEQ ID NO: 46 - protein, 219 aa, synthetic construct
  REGION 1..219: note = 46-F322-4D6-Fab Light chain
DIVMSQSPSSLAVSAGEKVTMSCKSSQSLLNSRTRKNYLAWYQQKPGQSPKVLIYWASTRESGVPDRFTGSGSGTDFTLTISSVQAEDLAVYYCKQSYHLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 241 aa, synthetic construct
  REGION 1..241: note = 47-F322-4D6-Fab Heavy Chain
QVQLQQSGAELGKPGTSVKLSCKASGYTFTSYWIHWVKQRPGQGLEWIGNINPNSGSTNYNEKFKRKATLTADKSSSTAYMQLSTLTSEDFAVYYCTRDTSGQYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGGAPALQPTQGAMPA

SEQ ID NO: 48 - protein, 5 aa, synthetic construct
  REGION 1..5: note = 48-F282-2D5-2 CDRH1
SYWMH

SEQ ID NO: 49 - protein, 17 aa, synthetic construct
  REGION 1..17: note = 49-F282-2D5-2 CDRH2
NINPSNGGTNYNENFKT

SEQ ID NO: 50 - protein, 10 aa, synthetic construct
  REGION 1..10: note = 50-F282-2D5-2 CDRH3
DRSGQYYFDY

SEQ ID NO: 51 - protein, 17 aa, synthetic construct
  REGION 1..17: note = 51-F282-2D5-2 CDRL1
KSSQSLLNSRTRKNYLA

SEQ ID NO: 52 - protein, 7 aa, synthetic construct
  REGION 1..7: note = 52-F282-2D5-2 CDRL2
WASTRES

SEQ ID NO: 53 - protein, 8 aa, synthetic construct
  REGION 1..8: note = 53-F282-2D5-2 CDRL3
IQSYHLRT

SEQ ID NO: 54 - protein, 5 aa, synthetic construct
  REGION 1..5: note = 54->F332-1C9-2, F332-1A10-2, F332-5H8-2 & F332-8B11-2 CDRH1
SYWMH

SEQ ID NO: 55 - protein, 17 aa, synthetic construct
  REGION 1..17: note = 55->F332-1C9-2, F332-1A10-2, F332-5H8-2 & F332-8B11-2 CDRH2
NINPNSGTTNYNEKFKS

SEQ ID NO: 56 - protein, 10 aa, synthetic construct
  REGION 1..10: note = 56 >F332-1C9-2, F332-1A10-2, F332-5H8-2 & F332-8B11-2 CDRH3
DISGQYYFDY

SEQ ID NO: 57 - protein, 17 aa, synthetic construct
  REGION 1..17: note = 57->F332-1C9-2, F332-1A10-2, F332-5H8-2 & F332-8B11-2 CDRL1
KSSQSLLNSRTRKNYLA

SEQ ID NO: 58 - protein, 7 aa, synthetic construct
  REGION 1..7: note = 58->F332-1C9-2, F332-1A10-2, F332-5H8-2 & F332-8B11-2 CDRL2
WASTRES

SEQ ID NO: 59 - protein, 8 aa, synthetic construct
  REGION 1..8: note = 59->F332-1C9-2, F332-1A10-2, F332-5H8-2 & F332-8B11-2 CDRL3
KQSYSLRT

SEQ ID NO: 60 - protein, 5 aa, synthetic construct
  REGION 1..5: note = 60->F332-1E2-2 & F332-8C5-2 CDRH1
SYWIH

SEQ ID NO: 61 - protein, 17 aa, synthetic construct
  REGION 1..17: note = 61->F332-1E2-2 & F332-8C5-2 CDRH2
NINPNSGSINYNEKFKN

SEQ ID NO: 62 - protein, 10 aa, synthetic construct
  REGION 1..10: note = 62->F332-1E2-2 & F332-8C5-2 CDRH3
DTSGQYYFDY

SEQ ID NO: 63 - protein, 17 aa, synthetic construct
  REGION 1..17: note = 63->F332-1E2-2 & F332-8C5-2 CDRL1
KSSQSLLNSRTRKNYLA

SEQ ID NO: 64 - protein, 7 aa, synthetic construct
  REGION 1..7: note = 64->F332-1E2-2 & F332-8C5-2 CDRL2
WASTRES

SEQ ID NO: 65 - protein, 8 aa, synthetic construct
  REGION 1..8: note = 65->F332-1E2-2 & F332-8C5-2 CDRL3
IQSYHLRT

SEQ ID NO: 66 - protein, 5 aa, synthetic construct
  REGION 1..5: note = 66->F332-3E9-2 & F332-12G4-2 CDRH1
SYWIH

SEQ ID NO: 67 - protein, 17 aa, synthetic construct
  REGION 1..17: note = 67->F332-3E9-2 & F332-12G4-2 CDRH2
NINPNGGSINCNEKFTT

SEQ ID NO: 68 - protein, 10 aa, synthetic construct
  REGION 1..10: note = 68->F332-3E9-2 & F332-12G4-2 CDRH3
DTSGQYYFDY

SEQ ID NO: 69 - protein, 17 aa, synthetic construct
  REGION 1..17: note = 69->F332-3E9-2 & F332-12G4-2 CDRL1
KSSQSLLNSRTRKNYLA

SEQ ID NO: 70 - protein, 7 aa, synthetic construct
  REGION 1..7: note = 70->F332-3E9-2 & F332-12G4-2 CDRL2
WASTRES

SEQ ID NO: 71 - protein, 8 aa, synthetic construct
  REGION 1..8: note = 71->F332-3E9-2 & F332-12G4-2 CDRL3
TQSYHLRT

SEQ ID NO: 72 - protein, 5 aa, synthetic construct
  REGION 1..5: note = 72->F332-4D6-2 CDRH1
SYWIH

SEQ ID NO: 73 - protein, 17 aa, synthetic construct
  REGION 1..17: note = 73->F332-4D6-2 CDRH2
NINPNSGSTNYNEKFKR

SEQ ID NO: 74 - protein, 10 aa, synthetic construct
  REGION 1..10: note = 74->F332-4D6-2 CDRH3
DTSGQYYFDY

SEQ ID NO: 75 - protein, 17 aa, synthetic construct
  REGION 1..17: note = 75->F332-4D6-2 CDRL1
KSSQSLLNSRTRKNYLA

SEQ ID NO: 76 - protein, 7 aa, synthetic construct
  REGION 1..7: note = 76->F332-4D6-2 CDRL2
WASTRES

SEQ ID NO: 77 - protein, 8 aa, synthetic construct
  REGION 1..8: note = 77->F332-4D6-2 CDRL3
KQSYHLRT

SEQ ID NO: 78 - protein, 5 aa, synthetic construct
  REGION 1..5: note = 78->F332-5C7-2 & F332-7C9-2 & F332-7D10-2 CDRH1
SHYLH

SEQ ID NO: 79 - protein, 17 aa, synthetic construct
  REGION 1..17: note = 79->F332-5C7-2 & F332-7C9-2 & F332-7D10-2 CDRH2
WIYPGDGNTRYNEKFKD

SEQ ID NO: 80 - protein, 8 aa, synthetic construct
  REGION 1..8: note = 80->F332-5C7-2 & F332-7C9-2 & F332-7D10-2 CDRH3
DAGYYFDY

SEQ ID NO: 81 - protein, 17 aa, synthetic construct
  REGION 1..17: note = 81->F332-5C7-2 & F332-7C9-2 & F332-7D10-2 CDRL1
KSSQSLLNSRTRKNYLA

SEQ ID NO: 82 - protein, 7 aa, synthetic construct
  REGION 1..7: note = 82->F332-5C7-2 & F332-7C9-2 & F332-7D10-2 CDRL2
WASTRES

SEQ ID NO: 83 - protein, 8 aa, synthetic construct
  REGION 1..8: note = 83->F332-5C7-2 & F332-7C9-2 & F332-7D10-2 CDRL3
KQSYSLRT

SEQ ID NO: 84 - protein, 5 aa, synthetic construct
  REGION 1..5: note = 84->F332-5D4-2 & F332-5F8-2 CDRH1
SYWMH

SEQ ID NO: 85 - protein, 17 aa, synthetic construct
  REGION 1..17: note = 85->F332-5D4-2 & F332-5F8-2 CDRH2
NINPNGGDSNYNEKFKS

SEQ ID NO: 86 - protein, 10 aa, synthetic construct
  REGION 1..10: note = 86->F332-5D4-2 & F332-5F8-2 CDRH3
DISGQYYFDF

SEQ ID NO: 87 - protein, 17 aa, synthetic construct
  REGION 1..17: note = 87->F332-5D4-2 & F332-5F8-2 CDRL1
KSSQSLLNSRTRKNYLA

SEQ ID NO: 88 - protein, 7 aa, synthetic construct
  REGION 1..7: note = 88->F332-5D4-2 & F332-5F8-2 CDRL2
WASTRKS

SEQ ID NO: 89 - protein, 8 aa, synthetic construct
  REGION 1..8: note = 89->F332-5D4-2 & F332-5F8-2 CDRL3
TQSYSLRT

SEQ ID NO: 90 - protein, 5 aa, synthetic construct
  REGION 1..5: note = 90->F332-5E12-2 & F332-7C5-2& F332-10A8-2 CDRH1
SYWMH

SEQ ID NO: 91 - protein, 17 aa, synthetic construct
  REGION 1..17: note = 91->F332-5E12-2 & F332-7C5-2& F332-10A8-2 CDRH2
NINPSSGITNYNERFKN

SEQ ID NO: 92 - protein, 10 aa, synthetic construct
  REGION 1..10: note = 92->F332-5E12-2 & F332-7C5-2& F332-10A8-2 CDRH3
DISGQYYFNF

SEQ ID NO: 93 - protein, 17 aa, synthetic construct
  REGION 1..17: note = 93->F332-5E12-2 & F332-7C5-2& F332-10A8-2 CDRL1
KSSQSLLNSRTRKNYLA

SEQ ID NO: 94 - protein, 7 aa, synthetic construct
  REGION 1..7: note = 94->F332-5E12-2 & F332-7C5-2& F332-10A8-2 CDRL2
WASTRES

SEQ ID NO: 95 - protein, 8 aa, synthetic construct
  REGION 1..8: note = 95->F332-5E12-2 & F332-7C5-2& F332-10A8-2 CDRL3
KQSYNLRT

SEQ ID NO: 96 - protein, 5 aa, synthetic construct
  REGION 1..5: note = 96->F332-5F2-2 & F332-6G8-2 & F332-13A3-2 CDRH1
SYWMH

SEQ ID NO: 97 - protein, 17 aa, synthetic construct
  REGION 1..17: note = 97->F332-5F2-2 & F332-6G8-2 & F332-13A3-2 CDRH2
NINPNGGDSNYNEKFKS

SEQ ID NO: 98 - protein, 10 aa, synthetic construct
  REGION 1..10: note = 98->F332-5F2-2 & F332-6G8-2 & F332-13A3-2 CDRH3
DISGQYYFDF

SEQ ID NO: 99 - protein, 17 aa, synthetic construct
  REGION 1..17: note = 99->F332-5F2-2 & F332-6G8-2 & F332-13A3-2 CDRL1
KSSQSLLNSRTRKNYLA

SEQ ID NO: 100 - protein, 7 aa, synthetic construct
  REGION 1..7: note = 100->F332-5F2-2 & F332-6G8-2 & F332-13A3-2 CDRL2
WASTRKS

SEQ ID NO: 101 - protein, 8 aa, synthetic construct
  REGION 1..8: note = 101->F332-5F2-2 & F332-6G8-2 & F332-13A3-2 CDRL3
TQSYNLRT

SEQ ID NO: 102 - protein, 5 aa, synthetic construct
  REGION 1..5: note = 102->F332-6D7-2 & F332-7G4-2 CDRH1
SYWMH

SEQ ID NO: 103 - protein, 16 aa, synthetic construct
  REGION 1..16: note = 103->F332-6D7-2 & F332-7G4-2 CDRH2
NINPNSGTTNYNEKFK

SEQ ID NO: 104 - protein, 10 aa, synthetic construct
  REGION 1..10: note = 104->F332-6D7-2 & F332-7G4-2 CDRH3
DISGQYYFDY

SEQ ID NO: 105 - protein, 17 aa, synthetic construct
  REGION 1..17: note = 105->F332-6D7-2 & F332-7G4-2 CDRL1
KSSQSLLNSRTRKNYLA

SEQ ID NO: 106 - protein, 7 aa, synthetic construct
  REGION 1..7: note = 106->F332-6D7-2 & F332-7G4-2 CDRL2
WASTRES

SEQ ID NO: 107 - protein, 8 aa, synthetic construct
  REGION 1..8: note = 107->F332-6D7-2 & F332-7G4-2 CDRL3
KQSYSLRT

SEQ ID NO: 108 - protein, 5 aa, synthetic construct
  REGION 1..5: note = 108->F332-7B7-2 CDRH1
NYWMH

SEQ ID NO: 109 - protein, 17 aa, synthetic construct
  REGION 1..17: note = 109->F332-7B7-2 CDRH2
NINPNSGSTNYNQKFKS

SEQ ID NO: 110 - protein, 10 aa, synthetic construct
  REGION 1..10: note = 110->F332-7B7-2 CDRH3
DSSGQYYFDY

SEQ ID NO: 111 - protein, 17 aa, synthetic construct
  REGION 1..17: note = 111->F332-7B7-2 CDRL1
KSSQSLLNSRTRKNYLA

SEQ ID NO: 112 - protein, 7 aa, synthetic construct
  REGION 1..7: note = 112->F332-7B7-2 CDRL2
WASTRES

SEQ ID NO: 113 - protein, 8 aa, synthetic construct
  REGION 1..8: note = 113->F332-7B7-2 CDRL3
IQSFHLRT

SEQ ID NO: 114 - protein, 5 aa, synthetic construct
  REGION 1..5: note = 114->F332-7E3-2 CDRH1
SYWIH

SEQ ID NO: 115 - protein, 17 aa, synthetic construct
  REGION 1..17: note = 115->F332-7E3-2 CDRH2
NINPNSGSINYNEKFKS

SEQ ID NO: 116 - protein, 10 aa, synthetic construct
  REGION 1..10: note = 116->F332-7E3-2 CDRH3
DTSGQYYFDY

SEQ ID NO: 117 - protein, 17 aa, synthetic construct
  REGION 1..17: note = 117->F332-7E3-2 CDRL1
KSSQSLLNSRTRKNYLA

SEQ ID NO: 118 - protein, 7 aa, synthetic construct
  REGION 1..7: note = 118->F332-7E3-2 CDRL2
WASTRES

SEQ ID NO: 119 - protein, 8 aa, synthetic construct
  REGION 1..8: note = 119->F332-7E3-2 CDRL3
KQSYHLRT

SEQ ID NO: 120 - protein, 5 aa, synthetic construct
  REGION 1..5: note = 120->F332-10B6-2 & F332-12B5-1 CDRH1
DYWIH

SEQ ID NO: 121 - protein, 17 aa, synthetic construct
  REGION 1..17: note = 121->F332-10B6-2 & F332-12B5-1 CDRH2
NINPNSGDTNYNENFKT

SEQ ID NO: 122 - protein, 10 aa, synthetic construct
  REGION 1..10: note = 122->F332-10B6-2 & F332-12B5-1 CDRH3
DISGQYYFDF

SEQ ID NO: 123 - protein, 17 aa, synthetic construct
  REGION 1..17: note = 123->F332-10B6-2 & F332-12B5-1 CDRL1
KSSQSLLNSRTRKNYLA

SEQ ID NO: 124 - protein, 7 aa, synthetic construct
  REGION 1..7: note = 124->F332-10B6-2 & F332-12B5-1 CDRL2
WASTRES

SEQ ID NO: 125 - protein, 8 aa, synthetic construct
  REGION 1..8: note = 125->F332-10B6-2 & F332-12B5-1 CDRL3
IQSYNLRT

SEQ ID NO: 126 - protein, 5 aa, synthetic construct
  REGION 1..5: note = 126->F332-12F6-2 CDRH1
SYWIH

SEQ ID NO: 127 - protein, 17 aa, synthetic construct
  REGION 1..17: note = 127->F332-12F6-2 CDRH2
NINPNSGSTNYNEKFKS

SEQ ID NO: 128 - protein, 10 aa, synthetic construct
  REGION 1..10: note = 128->F332-12F6-2 CDRH3
DISGQYYFDY

SEQ ID NO: 129 - protein, 17 aa, synthetic construct
  REGION 1..17: note = 129->F332-12F6-2 CDRL1
KSSQSLLNSRTRKSYLA

SEQ ID NO: 130 - protein, 7 aa, synthetic construct
  REGION 1..7: note = 130->F332-12F6-2 CDRL2
WASTRES

SEQ ID NO: 131 - protein, 8 aa, synthetic construct
  REGION 1..8: note = 131->F332-12F6-2 CDRL3
IQSYNLRT

SEQ ID NO: 132 - protein, 5 aa, synthetic construct
  REGION 1..5: note = 132->F332-13B4-2 CDRH1
NSYVH

SEQ ID NO: 133 - protein, 17 aa, synthetic construct
  REGION 1..17: note = 133->F332-13B4-2 CDRH2
WIYPGSTNIKYNEKFKG

SEQ ID NO: 134 - protein, 8 aa, synthetic construct
  REGION 1..8: note = 134->F332-13B4-2 CDRH3
DGSYFFDY

SEQ ID NO: 135 - protein, 17 aa, synthetic construct
  REGION 1..17: note = 135->F332-13B4-2 CDRL1
KSSQSLLNSRTRKNYLA

SEQ ID NO: 136 - protein, 7 aa, synthetic construct
  REGION 1..7: note = 136->F332-13B4-2 CDRL2
WASTRES

SEQ ID NO: 137 - protein, 8 aa, synthetic construct
  REGION 1..8: note = 137->F332-13B4-2 CDRL3
KQSYILRT

SEQ ID NO: 138 - protein, 5 aa, synthetic construct
  REGION 1..5: note = 138->F332-8F7-3 CDRH1
SYWIH

SEQ ID NO: 139 - protein, 17 aa, synthetic construct
  REGION 1..17: note = 139->F332-8F7-3 CDRH2
NINPNSGSTNYNEKFKN

SEQ ID NO: 140 - protein, 10 aa, synthetic construct
  REGION 1..10: note = 140->F332-8F7-3 CDRH3
DISGQYYFDY

SEQ ID NO: 141 - protein, 17 aa, synthetic construct
  REGION 1..17: note = 141->F332-8F7-3 CDRL1
KSSQSLLNSRTRKNYLA

SEQ ID NO: 142 - protein, 7 aa, synthetic construct
  REGION 1..7: note = 142->F332-8F7-3 CDRL2
WASTRES

SEQ ID NO: 143 - protein, 8 aa, synthetic construct
  REGION 1..8: note = 143->F332-8F7-3 CDRL3
IQSYSLRT

SEQ ID NO: 144 - protein, 5 aa, synthetic construct
  REGION 1..5: note = 144->F332-10B9-1, F332-10C9-1 & F332-12A9-1 CDRH1
TYYIH

SEQ ID NO: 145 - protein, 17 aa, synthetic construct
  REGION 1..17: note = 145->F332-10B9-1, F332-10C9-1 & F332-12A9-1 CDRH2
WIYPGNGNIKYNEKFKG

SEQ ID NO: 146 - protein, 8 aa, synthetic construct
  REGION 1..8: note = 146->F332-10B9-1, F332-10C9-1 & F332-12A9-1 CDRH3
DAGYYFDY

SEQ ID NO: 147 - protein, 17 aa, synthetic construct
  REGION 1..17: note = 147->F332-10B9-1, F332-10C9-1 & F332-12A9-1 CDRL1
KSSQSLLNSRTRKNYLA

SEQ ID NO: 148 - protein, 7 aa, synthetic construct
  REGION 1..7: note = 148->F332-10B9-1, F332-10C9-1 & F332-12A9-1 CDRL2
WASTRES

SEQ ID NO: 149 - protein, 8 aa, synthetic construct
  REGION 1..8: note = 149->F332-10B9-1, F332-10C9-1 & F332-12A9-1 CDRL3
KQSYTLRT

SEQ ID NO: 150 - skipped

SEQ ID NO: 151 - protein, 17 aa, synthetic construct
  REGION 1..17: note = 151-Consensus CDRH2
  SITE 1: note = MISC_FEATURE - Xaa is N or W
  SITE 3: note = MISC_FEATURE - Xaa is N or Y
  SITE 5: note = MISC_FEATURE - Xaa is S, N, or G
  SITE 6: note = MISC_FEATURE - Xaa is N, S, G, or D
  SITE 7: note = MISC_FEATURE - Xaa is G or T
  SITE 8: note = MISC_FEATURE - Xaa is G, T, S, N, D, or I
  SITE 9: note = MISC_FEATURE - Xaa is T, I, or S
  SITE 10: note = MISC_FEATURE - Xaa is N, R, or K
  SITE 11: note = MISC_FEATURE - Xaa is Y or C
  SITE 13: note = MISC_FEATURE - Xaa is E or Q
  SITE 14: note = MISC_FEATURE - Xaa is N, K, or R
  SITE 16: note = MISC_FEATURE - Xaa is K or T
  SITE 17: note = MISC_FEATURE - Xaa is T, S, N, R, D, or G
XIXPXXXXXXXNXXFXX

SEQ ID NO: 152 - skipped

SEQ ID NO: 153 - protein, 17 aa, synthetic construct
  REGION 1..17: note = 153-Consensus CDRL1
  SITE 14: note = MISC_FEATURE - Xaa is N or S
KSSQSLLNSRTRKXYLA

SEQ ID NO: 154 - protein, 7 aa, synthetic construct
  REGION 1..7: note = 154-Consensus CDRL2
  SITE 6: note = MISC_FEATURE - Xaa is E or K
WASTRXS

SEQ ID NO: 155 - protein, 8 aa, synthetic construct
  REGION 1..8: note = 155-Consensus CDRL3
  SITE 1: note = MISC_FEATURE - Xaa is K, I, or T
  SITE 4: note = MISC_FEATURE - Xaa is Y or F
  SITE 5: note = MISC_FEATURE - Xaa is H, S, I, N, or T
XQSXXLRT

SEQ ID NO: 156 - protein, 21 aa, synthetic construct
  REGION 1..21: note = 156-Signal Peptide
MEFGLSWVFLVALFRGVQCTG

SEQ ID NO: 157 - protein, 18 aa, synthetic construct
  REGION 1..18: note = 157-Artificial Glycosylation Sequon
GGGGGAPALQPTQGAMPA

SEQ ID NO: 158 - protein, 45 aa, synthetic construct; identical to SEQ ID NO: 42
  REGION 1..45: note = 158-Human CD8 Hinge Domain

SEQ ID NO: 159 - protein, 32 aa, synthetic construct
  REGION 1..32: note = 159-Human CD28 Transmembrane Domain
PSKPFWVLVVVGGVLACYSLLVTVAFIIFWVR

SEQ ID NO: 160 - protein, 114 aa, synthetic construct
  REGION 1..114: note = 160-Human CD3zeta Signaling Domain
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 161 - protein, 42 aa, synthetic construct
  REGION 1..42: note = 161-Human 4-1BB Co-stimulatory Domain
KRGRKKLLYIFKQPFMRPVQTTQEEDGCSCRFPEEEEGGCEL

SEQ ID NO: 162 - protein, 17 aa, synthetic construct
  REGION 1..17: note = 162-Flexible linker
GGGGSGGGGSGGGGSGG

SEQ ID NO: 163 - protein, 18 aa, synthetic construct
  REGION 1..18: note = 163-Human CD28 Signal Peptide
MLRLLLALNLFPSIQVTG

SEQ ID NO: 164 - protein, 5 aa, synthetic construct
  REGION 1..5: note = 164-Linker
GGGGS

SEQ ID NO: 165 - protein, 15 aa, synthetic construct
  REGION 1..15: note = 165-Linker
GGGGSGGGGSGGGGS